(19) United States
(12) Patent Application Publication
(10) Pub. No.: US 2006/0235035 A1
(43) Pub. Date: Oct. 19, 2006

(54) Novel methoxybenzamibe compounds for use in mch receptor related disorders

(75) Inventors: Thomas Hogberg, Akarp (SE); Emelie Bjurling, Malmo (SE); Jean-Marie Receveur, Frederiksberg (FR); Trond Ulven, Copenhagen K (DK); Paul B. Little, Copenhagen K (DK); Christian E. Elling, Horsholm (DK); Pia K. Norregaard, Lyngby (DK)

Correspondence Address: EDWARDS & ANGELL, LLP, P.O. BOX 55874, BOSTON, MA, 02205, US

(73) Assignee: 7TM Pharma A/S, Horsholm (DK)

(21) Appl. No.: 10/510,907

(22) PCT Filed: Apr. 8, 2003
(86) PCT No.: PCT/DK03/00231

§ 371 Date: May 16, 2005

(30) Foreign Application Priority Data
Apr. 9, 2002 (DK) PA 2002 00519
Apr. 9, 2002 (DK) PA 2002 00520
Apr. 9, 2002 (DK) PA 2002 00524
Nov. 25, 2002 (DK) PA 2002 01818

(51) Int. Cl. A61K 31/513; A61K 31/4747; A61K 31/4745; A61K 31/4035; A61K 31/4709; A61K 31/381; A61K 31/35; A61K 31/34

(52) U.S. Cl. 514/269; 514/278; 514/306; 514/471; 514/451; 514/621; 514/415; 514/447; 544/314; 546/15; 546/138; 549/76; 546/148; 549/480; 549/484

(57) ABSTRACT

Novel compounds of Formula (I) which modulate MCH activity are disclosed, in which A is a linker; Ar₁ is an aryl or heteroaryl group; R1 is a lower alkoxy group; R2 is an R1 group or hydrogen, an OH or an NH₂ group, Q together with the carbonyl forms an amide group, which is further substituted with an amine group; R5 is selected from hydrogen, halogen atoms, alkoxy groups, hydroxy, alkylamino groups, dialkylamino groups, hydroxylalkyl groups, carboxamido groups, acylamido groups, acyl groups, —CHO, nitrile, alkyl, alkenyl or alkynyl groups, —SCH₃, partially or fully fluorinated alkyl, alkoxy or thioalkoxy groups such as —CH₂CF₃, —CF₂CF₃, —CF₃, —OCF₃, —SCF₃; —SO₂NH₂, —SO₂NHAlk, —SO₂NAlk₂, —SO₂Alk; X is H, F, Cl, Br, I, —SCH₃, —CF₃, —OCF₃, —SCF₃, OCH₃, or lower alkyl or alkenyl group; R8 is halogen atoms, alkyl, alkenyl or alkynyl groups, cycloalkyl groups, aryl groups, heteroaryl groups, heterocyclyl groups, alkylcycloalkyl groups, alkylaryl groups, alkylheterocyclyl groups, alkylheteroaryl groups, arylalkoxy groups, aryloxy groups, alkoxy groups, dialkylamino groups, —CONHAlk, —CONHAr, —CONAlk₂, —NHCO-Alk, —NHCO—Ar, —CO-Alk, —CO—Ar, —SCH₃, partially or fully fluorinated alkyl, alkoxy or thioalkoxy groups; or R8 is R6-Ar₂—B—, in which B is a single bond or a connecting moiety; Ar₂ is an Ar₁ group; R6 is an R5 group; and which are useful in the treatment or prevention of e.g. obesity, depression, diabetes, bulimia etc.

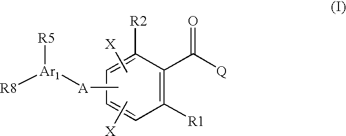

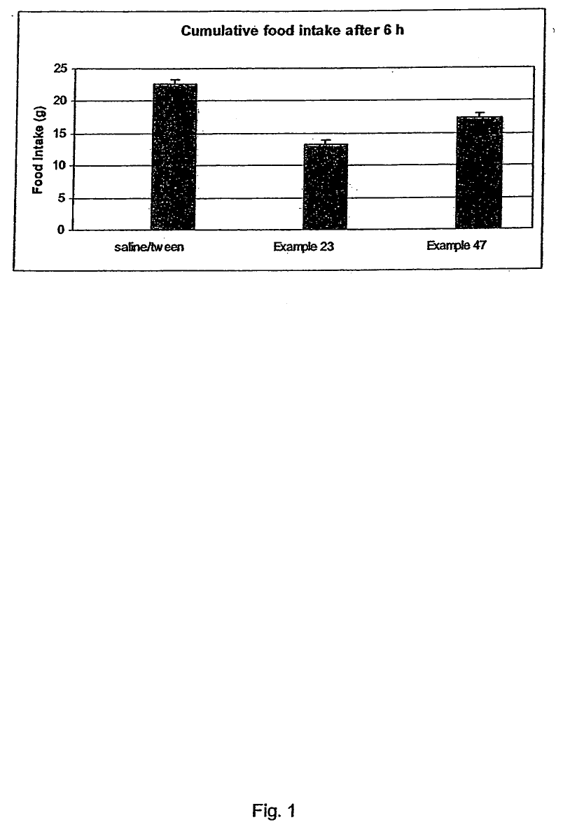

### FIELD OF THE INVENTION

The present invention relates to novel compounds that interact with a melanin-concentrating hormone receptor, a MCH receptor. The compounds have modulating activity on the MCH receptor such as e.g. antagonistic, agonistic or allosteric activity and are useful for medicinal or cosmetic purposes such as, e.g. in the treatment or prevention of feeding disorders like obesity, metabolic syndrome, Type II diabetes, bulimia etc. or in the treatment or prevention of depression.

The invention also relates to therapeutic and/or prophylactic use of the compounds, to processes for the preparation of the novel compounds, to pharmaceutical compositions comprising the compounds, to the manufacture of such compositions and to methods for the treatment and/or prevention of MCH receptor related disorders.

### BACKGROUND OF THE INVENTION

Melanin-concentrating hormone (MCH) is a cyclic peptide that originally was isolated from salmoid pituitaries. In the fish, the 17 amino acid peptide causes aggregation of melanin and inhibits the release of ACTH. Mammalian MCH (19 amino acids) is highly conserved between rat, mouse and human exhibiting 100% amino acid identity. In the last decades there has been increasing activity in the research in the physiologic roles of MCH. It has been reported that MCH is involved in the feeding or body weight regulation, in energy balance, in response to stress, in water balance, in energy metabolism, in the general arousal/attention state, memory and cognitive functions and in psychiatric disorders. The biological effects of MCH are believed to be mediated by specific MCH receptors, and the MCH1 and MCH2 receptors have been described. Antagonists of MCH receptor (e.g. MCH1 receptor) may be suitable for use as obesity or weight reducing agents and they are also believed to have antidepressant and/or anxiolytic properties.

The present invention provides novel compounds that have a MCH modulating activity, i.e. antagonistic, inverse agonistic/negative antagonism, allosteric modulator, partial agonist or agonistic action.

### DETAILED DESCRIPTION OF THE INVENTION

In the present context the following definitions apply:

In the present context, the term “alkyl” is intended to indicate a branched or straight-chain, saturated chemical group containing 1-8 carbon atoms such as, e.g. 1-6 carbon atoms, such as methyl, ethyl, propyl, isopropyl, butyl, sec. butyl, tert. butyl, pentyl, isopentyl, hexyl, isohexyl, heptyl, octyl etc.

The term “lower alkyl” is intended to indicate an alkyl group containing 1-6 carbon atoms, such as, .e.g, 1-4 carbon atoms, unless otherwise specified. Likewise, “lower alkenyl” and “lower alkynyl” are intended to indicate alkenyl and alkynyl groups, respectively containing 2-6 carbon atoms.

The term “alkenyl” is intended to indicate an unsaturated alkyl group having one or more double bonds and 2-8 carbon atoms unless otherwise specified.

The term “alkynyl” is intended to indicate an unsaturated alkyl group having one or more triple bonds and 2-8 carbon atoms unless otherwise specified.

The term “cycloalkyl” is intended to denote a cyclic, saturated alkyl group of 3-7 carbon atoms.

The term “cycloalkenyl” is intended to denote a cyclic, unsaturated alkyl group of 5-7 carbon atoms having one or more double bonds.

The term “alkoxy” is intended to indicate the group alkyl-O—.

The term “aryl” is intended to denote an aromatic (unsaturated), typically 6-membered, ring, which may be a single ring (e.g. phenyl) or fused with other 5- or 6-membered rings (e.g. naphthyl or indole).

The term “heteroaryl” is intended to denote an aromatic (unsaturated), 5- or 6-membered, ring, which may be a single ring (e.g. pyridyl) or fused with other 5- or 6-membered rings (e.g. quinoline or indole).

The term “heterocyclyl” is intended to indicate a cyclic unsaturated (heteroalkenyl), aromatic (“heteroaryl”) or saturated (“heterocycloalkyl”) group comprising at least one heteroatom.

The present invention relates to a compound with the following structure (Formula I) 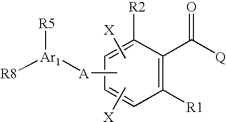
wherein -A- is a linker, which is selected from the group consisting of 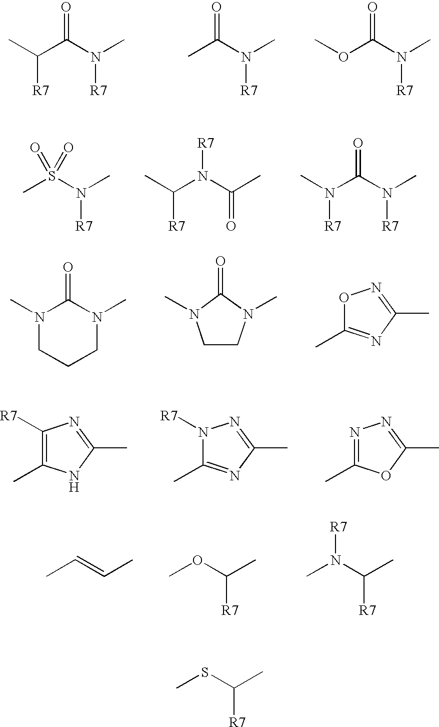
and, wherein the linker -A- may be attached via either of the two free bonds to the Ar₁ group;
and R7 is the same or different and is hydrogen or a straight or branched C₁-C₄ alkyl or alkenyl group;
Ar₁ is an aryl or heteroaryl group such as, e.g. phenyl, pyridine, pyrimidine, pyrazine, thiophene, oxazole, isothiazole, pyrazole, pyrrole, imidazole, indole, benzimidazole, quinoline, isoquinoline, furan, benzofuran, benzothiophene, benzothiazole, indazole, thiazole, isoxazole, oxadiazole, indan;
R1 is a lower alkoxy group alkyl-O— with one to four carbon atoms and preferably one carbon,
R2 is an R1 group or hydrogen, an OH or an NH₂ group,
Q is selected from the group consisting of 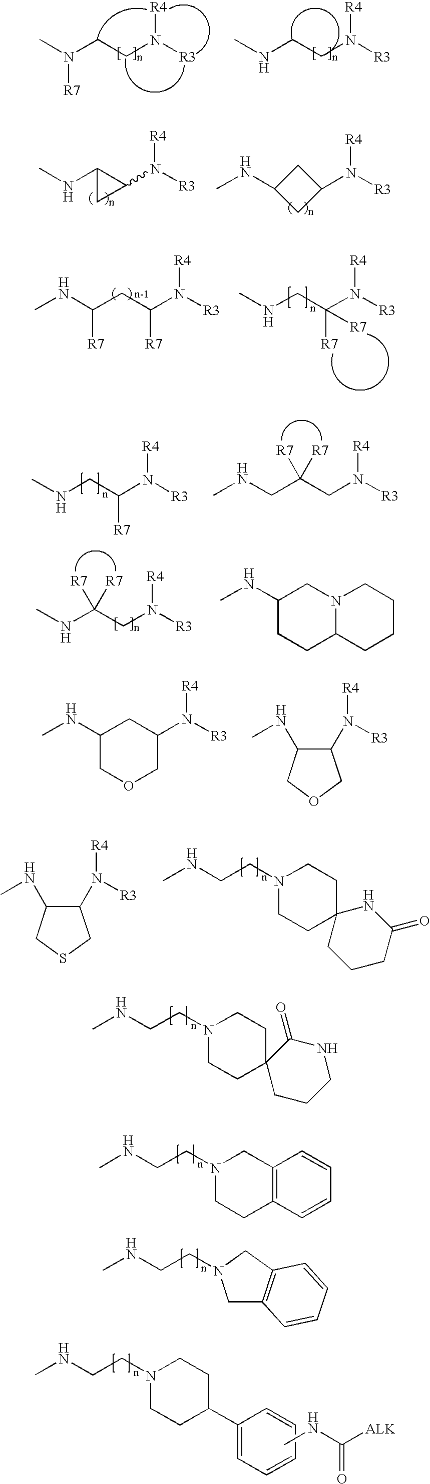 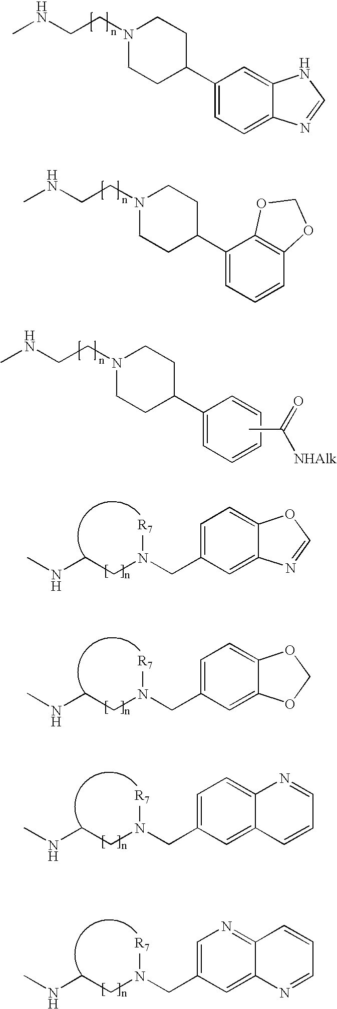
R3 and R4 are the same or different selected from straight or branched alkyl, alkenyl or alkynyl groups with 1-8 carbon atoms; cycloalkyl groups with 3-7 carbon atoms; alkylcycloalkyl with 4-9 carbon atoms; alkylaryl groups such as benzyl, 2-ethylphenyl, 3-propylphenyl, 4-butylphenyl; alkylheterocyclyl groups such as 2-ethylpiperazine, 3-propylpiperidine; alkylheteroaryl groups; the aryl, heterocyclyl and heteroaryl groups may be substituted with substituents such as Alk-CONH—, Alk-O—, HO—, NC—, AlkNH—, Alk₂N—, —CONH₂, —CONHAlk, —CONAlk₂, aryl, substituted aryl, benzyl, substituted benzyl groups
Alk is the same or a different alkyl, alkenyl or alkynyl group;
R3 and R4 may optionally be linked to each other, when possible, as indicated in Formula I; and oxygen or nitrogen atoms may be inserted in the chain or ring in a chemically stable position;
R5 is selected from hydrogen, halogen atoms, alkoxy groups (AlkO—), hydroxy, alkylamino groups (AlkNH—), dialkylamino groups (Alk₂N—), hydroxylalkyl groups, carboxamido groups (—CONH₂, —CONHAlk, —CONAlk₂), acylamido groups (—NHCO-Alk), acyl groups (—CO-Alk), —CHO, nitrile, alkyl, alkenyl or alkynyl groups, —SCH₃, partially or fully fluorinated alkyl, alkoxy or thioalkoxy groups such as —CH₂CF₃, —CF₂CF₃, —CF₃, —OCF₃, —SCF₃; —SO₂NH₂, —SO₂NHAlk, —SO₂NAlk₂, —SO₂Alk;
more than one R5 group, same or different, may be present on Ar₁; when more than one R5 or when one R5 and one R8 group are present they could be connected to each other, directly or with a suitable connecting moiety, to form rings;
X being the same or different H, F, Cl, Br, I, —SCH₃, —CF₃, —OCF₃, —SCF₃, OCH₃, or lower alkyl or alkenyl group;
n is 1, 2 or 3,
R8 is halogen atoms, alkyl, alkenyl or alkynyl groups, cycloalkyl groups with 3-7 carbons, aryl groups (Ar), heteroaryl groups, heterocyclyl groups, alkylcycloalkyl groups, alkylaryl groups, alkylheterocyclyl groups, alkylheteroaryl groups, arylalkoxy groups (e.g. ArCH₂O—), aryloxy groups (ArO—), alkoxy groups (AlkO—), dialkylamino groups (Alk₂N—), —CONHAlk, —CONHAr —CONAlk₂, —NHCO-Alk, —NHCO—Ar, —CO-Alk, —CO—Ar, —SCH₃, partially or fully fluorinated alkyl, alkoxy or thioalkoxy groups such as —CH₂CF₃, —CF₂CF₃, —CF₃, —OCF₃, —SCF₃;
or R8 has the structure 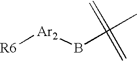
in which B is a single bond or a connecting moiety selected from the group consisting of: 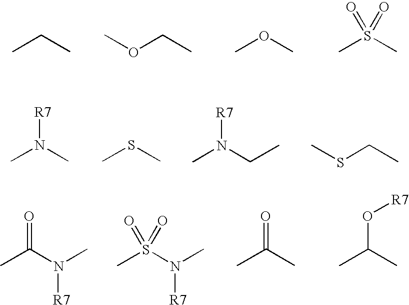
which may be attached via either of the two free bonds to the Ar₁ group;
Ar₂ is an aryl or heteroaryl group such as e.g. phenyl, pyridine, pyrimidine, pyrazine, thiophene, oxazole, isothiazole, pyrazole, pyrrole, imidazole, indole, benzimidazole, quinoline, isoquinoline, furan, benzofuran, benzothiophene, benzothiazole, indazole, thiazole, isoxazole, oxadiazole, indan;
R6 is selected from hydrogen, halogen atoms, alkoxy groups (AlkO—), hydroxy, alkylamino groups (AlkNH—), dialkylamino groups (Alk₂N—), hydroxylalkyl groups, carboxamido groups (—CONH₂, —CONHAlk, —CONAlk₂), acylamido groups (—NHCO-Alk), acyl groups (—CO-Alk), —CHO, nitrile, alkyl, alkenyl or alkynyl groups, —SCH₃, partially or fully fluorinated alkyl, alkoxy or thioalkoxy groups such as —CH₂CF₃, —CF₂CF₃, —CF₃, —OCF₃, —SCF₃, —SO₂NH₂, —SO₂NHAlk, —SO₂NAlk₂, —SO₂Alk;
more than one R6 group, same or different, may be present on Ar₂; when more than one R6 group is present they could be connected to each other to form rings.

In a specific embodiments Q is

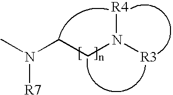

In another specific embodiment of the invention, R8 is 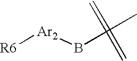
or, alternatively, R8 is selected from halogen atoms, alkyl, alkenyl or alkynyl groups, cycloalkyl groups with 3-7 carbons, aryl groups (Ar), heteroaryl groups, heterocyclyl groups, alkylcycloalkyl groups, alkylaryl groups, alkylheterocyclyl groups, alkylheteroaryl groups, arylalkoxy groups (e.g. ArCH₂O—), aryloxy groups (ArO—), alkoxy groups (AlkO—), dialkylamino groups (Alk₂N—), —CONHAlk, —CONHAr —CONAlk₂, —NHCO-Alk, —NHCO—Ar, —CO-Alk, —CO—Ar, —CF₃, —OCF₃, —SCF₃, SCH₃.

In further embodiments of the invention A is selected from the group consisting of: 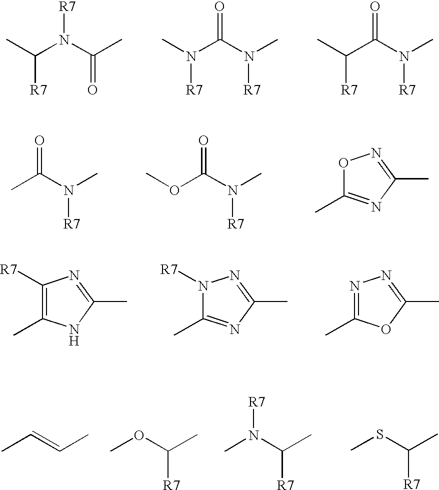
wherein R7 is as defined herein.

More specifically, A may be selected from the group consisting of: 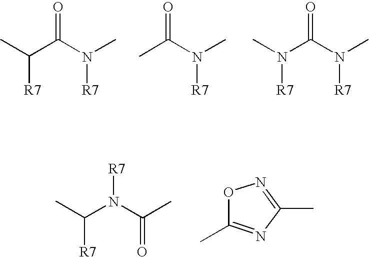
wherein R7 is as defined herein.

In those embodiments wherein B is present, it is a single bond or selected from the group consisting of: 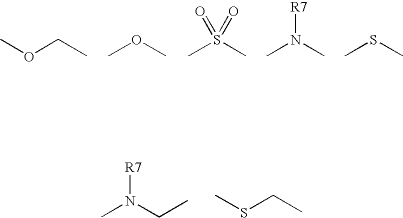
wherein R7 is as defined herein.

Alternatively, B is a single bond or is selected from the group consisting of: 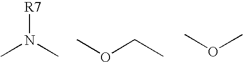
wherein R7 is as defined herein.

The invention also relates to a compound according to formula I with the following structure 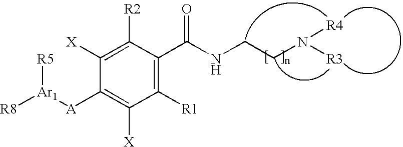
wherein Ar₁, Ar₂, A, B, R1, R2, R3, R4, R5, R6, R7, R8, X and n are as defined herein In this embodiment interesting compounds are those, wherein R8 is

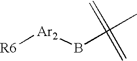

Normally, the —B— moiety is not placed ortho to the -A- linker.

The invention also relates to a compound, wherein Ar₁ and Ar₂ are the same or different aryl or heteroaryl groups such as, e.g., phenyl, pyridine, thiophene, R2 may be hydrogen and/or X may be H, F, Cl, Br, I, CF₃, OCF₃, SCF₃, SCH₃ or lower alkyl or alkenyl group.

In another embodiment, R2 is H and X is H or F; R5 and R6 may be the same or different selected from hydrogen, halogen atoms, alkoxy groups (AlkO—), alkyamino groups (AlkNH—), dialkylamino groups (Alk₂N—), carboxamido groups (—CONH₂, —CONHAlk, CONAlk₂), acylamido groups (—NHCO-Alk), nitrile, lower alkyl groups, —CF₃, —OCF₃, —SCF₃, —SCH₃.

Other compounds according to the invention have the following formulas:

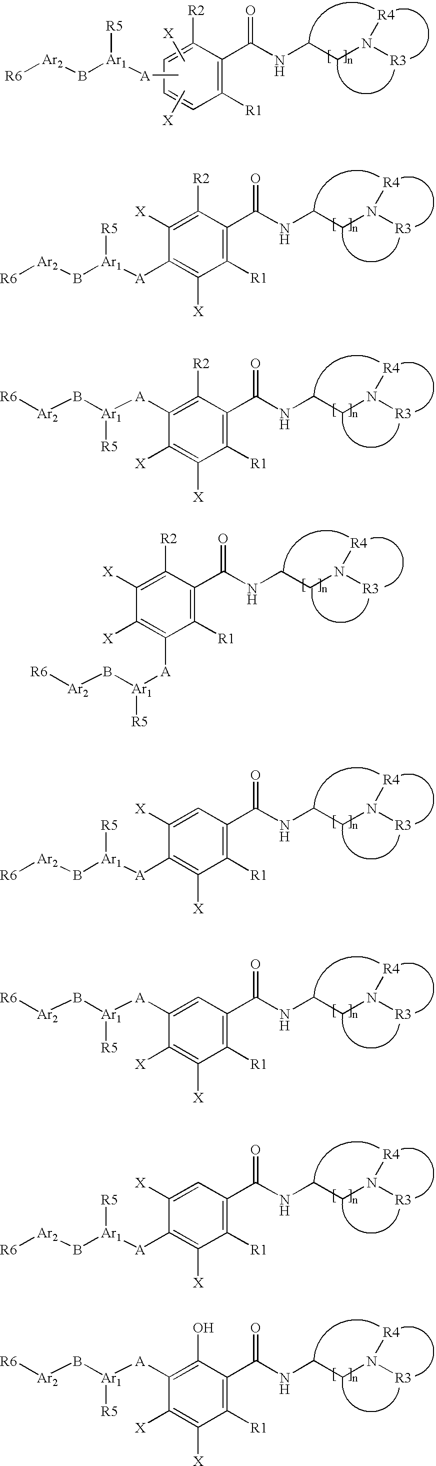

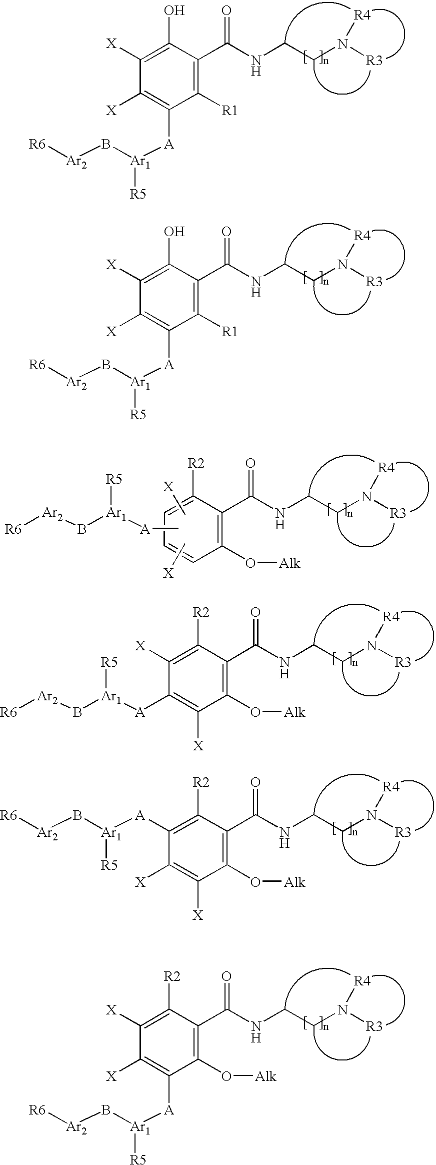

Other specific embodiments appear from the appended claims and the examples herein.

Synthetic Routes

Synthetic Routes

Compounds of formula I are preferably made by connecting an appropriately functionalised (A″) benzamide moiety III with a suitably functionalised (A′) diaryl moiety II using well-known synthetic routes according to the following general scheme (Route 1):

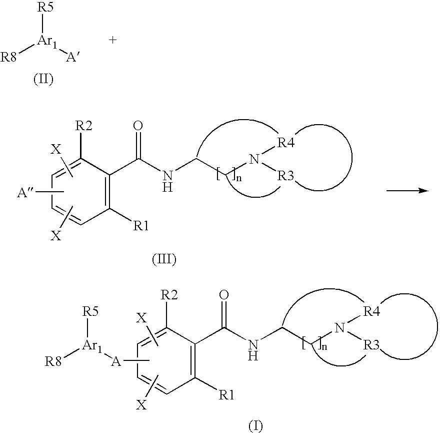

For example, urea bonds -A- can be formed by reaction of II having A′ as isocyanate with III having A″ equal to NH—R7 using appropriate catalysis by base or acid. The reverse use of III having A″ as isocyanate with II having A′ equal to NH—R7 can also be applied. Analogously, carbamates can for example be made by reaction of II having A′ as isocyanate with III having A″ equal to OH or the reverse use of OH and isocyanate in A′ and A″.

Preparation of Amide and Sulphonamide Bonds 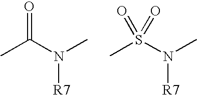
in the connecting A-linkage can be made via reaction of A″ in compound III being NH—R7 with activated forms, e.g. acid chlorides or active esters, of A′ in compound II being COOH or SO₂OH. Alternatively, the conversion can be made directly with the acids having A′ as COOH using suitable coupling reagents such as dicyclohexylcarbodiimide (DCC), and promoters such as 1-hydroxybenzotriazole. The reverse use of A′ and A″ in II and III can be applied as well to form the linker in the opposite direction.

Formation of the connecting A-linkage to form 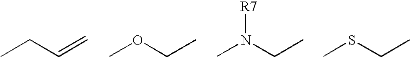
bonds in either direction between Ar1 and the benzamide can be made by N-, O- or S-alkylations of compound II with A′ being OH, NH—R7, or SH with compound III with A″ being a CH₂-L wherein L being a suitable leaving group such as halogen (Cl, Br, I), tosyl or mesyl using appropriate catalysts and conditions. The alkene linkage can be made by a Wittig reaction with compound II with A′ being CHO and compound III with A″ being CH₂—PPh₃. The reverse use of A′ and A″ in II and III can be applied as well to form the linker in the opposite direction.

The 5-membered heterocyclic linkers 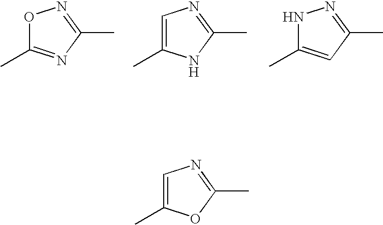
can be made according to standard cyclisation procedures using appropriate solvents, catalysts and temperatures. For example, formation of 1,2,4-triazole can be made from II with A′ being acylhydrazide with III with A″ being amide or thioamide or the reverse orientation of A′ and A″. 1,2,4-Oxadiazole can be formed from II with A′ being amidoxime with III with A″ being carboxylic ester or the reverse orientation of A′ and A″. 1,3,4-Oxadiazole can be formed from II with A′ being acylhydrazide with III with A″ being carboxylic ester or the reverse orientation of A′ and A″.

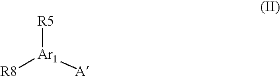

Aromatic substituents R4, R5 and R8 are preferably introduced prior to formation of the A- or B-linkage either direct or via a masked functionality that is compatible with the subsequent synthetic steps.

Alternatively, compounds of formula I are made by connecting an appropriately functionalised (B″) arylated benzamide moiety V with a suitably functionalised (B′) aryl moiety IV using well-known synthetic routes according to the following general scheme (Route 2):

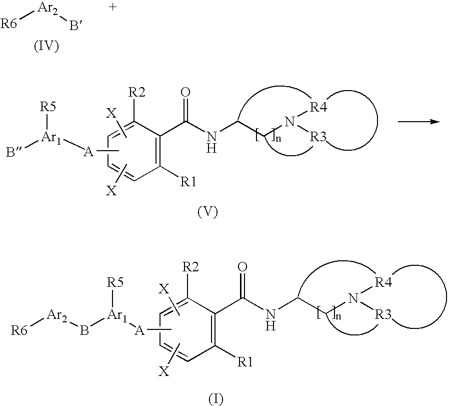

Thus, formation of the connecting B-linkage to form 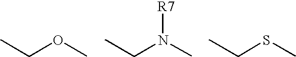
bonds in either direction between Ar1 and Ar2 can be made by N-, O- or S-alkylations of compounds IV having B′ as OH, NH—R7, or SH with compounds V having B″ as CH₂-L, wherein L is a suitable leaving group such as halogen (Cl, Br, I), tosyl or mesyl using appropriate catalysts and conditions. The reverse use of B′ and B″ in IV and V can be applied as well to form the linker in the opposite direction.

Formation of the connecting B-linkage to form 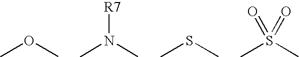
bonds can be made via coupling reactions of compounds IV with B′ being OH, NH—R7, or SH with compound V having B″ as a suitable metal-reagent capable of forming the bond using appropriate catalysts and conditions or with B″ being a halogen that can be replaced under thermal or metal-catalysed conditions. The reverse use of B′ and B″ in IV and V can be applied as well. The —SO₂— linkage may be obtained by oxidation of the corresponding —S— derivative.

Formation of the connecting B-linkage to form 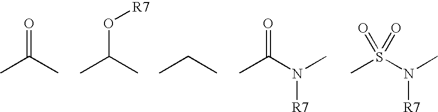
can be made by Friedel-Craft chemistry utilising compounds IV having B′ as e.g. CO—Cl and compounds V having B″ as hydrogen to form the —CO— linkage followed by reduction to —CH(OH)—, that can be alkylated to give —CH(OAlk), or complete reduction to —CH₂—. The amide bond is made according to standard reactions involving compounds IV having B′ as NH—R7 and activated derivatives of compound V with B″ being COOH or coupling reagents and promotors. The reverse use of B′ and B″ in IV and V can be applied as well. The sulphonamides are made analogously from the corresponding SO₂—Cl derivatives and NH—R7 derivatives.

Notably, the —B— linkage is preferably introduced during the synthesis of intermediates II that are used in the coupling with III according to Route 1. In most cases the —B— linkage is made in compounds having A′ groups that are compatible with the reaction conditions and that can be transformed into the required reactive moieties for subsequently forming the -A- linkage.

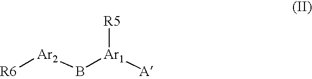

Aromatic substituents R4, R5 and R6 are preferably introduced prior to formation of the A- or B-linkage either direct or via a masked functionality that is compatible with the subsequent synthetic steps.

Compounds of formula I are also obtained by connecting carboxylic acid derivatives VI with amines VII using well-known synthetic routes according to the following general scheme (Route 3):

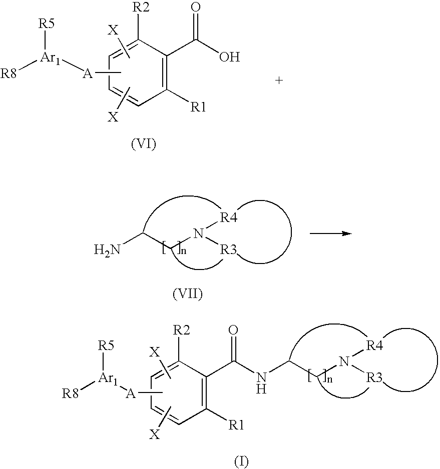

Thus, the benzamide bond is formed by reacting a suitably activated carboxylic acid VI (e.g. acid chloride) with the corresponding amines VII in the presence of a base or using suitable coupling reagents such as DCC in presence of promoting agents and a suitable base.

Alternatively, compounds of formula I can be made by N-alkylation of compounds of formula I having R3 and R4 being hydrogen using well-known synthetic routes such as reductive alkylation or alkylation with alkyl halides in case the functionalisation of the molecule is compatible with this type of reactions (Route 4).

Synthetic Method 1A

Thus, compound (Ib) having NHCON—R7 as linker A with R7 defined as hydrogen or lower alkyl or alkenyl group, can be produced, for instance, by the following urea reaction.

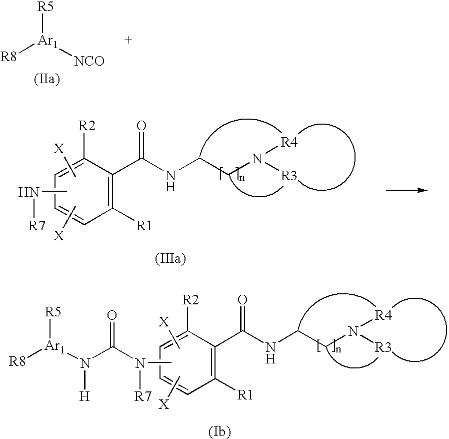

Compound IIa and compound IIIa are reacted in an inert solvent in accordance with standard procedures. Typically, inert solvents can be ether solvents, halogenated hydrocarbon solvents, nitrile solvents and aromatic solvents. Reaction temperature is usually room temperature and the reaction time is 2 hours to 1 day.

Compound IIa can be produced from the corresponding carboxylic acid. For instance, 4-phenoxyphenylisocyanate can be produced in accordance with methods such as described in “Comprehensive Organic Transformation”, 2^{nd} Edition (Wiley); R. C. Larock.

Synthetic Method 1B

Compound Ic having N-AlkCON—R7 as linker A with R7 defined as hydrogen or lower alkyl or alkenyl group, can be produced, for instance, by the following urea reaction.

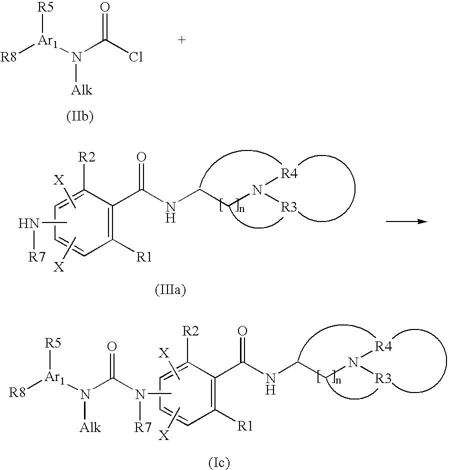

Compound IIIa and 1 equivalent of compound IIb are reacted in an inert solvent in the presence of an excess of a base in accordance with known procedures (e.g. WO 9205174; J. Med. Chem. 43(20), 3653-3664, 2000). Suitable inert solvents can be ether solvents, halogenated hydrocarbon solvents, nitrile solvents and aromatic solvents. As a base can be used for instance triethylamine, diisopropylethylamine and sodium carbonate. Typically, the reaction temperature is 0° C. to room temperature and the reaction time is 1 hour to 1 day.

Compound IIb can be produced from the corresponding N-alkyl aromatic amine by well-known methods. For instance, N-methyl-N-4-phenoxyphenylcarbamoyl chloride can be produced in accordance with methods such as described in J. Labelled Compd. Radiopharma 29(2), 149-155, 1991.

Synthetic Method 1C

Compound If having 5-membered ring urea as linker A can be produced, for instance, by the following reaction sequence.

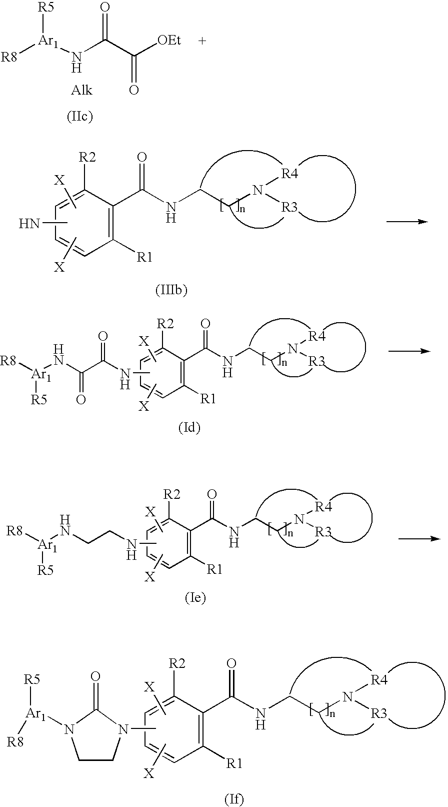

Compound Ie and 1 equivalent of carbonyldiimidazole are reacted in an inert solvent at elevated temperature until the reaction is completed. Typically, the reaction is conducted at reflux in acetonitrile for less than 24 hours.

Compounds IIc, Id and Ie can be produced following the functional group conversions described in procedures like the one in J. Med. Chem. 43(20), 3653-3664, 2000.
Synthetic Method 1D

Compound Ii having CON—R7 as linker A with R7 defined as hydrogen or lower alkyl or alkenyl group, can be produced by the following amidation reaction.

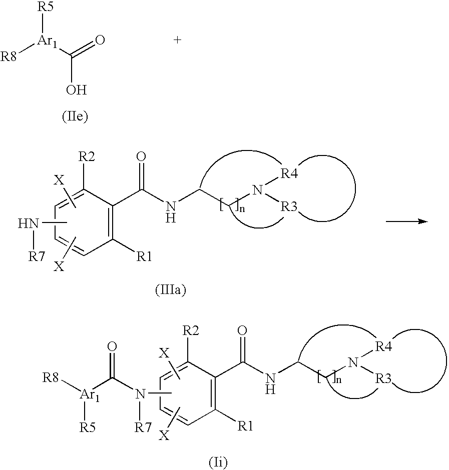

The amide bonds are formed by reacting a suitably activated carboxylic acid lie (acid chloride, mixed anhydrides, esters with phenol bearing electron withdrawing substituents, 1-hydroxybenzotriazole, N-hydroxysuccinimide, 2-hydroxypyridine) with anilines IIIa in an inert solvent in the presence of a base. As inert solvents can be used ether solvents, amide solvents and halogenated hydrocarbon solvents. Suitable bases that can be used are triethylamine, diiisopropylethylamine, pyridine, 4-dimethylaminopyridine (DMAP) and sodium carbonate. The reaction temperature is usually between 0° C. to 30° C. and reaction time is 1 hour to 1 day.

The coupling can also be performed directly from lie using suitable coupling reagents such as dicyclohexylcarbodiimide (DCC), 1-(3-dimethylaminopropyl)-3-ethyl-cabodiimide (EDCI), N-ethoxycarbonyl-2-ethoxy-1,2-dihydroquinoline (EEDQ) preferably in presence of promoting agents capable of forming an active ester such as 1-hydroxybenzotriazole, N-hydroxysuccinimide, 2-hydroxypyridine in an inert solvent in the presence of a base. As inert solvents can be used ether solvents, amide solvents and halogenated hydrocarbon solvents. Suitable bases that can be used are triethylamine, diiisopropylethylamine, pyridine, N-ethyldiisopropylamine, and 4-methylmorpholine. The reaction temperature is usually between 0° C. to 30° C. and reaction time is 1 hour to 1 day.

Analogously, a sulphonamide group, as the connecting A-linkage to form 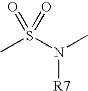
bonds can be made via the corresponding reaction of Ar—NH—R7 (IIIa) with activated forms of sulphonic acids, such sulphonyl chlorides, in the presence of base.
Synthetic Method 2

Compound Ih having 1,2,4-oxadiazole (X═O) or 1,2,4-triazole (X═NH) heterocyclic rings as linker A can be produced, for instance, by the following cyclodehydratation reaction.

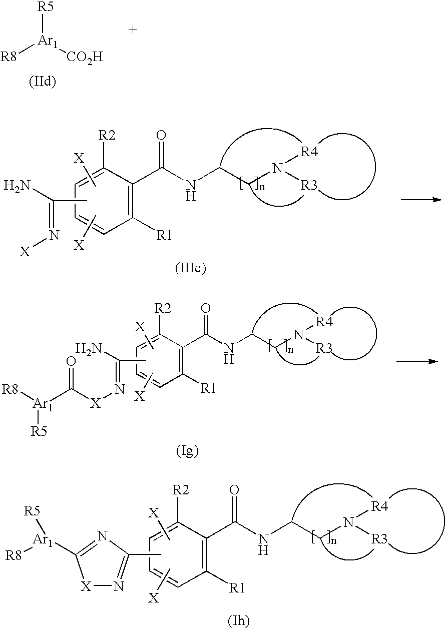

Compound Ig is reacted in an inert solvent with or without the presence of a suitable base or acid (e.g. N-tetrabutyl ammonium fluoride, sodium hydride, sodium ethoxide or polyphosphoric acid) in accordance with standard methods such as described in Tetrahedron Lett. 42, 1441-1443, 2001; Tetrahedron Lett. 42, 1495-1498, 2001. Suitable, inert solvents can be ether solvents, amide solvents and aromatic solvents. The reaction temperature is usually room temperature to 100° C. and the reaction time is 1 hour to 3 days.

Compound Ig can be produced by reacting an activated derivative of compound IId with 1 equivalent of compound IIIc in an inert solvent in the presence of a base. As inert solvents can be used ether solvents, amide solvents and halogenated hydrocarbon solvents. Suitable bases that can be used are triethylamine, diiisopropylethylamine, pyridine and sodium carbonate.

Appropriate examples of the activated derivatives of compound IId include active esters (e.g. esters with phenol bearing electron withdrawing substituents, 1-hydroxybenzotriazole, N-hydroxysuccinamide), acid chlorides, symmetrical or unsymmetrical anhydrides and orthoesters. The reaction temperature is usually between 0° C. to 30° C. and reaction time is 1 hour to 1 day.

Compound IIIc can be produced from the corresponding amino compound IIIb by well known methods such as described in “Comprehensive Organic Transformation”, 2^{nd} Edition (Wiley), R. C. Larock; In “Handbook of Heterocyclic Chemistry”, 2^{nd} Edition (Pergamon), A. R. Katritzky).
Synthetic Method 3

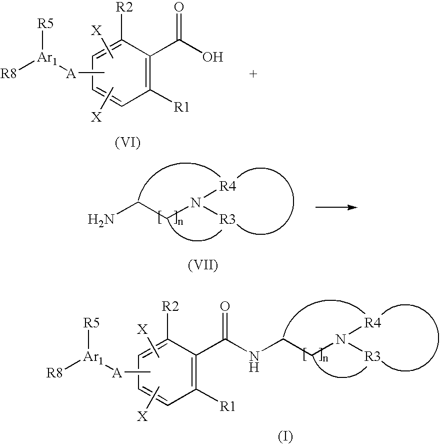

Benzamide bonds are formed by reacting a suitably activated carboxylic acid VI (acid chloride, mixed anhydrides, esters with phenol bearing electron withdrawing substituents, 1-hydroxybenzotriazole, N-hydroxysuccinimide, 2-hydroxypyridine) with the corresponding amines VII in an inert solvent in the presence of a base. As inert solvents can be used ether solvents, amide solvents and halogenated hydrocarbon solvents. Suitable bases that can be used are triethylamine, diiisopropylethylamine, pyridine, 4-dimethylaminopyridine (DMAP) and sodium carbonate. The reaction temperature is usually between 0° C. to 30° C. and reaction time is 1 hour to 1 day.

The coupling can also be performed by using suitable coupling reagents such as dicyclohexylcarbodiimide (DCC), 1-(3-dimethylaminopropyl)-3-ethyl-cabodiimide (EDCI), N-ethoxycarbonyl-2-ethoxy-1,2-dihydroquinoline (EEDQ) preferably in presence of promoting agents capable of forming an active ester such as 1-hydroxybenzotriazole, N-hydroxysuccinimide, 2-hydroxypyridine in an inert solvent in the presence of a base. As inert solvents can be used ether solvents, amide solvents and halogenated hydrocarbon solvents. Suitable bases that can be used are triethylamine, diiisopropylethylamine, pyridine, N-ethyldiisopropylamine, and 4-methylmorpholine. The reaction temperature is usually between 0° C. to 30° C. and reaction time is 1 hour to 1 day.

Synthetic Method 4

Intermediates II 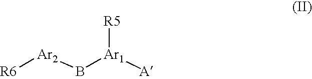
wherein A′ being groups that are compatible with the reaction conditions and that can be transformed into the required reactive moieties for subsequently forming the -A- linkage (e.g. —CO₂H, —NCO, —NAlkCOCl and —NHCOCO₂Alk) can be produced by firstly connecting Ar1 to Ar2 to each other in accordance with standard methods including N-, O- and S-alkylations and metal-catalysed cross couplings. One or several aromatic substituents R5 and R6, depending on their chemical properties, can be introduced either before or after the connection of Ar1 and Ar2 to each other.

Compounds II with B=—O—, —NH—R7-, or —S— are prepared from a suitable aryl halide and the corresponding phenol, aniline or thiol by heating with for example NaH or K₂CO₃ as base with the presence of a copper salt in DMF, pyridine or other high boiling solvents. An example of a metal assisted preparation of diaryl ethers is the coupling of a phenol with an arylbromide in the presence of Pd(OAc)₂ together with a phosphine ligand and K₃PO₄. For instance, 4-(4-chloro-phenoxy)benzoic acid can be produced in a two-steps synthesis from the corresponding 4-fluoro-acetophenone and 4-chlorophenol in accordance with methods such as described in Synthesis, 63-68, 1991 and Eur. J. Med. Chem., 3, 205-214, 1984.

For compounds II with B equal to —CH₂O—, the preparation is performed by heating a benzyl halide and phenol with K₂CO₃ or NaOMe as base. These ethers can also be prepared from suitable benzyl alcohols and phenols utilising Mitsunobu conditions (DEAD and PPh₃). Compounds II with B equal to —CH₂N—R7- can be prepared from an aniline and a benzyl halide using K₂CO₃ as base. The corresponding thioether can be formed from a benzyl halide and thiophenol using KOH or NaOMe as bases and with for example ethanol as the solvent.

When B is equal to —CO— the compounds II can be synthesised from an arylic acid chloride either through a Friedel Craft reaction with an appropriate benzene derivative or via addition of a suitable Grignard reagent. Reduction of the same compound with NaBH₄ gives the compound II with B=—CH(OH)— that can be alkylated to produce —CH(OAlk)-. Utilising hydrogenation with PtO₂ as catalyst or Zn(Hg) as reducing agent yields compounds II with B=—CH₂—.

Compounds II with B=—SO₂— can be prepared from the corresponding sulfide by oxidation with H₂O₂ or KMnO₄.

When B is an amide linkage compounds II can be prepared according to standard protocol from an activated carboxylic acid derivative (acid chloride, mixed anhydrides, esters with phenol bearing electron withdrawing substituents, 1-hydroxybenzotriazole, N-hydroxysuccinimide, 2-hydroxypyridine) and an amine in an inert solvent and in the presence of a base. Suitable bases that can be used are triethylamine, diiisopropylethylamine, pyridine, 4-dimethylaminopyridine (DMAP) and sodium carbonate. The coupling can also be performed by using suitable coupling reagents such as dicyclohexylcarbodiimide (DCC), 1-(3-dimethylaminopropyl)-3-ethyl-cabodimidmide (EDCI), N-ethoxycarbonyl-2-ethoxy-1,2-dihydroquinoline (EEDQ) preferably in presence of promoting agents capable of forming an active ester such as 1-hydroxybenzotriazole, N-hydroxysuccinimide, and 2-hydroxypyridine.

Synthetic Method 5

Intermediate IIIb 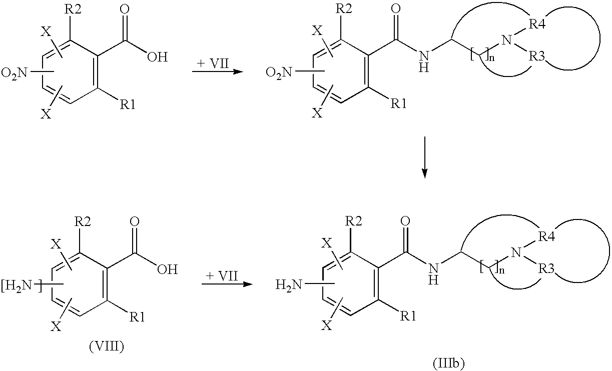
can be prepared by reacting an activated carboxylic acid derivative VIII according to methods described above, preferably having the aniline nitrogen suitably protected (e.g. Boc, CF₃CO), with the corresponding amine VII. The nitrogen may also be masked as a nitro group that subsequently is reduced to form IIIb. The N-alkylated derivative IIIa may be obtained via reductive alkylation of IIIb.

The carboxylic acids VIII are produced by well-known organic reactions including electrophilic substitutions or organometallic reactions such as ortho-lithiation and halogen-metal exchange followed by capture with electrophilic reagents. Alternatively, the aniline nitrogen may be introduced by a benzyne reaction.

Compounds

Below follows some examples of specific compounds according to the invention. In the compounds mentioned, one part of the molecule such as e.g. the amine group, the linker -A-, the linker -B-, the Ar₁ or Ar₂ group, the R4, R5, R6 group or the chain length is varied, while the other parts are conserved. Though not shown nor specifically mentioned, the invention also includes all compounds wherein all variations in one part of the molecule, e.g. linker -A- is combined with all variations in another of the features, e.g. variation in the Ar₁ group.
Variation of the Amine 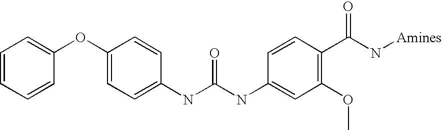
2-Methoxy-4-[3-(4-phenoxy-phenyl)-ureido]-N-(3-pyrrolidin-1-yl-propyl)-benzamide,
N-(4-Dimethylamino-butyl)-2-methoxy-4-[3-(4-phenoxy-phenyl)-ureido]-benzamide,
N-(3-Dimethylamino-2,2-dimethyl-propyl)-2-methoxy-4-[3-(4-phenoxy-phenyl)-ureido]-benzamide,
N-(3-Dipropylamino-propyl)-2-methoxy-4-[3-(4-phenoxy-phenyl)-ureido]-benzamide, 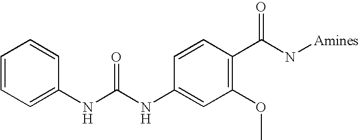
2-Methoxy-4-[3-phenyl-ureido]-N-(3-pyrrolidin-1-yl-propyl)-benzamide,
N-(4-Dimethylamino-butyl)-2-methoxy-4-[3-phenyl-ureido]-benzamide,
N-(3-Dimethylamino-2,2-dimethyl-propyl)-2-methoxy-4-[3-phenyl-ureido]-benzamide,
N-(3-Dipropylamino-propyl)-2-methoxy-4-[3-phenyl-ureido]-benzamide,
   Variation of the Linker A 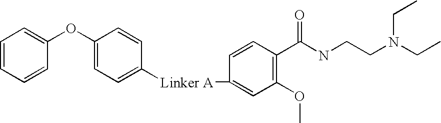
N-(2-Diethylamino-ethyl)-2-methoxy-4-(4-phenoxy-benzoylamino)-benzamide,
N-(2-Diethylamino-ethyl)-2-methoxy-4-[methyl-(4-phenoxy-benzoyl)-amino]-benzamide,
N-(2-Diethylamino-ethyl)-2-methoxy-4-(4-phenoxy-phenylacetylamino)-benzamide,
N-(2-Diethylamino-ethyl)-2-methoxy-4-(α-(4-phenoxy-phenyl)propanoylamino)-benzamide,
N-(2-Diethylamino-ethyl)-2-methoxy-4-(α-(4-phenoxy-phenyl)butanoylamino)-benzamide,
N¹-(2-Diethylamino-ethyl)-2-methoxy-N⁴-(4-phenoxy-phenyl)-terephthalamide,
N¹-(2-Diethylamino-ethyl)-2-methoxy-N⁴-methyl-N⁴-(4-phenoxy-phenyl)-terephthalamide,
N¹-(2-Diethylamino-ethyl)-2-methoxy-N⁴-(4-phenoxy-benzyl)-terephthalamide,
N¹-(2-Diethylamino-ethyl)-2-methoxy-N⁴-methyl-N⁴-(4-phenoxy-benzyl)-terephthalamide,
N-(2-Diethylamino-ethyl)-2-methoxy-4-(4-phenoxy-benzenesulfonylamino)-benzamide,
N-(2-Diethylamino-ethyl)-2-methoxy-4-[methyl-(4-phenoxy-benzenesulfonyl)-amino]-benzamide,
N-(2-Diethylamino-ethyl)-2-methoxy-4-(4-phenoxy-phenylsulfamoyl)-benzamide,
N-(2-Diethylamino-ethyl)-4-[1,3-dimethyl-3-(4-phenoxy-phenyl)-ureido]-2-methoxybenzamide,
N-(2-Diethylamino-ethyl)-2-methoxy-4-[2-oxo-3-(4-phenoxy-phenyl)-imidazolidin-1-yl]-benzamide,
N-(2-Diethylamino-ethyl)-2-methoxy-4-[3-methyl-3-(4-phenoxy-phenyl)-ureido]-benzamide,
N-(2-Diethylamino-ethyl)-2-methoxy-4-[2-oxo-3-(4-phenoxy-phenyl)-tetrahydro-pyrimidin-1-yl]-benzamide,
N-(2-Diethylamino-ethyl)-2-methoxy-4-[5-(4-phenoxy-phenyl)-[1,2,4]oxadiazol-3-yl]-benzamide,
N-(2-Diethylamino-ethyl)-2-methoxy-4-[3-(4-phenoxy-phenyl)-[1,2,4]oxadiazol-5-yl]-benzamide,
N-(2-Diethylamino-ethyl)-2-methoxy-4-[5-(4-phenoxy-phenyl)-4H-imidazol-2-yl]-benzamide,
N-(2-Diethylamino-ethyl)-2-methoxy-4-[5-(4-phenoxy-phenyl)-1H-[1,2,4]triazol-3-yl]-benzamide,
N-(2-Diethylamino-ethyl)-2-methoxy-4-[5-(4-phenoxy-phenyl)-[1,3,4]oxadiazol-2-yl]-benzamide,
N-(2-Diethylamino-ethyl)-2-methoxy-4-[5-(4-phenoxy-phenyl)-2H-[1,2,4]triazol-3-yl]-benzamide,
N-(2-Diethylamino-ethyl)-2-methoxy-4-[2-(4-phenoxy-phenyl)-5H-imidazol-4-yl]-benzamide,
N-(2-Diethylamino-ethyl)-2-methoxy-4-[2-(4-phenoxy-phenyl)-vinyl]-benzamide,
N-(2-Diethylamino-ethyl)-2-methoxy-4-(4-phenoxy-phenoxymethyl)-benzamide,
N-(2-Diethylamino-ethyl)-2-methoxy-4-(4-phenoxy-benzyloxy)-benzamide,
N-(2-Diethylamino-ethyl)-2-methoxy-4-(4-phenoxy-benzylamino)-benzamide,
N-(2-Diethylamino-ethyl)-2-methoxy-4-[methyl-(4-phenoxy-benzyl)-amino]-benzamide,
N-(2-Diethylamino-ethyl)-2-methoxy-4-[(4-phenoxy-phenylamino)-methyl]-benzamide,
N-(2-Diethylamino-ethyl)-2-methoxy-4-{[methyl-(4-phenoxy-phenyl)-amino]-methyl}-benzamide,
N-(2-Diethylamino-ethyl)-2-methoxy-4-(4-phenoxy-phenylsulfanylmethyl)-benzamide,
N-(2-Diethylamino-ethyl)-2-methoxy-4-(4-phenoxy-benzylsulfanyl)-benzamide 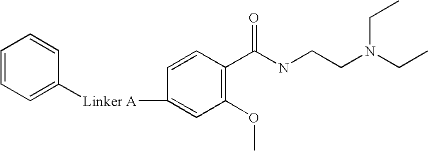
N-(2-Diethylamino-ethyl)-2-methoxy-4-(benzoylamino)-benzamide,
30 N-(2-Diethylamino-ethyl)-2-methoxy-4-[methyl-(benzoyl)-amino]-benzamide,
N-(2-Diethylamino-ethyl)-2-methoxy-4-(phenylacetylamino)-benzamide,
N-(2-Diethylamino-ethyl)-2-methoxy-4-(□-(phenyl)propanoylamino)-benzamide,
N-(2-Diethylamino-ethyl)-2-methoxy-4-(□-(phenyl)butanoylamino)-benzamide,
N¹-(2-Diethylamino-ethyl)-2-methoxy-N⁴-(phenyl)-terephthalamide,
N¹-(2-Diethylamino-ethyl)-2-methoxy-N⁴-methyl-N⁴-(phenyl)-terephthalamide,
N¹-(2-Diethylamino-ethyl)-2-methoxy-N⁴-(benzyl)-terephthalamide,
N¹-(2-Diethylamino-ethyl)-2-methoxy-N⁴-methyl-N⁴-(benzyl)-terephthalamide,
N-(2-Diethylamino-ethyl)-2-methoxy-4-(benzenesulfonylamino)-benzamide,
N-(2-Diethylamino-ethyl)-2-methoxy-4-[methyl-(benzenesulfonyl)-amino]-benzamide,
N-(2-Diethylamino-ethyl)-2-methoxy-4-(phenylsulfamoyl)-benzamide,
N-(2-Diethylamino-ethyl)-4-[1,3-dimethyl-3-(phenyl)-ureido]-2-methoxy-benzamide,
N-(2-Diethylamino-ethyl)-2-methoxy-4-[2-oxo-3-(phenyl)-imidazolidin-1-yl]-benzamide,
N-(2-Diethylamino-ethyl)-2-methoxy-4-[3-methyl-3-(phenyl)-ureido]-benzamide,
N-(2-Diethylamino-ethyl)-2-methoxy-4-[2-oxo-3-(phenyl)-tetrahydro-pyrimidin-1-yl]-benzamide,
N-(2-Diethylamino-ethyl)-2-methoxy-4-[5-(phenyl)-[1,2,4]oxadiazol-3-yl]-benzamide,
N-(2-Diethylamino-ethyl)-2-methoxy-4-[3-(phenyl)-[1,2,4]oxadiazol-5-yl]-benzamide,
N-(2-Diethylamino-ethyl)-2-methoxy-4-[5-(phenyl)-4H-imidazol-2-yl]-benzamide,
N-(2-Diethylamino-ethyl)-2-methoxy-4-[5-(phenyl)-1H-[1,2,4]triazol-3-yl]-benzamide,
N-(2-Diethylamino-ethyl)-2-methoxy-4-[5-(phenyl)-[1,3,4]oxadiazol-2-yl]-benzamide,
N-(2-Diethylamino-ethyl)-2-methoxy-4-[5-(phenyl)-2H-[1,2,4]triazol-3-yl]-benzamide,
N-(2-Diethylamino-ethyl)-2-methoxy-4-[2-(phenyl)-5H-imidazol-4-yl]-benzamide,
N-(2-Diethylamino-ethyl)-2-methoxy-4-[2-(phenyl)-vinyl]-benzamide,
N-(2-Diethylamino-ethyl)-2-methoxy-4-(phenoxymethyl)-benzamide,
N-(2-Diethylamino-ethyl)-2-methoxy-4-(benzyloxy)-benzamide,
N-(2-Diethylamino-ethyl)-2-methoxy-4-(benzylamino)-benzamide,
N-(2-Diethylamino-ethyl)-2-methoxy-4-[methyl-(benzyl)-amino]-benzamide,
N-(2-Diethylamino-ethyl)-2-methoxy-4-[(phenylamino)-methyl]-benzamide,
N-(2-Diethylamino-ethyl)-2-methoxy-4-{[methyl-(phenyl)-amino]-methyl}-benzamide,
N-(2-Diethylamino-ethyl)-2-methoxy-4-(phenylsulfanylmethyl)-benzamide,
N-(2-Diethylamino-ethyl)-2-methoxy-4-(benzylsulfanyl)-benzamide
   Variation of the Linker B 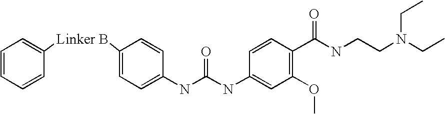
4-[3-(4-Benzyl-phenyl)-ureido]-N-(2-diethylamino-ethyl)-2-methoxy-benzamide,
N-(2-Diethylamino-ethyl)-2-methoxy-4-[3-(4-phenylsulfanyl-phenyl)-ureido]-benzamide,
4-[3-(4-Benzenesulfonyl-phenyl)-ureido]-N-(2-diethylamino-ethyl)-2-methoxy-benzamide,
4-[3-(4-Benzoyl-phenyl)-ureido]-N-(2-diethylamino-ethyl)-2-methoxy-benzamide,
N-(2-Diethylamino-ethyl)-4-{3-[4-(hydroxy-phenyl-methyl)-phenyl]-ureido}-2-methoxybenzamide,
N-(2-Diethylamino-ethyl)-2-methoxy-4-(3-[4-(methoxy-phenyl-methyl)-phenyl]-ureido-benzamide,
N-(2-Diethylamino-ethyl)-2-methoxy-4-[3-(4-phenoxymethyl-phenyl)-ureido]-benzamide,
N-(2-Diethylamino-ethyl)-2-methoxy-4-[3-(4-phenylsulfanylmethyl-phenyl)-ureido]-benzamide,
N-(2-Diethylamino-ethyl)-2-methoxy-4-[3-(4-phenylaminomethyl-phenyl)-ureido]-benzamide,
N-(2-Diethylamino-ethyl)-2-methoxy-4-(3-{4-[(methyl-phenyl-amino)-methyl]-phenyl}-ureido)-benzamide,
4-[3-(4-Benzylamino-phenyl)-ureido]-N-(2-diethylamino-ethyl)-2-methoxy-benzamide,
4-{3-[4-(Benzyl-methyl-amino)-phenyl]-ureido}-N-(2-diethylamino-ethyl)-2-methoxy-benzamide,
4-[3-(4-Benzylsulfanyl-phenyl)-ureido]-N-(2-diethylamino-ethyl)-2-methoxy-benzamide,
4-[3-(4-Benzyloxy-phenyl)-ureido]-N-(2-diethylamino-ethyl)-2-methoxy-benzamide,
4-[3-(4-Benzoylamino-phenyl)-ureido]-N-(2-diethylamino-ethyl)-2-methoxy-benzamide,
4-{3-[4-(Benzoyl-methyl-amino)-phenyl]-ureido}-N-(2-diethylamino-ethyl)-2-methoxy-benzamide,
N-(2-Diethylamino-ethyl)-2-methoxy-4-[3-(4-phenylcarbamoyl-phenyl)-ureido]-benzamide,
N-(2-Diethylamino-ethyl)-2-methoxy-4-{3-[4-(methyl-phenyl-carbamoyl)-phenyl]-ureido}-benzamide,
N-(2-Diethylamino-ethyl)-2-methoxy-4-[3-(4-phenylamino-phenyl)-ureido]-benzamide,
N-(2-Diethylamino-ethyl)-2-methoxy-4-{3-[4-(methyl-phenyl-amino)-phenyl]-ureido}-benzamide.
   Variation of the Aromatic Rings as well as their Substituents 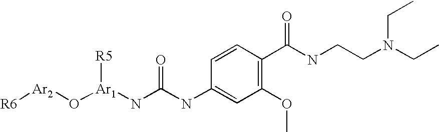
N-(2-Diethylamino-ethyl)-2-methoxy-4-{3-[5-(pyridin-3-yloxy)-pyridin-2-yl]-ureido}-benzamide,
4-(3-[2,2′]Bipyridinyl-6-yl-ureido)-N-(2-diethylamino-ethyl)-2-methoxy-benzamide,
N-(2-Diethylamino-ethyl)-2-methoxy-4-{3-[4-(pyridin-3-yloxy)-phenyl]-ureido}-benzamide,
N-(2-Diethylamino-ethyl)-2-methoxy-4-{3-[4-(pyrimidin-2-yloxy)-phenyl]-ureido}-benzamide,
N-(2-Diethylamino-ethyl)-2-methoxy-4-[3-(2-phenoxy-pyrimidin-5-yl)-ureido]-benzamide,
N-(2-Diethylamino-ethyl)-2-methoxy-4-[3-(5-phenoxy-pyrazin-2-yl)-ureido]-benzamide,
N-(2-Diethylamino-ethyl)-2-methoxy-4-{3-[4-(thiophen-3-yloxy)-phenyl]-ureido}-benzamide,
N-(2-Diethylamino-ethyl)-4-{3-[4-(isothiazol-4-yloxy)-phenyl]-ureido}-2-methoxy-benzamide,
N-(2-Diethylamino-ethyl)-2-methoxy-4-{3-[4-(oxazol-4-yloxy)-phenyl]-ureido}-benzamide,
N-(2-Diethylamino-ethyl)-2-methoxy-4-{3-[4-(1H-pyrazol-4-yloxy)-phenyl]-ureido}-benzamide,
N-(2-Diethylamino-ethyl)-2-methoxy-4-[3-(5-phenoxy-thiophen-3-yl)-ureido]-benzamide,
N-(2-Diethylamino-ethyl)-2-methoxy-4-[3-(2-phenoxy-oxazol-4-yl)-ureido]-benzamide,
N-(2-Diethylamino-ethyl)-2-methoxy-4-[3-(4-phenoxy-oxazol-2-yl)-ureido]-benzamide,
4-{3-[4-(4-Chloro-phenoxy)-phenyl]-ureido}-N-(2-diethylamino-ethyl)-2-methoxy-benzamide,
4-{3-[4-(3,4-Dichloro-phenoxy)-phenyl]-ureido}-N-(2-diethylamino-ethyl)-2-methoxy-benzamide,
4-{3-[4-Fluoro-3-chloro-phenoxy)-phenyl]-ureido}-N-(2-diethylamino-ethyl)-2-methoxy-benzamide,
4-{3-[4-(4-bromo-3-trifluoromethoxy-phenoxy)-phenyl]-ureido}-N-(2-diethylamino-ethyl)-2-methoxy-benzamide,
4-{3-[4-(3,4-methylenedioxy-phenoxy)-phenyl]-ureido}-N-(2-diethylamino-ethyl)-2-methoxy-benzamide,
4-{3-[4-(4-acetamido-phenoxy)-phenyl]-ureido}N-(2-diethylamino-ethyl)-2-methoxy-benzamide,
4-{3-[4-(3-hydroxymethyl-phenoxy)-phenyl]-ureido}-N-(2-diethylamino-ethyl)-2-methoxy-benzamide,
4-{3-[4-(4-trifluoromethyl-phenoxy)-phenyl]-ureido}N-(2-diethylamino-ethyl)-2-methoxy-benzamide,
4-{3-(4-tolyloxy-phenyl)-ureido}-N-(2-diethylamino-ethyl)-2-methoxy-benzamide,
N-(2-Diethylamino-ethyl)-2-methoxy-4-{3-[4-(3-fluoro-4-methoxy-phenoxy)-phenyl]-ureido}-benzamide,
N-(2-Diethylamino-ethyl)-4-{3-[4-(4-hydroxy-phenoxy)-phenyl]-ureido}-2-methoxy-benzamide,
N-(2-Diethylamino-ethyl)-4-{3-[4-(4-dimethylamino-phenoxy)-phenyl]-ureido}-2-methoxy-benzamide,
N-(2-Diethylamino-ethyl)-2-methoxy-4-{3-[4-(4-methylamino-phenoxy)-phenyl]-ureido}-benzamide,
4-{3-[4-(4-Cyano-3-chloro-phenoxy)-phenyl]-ureido}-N-(2-diethylamino-ethyl)-2-methoxy-benzamide,
4-{3-[4-(4-Carbamoyl-phenoxy)-phenyl]-ureido}-N-(2-diethylamino-ethyl)-2-methoxy-benzamide,
4-[3-(3-Chloro-4-phenoxy-phenyl)-ureido]-N-(2-diethylamino-ethyl)-2-methoxy-benzamide,
N-(2-Diethylamino-ethyl)-2-methoxy-4-[3-(2-fluoro-3-methoxy-4-phenoxy-phenyl)-ureido]-benzamide,
N-(2-Diethylamino-ethyl)-2-methoxy-4-[3-(3-bromo-6-methoxy-4-phenoxy-phenyl)-ureido]-benzamide,
N-(2-Diethylamino-ethyl)-2-methoxy-4-[3-(3-methylamino-4-phenoxy-phenyl)-ureido]-benzamide,
N-(2-Diethylamino-ethyl)-2-methoxy-4-[3-(3-hydroxymethyl-4-phenoxy-phenyl)-ureido]-benzamide,
N-(2-Diethylamino-ethyl)-2-methoxy-4-[3-(3-carboxamido-4-phenoxy-phenyl)-ureido]-benzamide,
N-(2-Diethylamino-ethyl)-2-methoxy-4-[3-(3-(N,N-dimethylcarboxamido)-4-phenoxy-phenyl)-ureido]-benzamide,
N-(2-Diethylamino-ethyl)-2-methoxy-4-[3-(3-methyl-4-phenoxy-phenyl)-ureido]-benzamide,
N-(2-Diethylamino-ethyl)-2-methoxy-4-[3-(4-phenoxy-3-trifluoromethoxy-phenyl)-ureido]-benzamide,
N-(2-Diethylamino-ethyl)-2-methoxy-4-[3-(4-phenoxy-2-trifluoromethyl-phenyl)-ureido]-benzamide,
N-(2-Diethylamino-ethyl)-2-methoxy-4-{3-[4-(4-trifluoromethoxy-phenoxy)-phenyl]-ureido}-benzamide 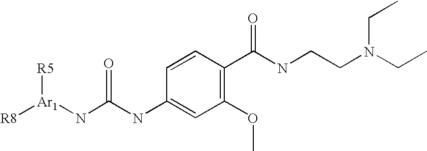
4-[3-(phenyl)-ureido]-N-(2-diethylamino-ethyl)-2-methoxy-benzamide,
N-(2-Diethylamino-ethyl)-2-methoxy-4-[3-(5-indolyl)-ureido]-benzamide,
N-(2-Diethylamino-ethyl)-2-methoxy-4-{3-[3-pyridinyl]-ureido}-benzamide,
4-(3-[2,2′]Bipyridinyl-6-yl-ureido)-N-(2-diethylamino-ethyl)-2-methoxy-benzamide,
N-(2-Diethylamino-ethyl)-2-methoxy-4-{3-[4-(pyridin-3-yloxy)-phenyl]-ureido}-benzamide,
N-(2-Diethylamino-ethyl)-2-methoxy-4-{3-(8-quinolinyl)-ureido}-benzamide,
N-(2-Diethylamino-ethyl)-2-methoxy-4-[3-(2-phenoxy-pyrimidin-5-yl)-ureido]-benzamide,
N-(2-Diethylamino-ethyl)-2-methoxy-4-[3-(5-phenoxy-pyrazin-2-yl)-ureido]-benzamide,
N-(2-Diethylamino-ethyl)-2-methoxy-4-{3-[4-thiophenyl]-ureido}-benzamide,
N-(2-Diethylamino-ethyl)-4-{3-[4-isothiazolyl]-ureido}-2-methoxy-benzamide,
N-(2-Diethylamino-ethyl)-2-methoxy-4-{3-[4-oxazolyl]-ureido}-benzamide,
N-(2-Diethylamino-ethyl)-2-methoxy-4-{3-[4-(1H-pyrazol-4-yloxy)-phenyl]-ureido}-benzamide,
N-(2-Diethylamino-ethyl)-2-methoxy-4-[3-(5-bromo-thiophen-3-yl)-ureido]-benzamide,
N-(2-Diethylamino-ethyl)-2-methoxy-4-[3-(2-chloro-oxazol-4-yl)-ureido]-benzamide,
N-(2-Diethylamino-ethyl)-2-methoxy-4-[3-(4-trifuoromethyl-oxazol-2-yl)-ureido]-benzamide,
4-{3-[4-(4-Chloro-phenoxy)-phenyl]-ureido}-N-(2-diethylamino-ethyl)-2-methoxy-benzamide,
4-{3-[3,4-Dichlorophenyl]-ureido}N-(2-diethylamino-ethyl)-2-methoxy-benzamide,
4-{3-[4-Fluoro-5-chlorothiophen-3-yl]-ureido}-N-(2-diethylamino-ethyl)-2-methoxy-benzamide,
4-{3-[4-bromo-3-trifluoromethoxy-phenyl]-ureido}-N-(2-diethylamino-ethyl)-2-methoxy-benzamide,
4-{3-[5-(3,4-methylenedioxy-phenoxy)-thiopen-3-yl]-ureido}-N-(2-diethylamino-ethyl)-2-methoxy-benzamide,
4-{3-[4-(4-acetamido-phenoxy)-phenyl]-ureido}-N-(2-diethylamino-ethyl)-2-methoxy-benzamide,
4-{3-[4-trifluoromethyl-phenyl]-ureido}-N-(2-diethylamino-ethyl)-2-methoxy-benzamide,
4-{3-(4-methyl-phenyl)-ureido}N-(2-diethylamino-ethyl)-2-methoxy-benzamide,
N-(2-Diethylamino-ethyl)-4-{3-[4-(4-hydroxy-phenoxy)-phenyl]-ureido}-2-methoxy-benzamide,
N-(2-Diethylamino-ethyl)-4-{3-[4-(4-dimethylamino-phenoxy)-phenyl]-ureido}-2-methoxy-benzamide,
N-(2-Diethylamino-ethyl)-2-methoxy-4-{3-[4-(4-methylamino-phenoxy)-phenyl]-ureido}-benzamide,
4-{3-[4-(4-Cyano-3-chloro-phenoxy)-phenyl]-ureido}-N-(2-diethylamino-ethyl)-2-methoxy-benzamide,
4-{3-[4-Carbamoyl-phenyl]-ureido}-N-(2-diethylamino-ethyl)-2-methoxy-benzamide,
4-[3-(3-Chloro-4-cyano-phenyl)-ureido]-N-(2-diethylamino-ethyl)-2-methoxy-benzamide,
N-(2-Diethylamino-ethyl)-2-methoxy-4-[3-(2-fluoro-3-methoxy-4-acetamido-phenyl)-ureido]-benzamide,
N-(2-Diethylamino-ethyl)-2-methoxy-4-[3-(3-bromo-6-methoxy-4-phenoxy-phenyl)-ureido]-benzamide,
N-(2-Diethylamino-ethyl)-2-methoxy-4-[3-(3-hydroxymethyl-4-trifluoromethyl-phenyl)-ureido]-benzamide,
N-(2-Diethylamino-ethyl)-2-methoxy-4-[3-(3-carboxamido-4-iodo-phenyl)-ureido]-benzamide,
N-(2-Diethylamino-ethyl)-2-methoxy-4-[3-(3-(N,N-dimethylcarboxamido)-4-chloro-phenyl)-ureido]-benzamide,
N-(2-Diethylamino-ethyl)-2-methoxy-4-[3-(3-trifluoromethoxy-phenyl)-ureido]-benzamide,
N-(2-Diethylamino-ethyl)-2-methoxy-4-[3-(3-trifluoromethyl-pyridin-2-yl)-ureido]-benzamide,
N-(2-Diethylamino-ethyl)-2-methoxy-4-{3-[4-trifluoromethoxy-thiophen-2-yl]-ureido}-benzamide,
   Substituents on the Benzamide Moiety 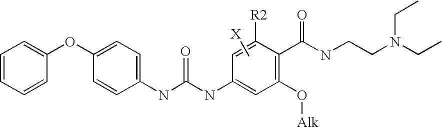
N-(2-Diethylamino-ethyl)-2-ethoxy-4-[3-(4-phenoxy-phenyl)-ureido]-benzamide,
N-(2-Diethylamino-ethyl)-2-hydroxy-6-methoxy-4-[3-(4-phenoxy-phenyl)-ureido]-benzamide,
3-Chloro-N-(2-diethylamino-ethyl)-2-hydroxy-6-methoxy-4-[3-(4-phenoxy-phenyl)-ureido]-benzamide,
3-Bromo-N-(2-diethylamino-ethyl)-2-hydroxy-6-methoxy-4-[3-(4-phenoxy-phenyl)-ureido]-benzamide,
2-Amino-3-chloro-N-(2-diethylamino-ethyl)-6-methoxy-4-[3-(4-phenoxy-phenyl)-ureido]-benzamide,
2-Amino-3-bromo-N-(2-diethylamino-ethyl)-6-methoxy-4-[3-(4-phenoxy-phenyl)-ureido]-benzamide,
2-Amino-N-(2-diethylamino-ethyl)-6-methoxy-4-[3-(4-phenoxy-phenyl)-ureido]-benzamide,
N-(2-Diethylamino-ethyl)-2,6-dimethoxy-4-[3-(4-phenoxy-phenyl)-ureido]-benzamide,
N-(2-Diethylamino-ethyl)-2-hydroxy-6-methoxy-4-[3-(4-phenoxy-phenyl)-ureido]-3-trifluoromethyl-benzamide,
N-(2-Diethylamino-ethyl)-2-hydroxy-6-methoxy-4-[3-(4-phenoxy-phenyl)-ureido]-3-trifluoromethoxy-benzamide 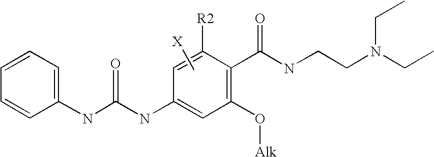
N-(2-Diethylamino-ethyl)-2-ethoxy-4-[3-phenyl-ureido]-benzamide,
N-(2-Diethylamino-ethyl)-2-hydroxy-6-methoxy-4-[3-phenyl-ureido]-benzamide,
3-Chloro-N-(2-diethylamino-ethyl)-2-hydroxy-6-methoxy-4-[3-phenyl-ureido]-benzamide,
3-Bromo-N-(2-diethylamino-ethyl)-2-hydroxy-6-methoxy-4-[3-phenyl-ureido]-benzamide,
2-Amino-3-chloro-N-(2-diethylamino-ethyl)-6-methoxy-4-[3-phenyl-ureido]-benzamide,
2-Amino-3-bromo-N-(2-diethylamino-ethyl)-6-methoxy-4-[3-phenyl-ureido]-benzamide,
2-Amino-N-(2-diethylamino-ethyl)-6-methoxy-4-[3-phenyl-ureido]-benzamide,
N-(2-Diethylamino-ethyl)-2,6-dimethoxy-4-[3-phenyl-ureido]-benzamide,
N-(2-Diethylamino-ethyl)-2-hydroxy-6-methoxy-4-[3-phenyl-ureido]-3-trifluoromethyl-benzamide,
N-(2-Diethylamino-ethyl)-2-hydroxy-6-methoxy-4-[3-phenyl-ureido]-3-trifluoromethoxy-benzamide.
   Salts, Complexes or Solvates

The invention also relates to physiologically acceptable salts, complexes, solvates or prodrugs of the compounds of the invention.

When a compound of the invention possesses a basic functional group it can form a salt with an inorganic or organic acid.

Examples of physiologically acceptable salts of the compounds according to the invention include salts with inorganic acids, salts with organic acids, and salts with basic or acidic amino acids.

Examples of salts with inorganic acids include salts with hydrochloric acid, hydrobromic acid, hydroiodic acid, nitric acid (to form e.g. a nitrate or a nitrite), sulfuric acid (to form e.g., a H₂SO₃ salt, a sulfate or a H₂SO₅ salt) and phosphoric acid (to form e.g. a H₃PO₃ salt or a H₃PO₄ salt)

Examples of salts with organic acids include salts with formic acid, acetic acid, propionic acid, butyric acid, pentanoic acid, oxalic acid, tartaric acid, malonic acid, succinic acid, citric acid, C₄H₈(COOH)₂, C₅H₁₀(COOH)₂, acrylic acid, malic acid, fumaric acid, H₂CO₃, lactic acid, ascorbic acid, benzoic acid, salicylic acid and phthalic acid, trifluoroacetic acid, methanesulfonic acid, benzenesulfonic acid, p-toluenesulfonic acid and 3-chlorobenzoic acid.

Examples of salts with acidic amino acids include salts with aspartic acid and glutamic acid.

Optical Isomers

When a compound of the invention contains optical isomers, diastereomers or other stereroisomers these are included as a compound of the invention as well as the racemate, i.e. mixture of enantiomers. Each of them can be obtained by methods known by a person skilled in the art. For example the optical isomer can be obtained using an optically active synthetic intermediate, an asymmetric synthesis or subjecting the racemic mixture of the final product or a suitable intermediate to optical resolution in accordance with known methods such as, e.g., fractional recrystallisation method, chiral column method, diastereomer method etc.

Other Forms

The invention also encompasses a compound in amorphous, any polymorphous or any crystalline form.

Disorders

The compounds according to the invention can be used in medicine and modulate the activity of a MCH receptor. The compounds may be used as agents for preventing or treating diseases caused by or involving a melanin-concentrating hormone, i.e. they are useful for treating or preventing a MCH or MCH receptor related disorder or abnormality in a subject such as, e.g., an animal or a mammal such as, e.g., a human.

The compounds according to the invention may have antagonistic, inverse agonistic, agonistic or allosteric activity against a MCH receptor, normally antagonistic activity.

In the present context an agonist is defined as a compound that increases the functional activity of a MCH receptor (e.g. the signal transduction through a receptor). The term “agonist” includes partial agonist, i.e. which increases the functional activity of the receptor to a submaximal level. An inverse agonist (or negative antagonist) is defined as a compound that decreases the basal functional activity of a MCH receptor. An allosteric compound is defined as a compound that enhances or diminishes the effects of other receptor ligands.

An antagonist is defined as a compound that decreases the functional activity of a MCH receptor either by inhibiting the action of an agonist or by its own intrinsic activity.

The MCH receptors mentioned in the invention include MCH1 and MCH2 receptors. It also includes MCH receptors having at least about 80% such as, e.g. at least about 85% or at least about 90% homology to the amino acid sequences CTLITAMDAN or CTIITSLDTC.

The MCH receptors may be an animal or a mammalian or non-mammalian receptor, such as a human receptor.

Increasing or decreasing the activity of a MCH receptor such as, e.g. a MCH1 receptor alleviates a MCH-related disorder or abnormality. In specific embodiments the disorder is a steroid or pituitary hormone disorder, an epinephrine release disorder, a gastrointestinal disorder, a cardiovascular disorder, an electrolyte balance disorder, hypertension, diabetes, a respiratory disorder, asthma, a reproductive function disorder, a muscoskeletal disorder, a neuroendocrine disorder, a cognitive disorder, a memory disorder such as, e.g., Alzheimer's disease, a sensory modulation and transmission disorder, a motor coordination disorder, a sensory integration disorder, a motor integration disorder, a dopaminergic function disorder such as, e.g. Parkinson's disease, a sensory transmission disorder, an olfaction disorder, a sympathetic innervation disorder, an affective disorder such as, e.g. depression, a stress-related disorder, a fluid-balance disorder, a urinary disorder such as, e.g., urinary incontinence, a seizure disorder, pain, psychotic behaviour such as, e.g., schizophrenia, morphine or opioid tolerance, opiate addiction or migraine.

More specifically, the compounds of the invention are useful for the treatment or prevention of feeding disorders such as, e.g., overweight, adiposity, obesity and bulimia (e.g. malignant mastocytosis, exogeneous obesity, hyperinsulinar obesity, hyperplasmic obesity, hypophyseal adposity, hypoplasmic obesity, hypophysal adiposity, hypoplasmic obesity, hypothyroid obesity, hypothalamic obesity, symptomatic obesity, infantile obesity, upper body obesity, alimentary obesity, hypogonadal obesity, systemic mastocytosis, simple obesity, central obesity etc.), hyperfagia, emotional disorders, dementia or hormonal disorders.

In the present context the term body mass index or BMI is defined as body weight (kg)/height² (m²), and the term overweight is intended to indicate a BMI in a range from about 25 to about 29.9, whereas obesity is intended to indicate a BMI, which is at least about 30.

A compound of the invention is also useful as an agent for preventing or treating lifestyle diseases such as, e.g., diabetes, diabetic complications (e.g. retinopathy, neuropathy, nephropathy etc.), arteriosclerosis and gonitis.

The present invention further relates to a cosmetic method for reducing overweight and/or for treating of and/or preventing overweight, bulimia, bulimia nervosa, obesity and/or complications thereto, the method comprising administering to an animal such as, e.g. a human in need thereof, an effective amount of a compound according to the invention

The invention also relates to a method for the treatment and/or prophylaxis of diseases caused by a melanin-concentrating hormone, the method comprising administering to a mammal in need thereof an efficient amount of a compound according to the invention.

A mentioned above, the MCH-related disorders may be a feeding disorder. Accordingly, the invention relates to a method for the treatment and/or prophylaxis of diseases caused by feeding disorders, the method comprising administering to a mammal in need thereof an efficient amount of a compound according to the invention.

The invention also relates to a method for modifying the feeding behaviour of a mammal, the method comprising administering to a mammal in need thereof an efficient amount of a compound according to the invention.

Furthermore, the invention relates to a method for the reduction of body mass, the method comprising administering to a mammal in need thereof an efficient amount of a compound according to the invention.

Moreover, the invention relates to a method for the treatment and/or prophylaxis of Syndrome X (metabolic syndrome) or any combination of obesity, insulin resistance, dyslipidemia, impaired glucose tolerance and hypertension, the method comprising administering to a mammal in need thereof an efficient amount of a compound according to the invention.

Another aspect of the invention is a method for the treatment and/or prophylaxis of Type II diabetes or Non Insulin Dependent Diabetes Mellitus (NIDDM), the method comprising administering to a mammal in need thereof an efficient amount of a compound according to the invention.

A still further aspect of the invention is a method for the treatment and/or prophylaxis of bulimia, bulimia nervosa and/or obesity, the method comprising administering to a mammal in need thereof an efficient amount of a compound according to the invention.

Moreover, the invention relates to a method for the treatment and/or prophylaxis of depression and/or anxiety, the method comprising administering to a mammal in need thereof an efficient amount of a compound according to the invention.

Pharmaceutical Compositions

The compounds for use in the methods according to the invention are normally presented in the form of a pharmaceutical or a cosmetic composition comprising the specific compound or a physiologically acceptable salt thereof together with one or more physiologically acceptable excipients.

The compounds may be administered to the animal including a mammal such as, e.g., a human by any convenient administration route such as, e.g., the oral, buccal, nasal, ocular, pulmonary, topical, transdermal, vaginal, rectal, ocular, parenteral (including inter alia subcutaneous, intramuscular, and intravenous), route in a dose that is effective for the individual purposes. A person skilled in the art will know how to chose a suitable administration route.

The pharmaceutical or cosmetic composition comprising a compound according to the invention may be in the form of a solid, semi-solid or fluid composition.

The solid composition may be in the form of tablets such as, e.g. conventional tablets, effervescent tablets, coated tablets, melt tablets or sublingual tablets, pellets, powders, granules, granulates, particulate material, solid dispersions or solid solutions.

A semi-solid form of the composition may be a chewing gum, an ointment, a cream, a liniment, a paste, a gel or a hydrogel.

The fluid form of the composition may be a solution, an emulsion including nano-emulsions, a suspension, a dispersion, a liposomal composition, a spray, a mixture, a syrup or a aerosol.

Fluid compositions, which are sterile solutions or dispersions can utilized by for example intraveneous, intramuscular, intrathecal, epidural, intraperitoneal or subcutaneous injection of infusion. The compounds may also be prepared as a sterile solid composition, which may be dissolved or dispersed before or at the time of administration using e.g. sterile water, saline or other appropriate sterile injectable medium.

Other suitable dosages forms of the pharmaceutical compositions according to the invention may be vagitories, suppositories, plasters, patches, tablets, capsules, sachets, troches, devices etc.

The dosage form may be designed to release the compound freely or in a controlled manner e.g. with respect to tablets by suitable coatings.

The pharmaceutical composition may comprise a therapeutically effective amount of a compound according to the invention.

The content of a compound of the invention in a pharmaceutical composition of the invention is e.g. from about 0.1 to about 100% w/w of the pharmaceutical composition.

The pharmaceutical or cosmetic compositions may be prepared by any of the method well known to a person skilled in pharmaceutical or cosmetic formulation.

In pharmaceutical or cosmetic compositions, the compounds are normally combined with a pharmaceutical excipient, i.e. a therapeutically inert substance or carrier.

The carrier may take a wide variety of forms depending on the desired dosage form and administration route.

The pharmaceutically or cosmetically acceptable excipients may be e.g. fillers, binders, disintegrants, diluents, glidants, solvents, emulsifying agents, suspending agents, stabilizers, enhancers, flavours, colors, pH adjusting agents, retarding agents, wetting agents, surface active agents, preservatives, antioxidants etc. Details can be found in pharmaceutical handbooks such as, e.g., Remington's Pharmaceutical Science or Pharmaceutical Excipient Handbook.

Dosage

Optimal dosages to be administered may be determined by those skilled in the art, and will vary with the particular compound in use, the strength of the composition, the route of administration, the frequency of administration, the age, weight, gender, diet and condition of the subject to be treated and the condition being treated and the advancement of the disease condition etc.

Suitable dosages may be from about 0.001 mg to about 1 g such as, e.g. from about 0.005 to about 750 mg, from about 0.01 to about 500 mg, from about 0.05 to about 500 mg, from about 0.1 to about 250 mg, from about 0.1 to about 100 mg or from about 0.5 to about 50 mg.

The amounts can be divided into one or several doses for administration daily, every second day, weekly, every two weeks, monthly or with any other suitable frequency.

Normally, the administration is daily.

A compound or a pharmaceutical composition according to the invention may be used in combination with other drug substances such as agents for treating disorders like e.g. diabetes, diabetes complications, obesity, hypertension, hyperlipidemia, arteriosclerosis, arthritis, anxiety, and/or depression etc.

Experimental

Materials and Methods

Transfections and Tissue Culture—The cDNA encoding the human MCH-1 receptor was cloned from a human brain cDNA library and cloned into the eukaryotic expression vector pcDNA3.1 (Invitrogen). Assays were performed on transiently transfected COS-7 cells or stably transfected CHO (Chinese Hamster Ovary) cells, expressing the human MCH-1 receptor in pcDNA3.1. Stable MCH-1 receptor transfectants of CHO cells were obtained using 5 μg plasmid cDNA and a standard calcium phosphate transfection method (Johansen et al., 1990; Gether et al., 1992) with subsequent selection in 1 mg/ml G418 (Life Technology). Clones were screened by a MCH receptor radioligand binding assay (as described below). Stably transfected CHO cells were maintained in RPMI 1640 culture medium (Invitrogen), supplemented with 10% fetal calf serum (Invitrogen), 100 U/ml penicillin, 100 μg/ml streptomycin (Life Technology), and 500 μg/ml G418 (Life Technology). COS-7 cells were grown in Dulbecco's modified Eagle's medium (DMEM) 1885 (Invitrogen) supplemented with 10% fetal calf serum, 100 U/ml penicillin, 100 μg/ml streptomycin, and were transiently transfected by a standard calcium phosphate transfection method (Johansen et al., 1990; Gether et al., 1992) two days before assay.

Radioligand Binding Assay—Transiently transfected COS-7 cells or stably transfected CHO cells, expressing human MCH-1 receptor were seeded in multi-well culture plates one day before the assay. The number of cells per well was determined by the apparent expression efficiency of the cell line aiming at 5-10% binding of the added radioligand. Cells were assayed by competition binding for 3 hours at room temperature using 15 μM [¹²⁵I]-MCH (Amersham Pharmacia Biotech) plus variable amounts of unlabeled ligand in 0.5 ml of a 25 mM Hepes buffer, pH 7.4, supplemented with 10 mM MgCl₂, 5 mM MnCl₂, 10 mM NaCl, 0.1% (w/v) bovine serum albumin (BSA), 100 μg/ml bacitracin. The assay was performed in duplicate. Nonspecific binding was determined as the binding in the presence of 1 μM MCH (Bachem). Binding data were analyzed and IC₅₀ values determined by non-linear regression using the Prism software (GraphPad software, San Diego). Values of the dissociation and inhibition constants (K_{d} and Kᵢ) were estimated from competition binding using the equations K_{d}=IC₅₀−L and Kᵢ=IC₅₀/(1+L/K_{d}), respectively, where L is the concentration of radioligand.

Phosphatidylinositol assay—To assay phosphatidylinositol turnover, transiently transfected COS-7 cells or stably transfected CHO cells, expressing human MCH-1 receptor (2×10⁵ cells/well) were incubated for 24 h with 5 μCi of [³H]-myo-inositol (Amersham Pharmacia Biotech) in 0.5 ml inositol-free culture medium. Cells were washed twice in PI-buffer: 20 mM HEPES, pH 7.4, supplemented with 140 mM NaCl, 5 mM KCl, 1 mM MgSO₄, 1 mM CaCl₂, 10 mM glucose, 0.02% (w/v) bovine serum; and were incubated in 0.5 ml PI-buffer supplemented with 10 mM LiCl at 37° C. for 45 min. Phosphatidylinositol turnover was stimulated by submaximal concentrations of MCH, i.e. 10 nM in the presence of increasing amounts of ligand. The ligand was added 5 min. before adding the agonist (MCH). Cells were extracted with 10 mM ice-cold Formic acid, and the generated [³H]-inositol phosphates were purified on Bio-Rad AG 1-X8 anion-exchange resin. Determinations were made in duplicate. PI data were analyzed and IC₅₀ values determined by non-linear regression using the Prism software (GraphPad software, San Diego).

Scintillation Proximity Assay (SPA)—Measurement of [¹²⁵I]-MCHbinding was performed in duplicates by incubating membranes and beads with tracer in the presences of various concentrations of test compounds (10⁻⁸ to 10⁻⁴ M) in DMSO (3 μl) at room temperature for two hours. Membranes and beads were pre-incubated for 20 min. The binding buffer contained 50 mM Tris (pH 7.4), 8 mM MgCl₂, 12% glycerol, 0.1% (w/v) bovine serum albumin (BSA), and protease inhibitors (Complete protease inhibitor cocktail tablets, Roche). A final [¹²⁵I]-MCH(2000 Ci/mmol; Amersham Pharmacia Biotech) concentration of 75.000 cpm/well (33.8 nCi) was applied and PEI-treated WGA-coupled PVT SPA beads, type B from Amersham Pharmacia Biotech were used at a final concentration of 0.4 mg/well. Moreover, CHO-K1 membranes expressing the hMCHreceptor were purchased from Euroscreen (ES-370-M) and a final concentration of 2 μg/well were used. Binding data were analyzed and IC₅₀ values determined by non-linear regression using the Prism software (GraphPad software, San Diego). Values of the inhibition constant (Kᵢ) were estimated from competition binding using the equation Kᵢ=IC₅₀/(−1+L/K_{d}), where L and K_{d} are the concentration and affinity constant, respectively, of the radioligand.

In Vivo model measuring effects on food intake—The effects of test compounds on food intake were studied in male Sprague Dawley rats (290-325 g). The animals were individually housed in plexiglas cages (370 cm²) at room temperature (21±2° C.) and maintained on a 12:12 h light-dark cycle (08.00 h-20.00 h dark). They had free access to water; food (normal rat chow) was only available for the first 6 h of the dark period. The actual experiments studying food intake were conducted when the animals were well accustomed to the housing conditions and feeding paradigm. At least a 10-day acclimatization period was observed after entrance of the animals in the facilities.

At a day of an experiment, weight matched groups (n=6-7) were injected with one of the test compounds (i.p. 10 mg/kg, dissolved in 10% Tween 80), or the solvent (10% Tween 80, 2 ml/kg). Cumulative food intake was registered over the 6-h feeding period. Results were analyzed by one-way ANOVA followed by post hoc Bonferroni test.

### REFERENCES

Gether, U., Marray, T., Schwartz, T. W., and Johansen, T. E. (1992). Stable expression of high affinity NK₁ (substance P) and NK₂ (neurokinin A) receptors but low affinity NK₃ (neurokinin B) receptors in transfected CHO cells. FEBS Lett., 296, 241-244.
Johansen, T. E., Schøller, M. S., Tolstoy, S. and Schwartz, T. W. (1990). Biosynthesis of peptide precursors and protease inhibitors using new constitutive and inducible eukaryotic expressions vectors. FEBS Left., 267, 289-294.

### EXAMPLES

General Comments:

A variety of unsymmetrically amines as in example 77 has been synthesised according to the following literature description, Amundsen, L. H., Sanderson, J. J., Organic Syntheses, Vol. 3, 256 Substitued diarylethers and diarylamines that has been used for urea couplings has been sythesised from arylhalides and phenols (Buck, E., Song, Z. J., Tschaen, D., Dormer, P. G:, Volante, R. P., Reider, P. J., Organic Lett., 2002, 4, 1623) or arylboronic acids and phenols or anilines (Evans, D. A., Katz, J. L., West, T. R., Tetrahedron Lett. 1998, 39, 2937 and Chan, D. M. T., Monaco, K L., Wang, R.-P., Winters, M. P., Tetrahedron Lett., 1998, 39, 2933.).

¹H NMR data are given either in full detailed or with characteristic selected peaks.

LCMS Conditions I: Unpolar solvent: MeCN w/0.01% formic acid. Polar solvent: H₂O w/0.01% formic acid. Gradient: From 20% MeCN to 95% MeCN over 10 min, then 95% MeCN for 5 min. Negative ion scanning mode. Named; an20n15

LCMS Conditions II: Unpolar solvent: MeCN w/0.01% formic acid. Polar solvent: H₂O w/0.01% formic acid. Gradient: From 20% MeCN to 95% MeCN over 10 min, then 95% MeCN for 5 min. Positive ion scanning mode. Named; an20p15

LCMS Conditions III: Unpolar solvent: MeCN w/0.01% formic acid. Polar solvent: H₂O w/0.01% formic acid. Gradient: From 20% MeCN to 95% MeCN over 8 min, then 95% MeCN for 2 min. Positive ion scanning mode. Named; an20p10

LCMS Conditions IV: Unpolar solvent: MeCN w/0.01% formic acid. Polar solvent: H₂O w/0.01% formic acid. Gradient: From 10% MeCN to 95% MeCN over 10 min, then 95% MeCN for 5 min. Positive ion scanning mode. Named; an10p15

### Example 1

### 4-Amino-N-(2-dimethylamino-ethyl)-2-methoxy-benzamide

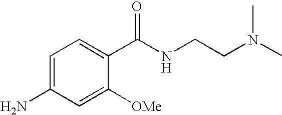

In a flask were placed 4-nitro-2-methoxybenzoic acid (0.50 g, 2.5 mmol) and dichloromethane (10 μl) under nitrogen atmosphere. The solution was cooled to 0° C., whereupon oxalyl chloride (0.20 μl, 2.3 mmol) and N,N′-dimethylformamide (2.0 μl) were added. The reaction mixture was stirred at 0° C. for 30 minutes and at room temperature for 1 h when potassium carbonate (0.25 g, 2.5 mmol) was added followed by addition of N,N-dimethylethylenediamine (0.30 μl, 2.5 mmol). The reaction mixture was stirred overnight before extraction with EtOAc and Na₂SO₄ (aq) was performed. The combined organic phases were dried, filtrated and evaporated leaving 0.54 g (79%) of N-(N,N-dimethylaminoethylamine)-4-nitro-2-methoxybenzamide. ¹H NMR (300 MHz, CDCl₃): δ 2.33 (s, 6H), 2.52-2.60 (m, 2H), 3.52-3.61 (m, 2H), 4.08 (s, 3H), 7.8-7.95 (m, 2H) and 8.29-8.37 (m, 1H).

To a solution of N-(N,N-dimethylaminoethyl)-4-nitro-2-methoxybenzoic amide (0.50 g, 1.87 mmol) in ethanol (10 μl) was Pd/C (40 mg, 20% w/w) added. The reaction mixture was stirred at room temperature under a hydrogen atmosphere over night. The catalyst was filtered off through a pad of celite and the filtrate was concentrated in vacuo. The crude product was chromatographed (Al₂O₃, dichloromethane/methanol/ammonia, 200:10:1) giving 0.42 g (95%) of the title product. ¹H NMR (300 MHz, CDCl₃): δ 2.30 (s, 6H), 2.52 (t, 2H), 3.52 (q, 2H), 3.87 (s, 3H), 6.19 (s, 1H), 6.32 (d, 1H), 7.98 (d, 1H) and 8.13 (brs, 1H).

### Example 2

### Biphenyl-4-carboxylic acid[4-(2-dimethylamino-ethylcarbamoyl)-3-methoxy-phenyl]-amide

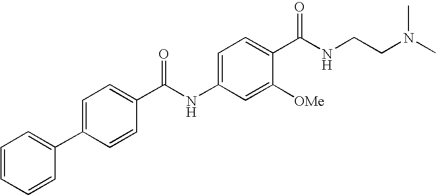

4-phenyl-benzoic acid (0.35 g, 1.8 mmol) was dissolved in dichloromethane (10 μl) in an inert atmosphere and cooled to 0° C., whereupon oxalyl chloride (140 μl, 1.6 mmol) and N,N′-dimethylformamide (5 μl) were added. The reaction mixture was stirred at 0° C. for 30 minutes and at room temperature for 1 h when potassium carbonate (0.25 g, 1.77 mmol) was added. This solution was slowly added under inert atmosphere to Ex 1 dissolved in dichloromethane (5 μl) and the reaction mixture was stirred overnight before extraction with EtOAc and Na2SO4 (aq) was performed. The combined organic phases were dried, filtrated and evaporated. The crude product was chromatographed (Al2O3, dichloromethane/methanol/ammonia, 200:10:1, followed by EtOAc/Heptane, 1:1) giving 10 mg (14%) of the title product. ¹H NMR (300 MHz, CDCl₃): δ 2.40 (s, 6H), 4.04 (s, 3H), 6.96 (d, 1H), 8.36 (br s, 1H).

### Example 3

### Biphenyl-4-carboxylic acid[4-(3-dimethylamino-propylcarbamoyl)-3-methoxy-phenyl]-amide

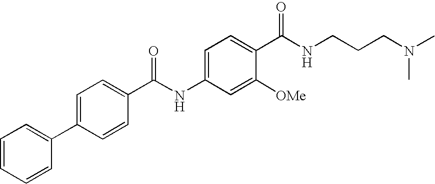

Following the same procedure as described in Ex 1 was N-(N,N-dimethylaminopropyl)-4-amino-2-methoxybenzamide prepared from 2-methoxy-4-nitrobenzoic acid (0.7 g, 3.55 mmol), oxalyl chloride (0.28 μl, 3.2 mmol), triethylamine (0.99 μl, 7.1 mmol) and 3-dimethylaminopropylamine (0.45 μl, 3.55 mmol) followed by reduction with Pd/C (0.04 g, 20% w/w) gave 0.67 g (75%) of N-(N,N-dimethylaminopropyl)-4-amino-2-methoxybenzamide. ¹H NMR (300 MHz, CDCl₃): δ 1.76 (t, 2H), 2.24 (s, 6H), 2.36 (t, 2H), 3.49 (m, 2H), 3.90 (s, 3H), 4.02 (br s, 2H), 6.20 (s, 1H), 6.34 (d, 1H), 7.91 (br s, 1H) and 8.02 (d, 1H).

To a solution of 4-biphenylcarbonyl chloride (0.26 g, 0.80 mmol) in dichloromethane (5 μl) under inert atmosphere was a solution of the above prepared compound in dichloromethane (3 μl) added the reaction mixture was stirred at room temperature for three days. The purification was performed according to the protocol for preparation of Ex 2 and the crude product was chromatographed (Al₂O₃, EtOAc/Heptane, 2:1) giving 0.10 g (30%) of the title product. ¹H NMR (300 MHz, CDCl₃): δ 1.82 (t, 2H), 2.30 (s, 6H), 2.44 (t, 2H), 3.55 (m, 2H), 4.04 (s, 3H), and 6.96 (d, 1H).

### Example 4

### Biphenyl-4-carboxylic acid[4-(2-dimethylamino-ethylcarbamoyl)-phenyl]-amide

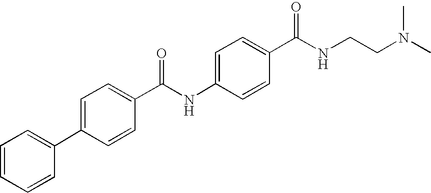

To a solution of 4-nitrobenzoyl chloride (0.50 g, 2.7 mmol) in dichloromethane (10 μl) were triethylamine (0.75 μl, 5.4 mmol) and N,N-dimethylethyldiamine added. The reaction mixture was stirred for three days before extraction with EtOAc and Na₂SO₄ (aq) was performed. The combined organic phases were dried, filtrated and evaporated. The crude product was dissolved in ethanol (10 μl) and Pd/C (40 mg, 20% w/w) was added. The reaction mixture was stirred at room temperature under a hydrogen atmosphere over night. The catalyst was filtered off through a celite pad and the filtrate was concentrated in vacuo giving 0.32 g (56%) of 4-amino-N-(N,N-dimethylaminoethyl)benzamide.

To a solution of 4-biphenylcarbonyl chloride (0.47 g, 2.2 mmol) in dichloromethane (6 μl) under inert atmosphere were added triethylamine (0.4 μl, 2.9 mmol) and 4-amino-N-(N′,N′-dimethylaminoethyl)benzamide (0.3 g, 1.45 mmol) dissolved in dichloromethane (3 μl). The reaction mixture was stirred at room temperature for three days. An additional portion of dichloromethane (3 μl) and PS-trisamine (0.8 g, 3.38 mmol/g) were added to the reaction mixture and the stirring was continued for 2 h at room temperature. The resin was filtered off and rinsed twice with dichloromethane (2×3 μL) before extraction with EtOAc and Na₂SO₄ (aq) was performed. The combined organic phases were dried, filtrated and evaporated. The crude product was chromatographed (Silica, dichloromethane/methanol/ammonia, 100:10:1) and recrystillazed (EtOAc) giving 0.176 g (31%) of the title product. ¹H NMR (300 MHz, CDCl₃): δ 2.25 (s, 6H), 2.74 (t, 2H), 4.19 (t, 2H), 7.90 (d, 2H).

### Example 5

### N-(2-Dimethylamino-ethyl)-2-methoxy-4-(4-phenoxy-benzoylamino)-benzamide

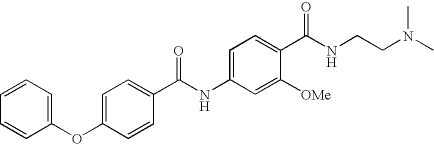

In a flask were placed 4-phenoxy benzoic acid (27 mg, 0.13 mmol) and N,N-dimethylformamide (2 μL) and the flask was cooled to 0° C., whereupon EDAC (24 mg, 0.13 mmol) and HOBt (17 mg, 0.13 mmol) were added. The mixture was gently stirred for 20 minutes at room temperature before Ex 1 (41 mg, 0.19 mmol) dissolved in N,N-dimethylformamide and DIPEA (22 μl, 0.13 mmol) were added. The reaction was continuously stirred three days before extraction with EtOAc and Na₂SO₄ (aq) was performed. The combined organic phases were dried, filtrated and evaporated. The crude product was chromatographed (Silica, dichloromethane/methanol/ammonia, 100:20:2) yielded 12 mg (20%) of the title product. ¹H NMR (300 MHz, CDCl₃): δ 2.48 (s, 6H), 2.77 (m, 2H), 3.68 (m, 2H), 4.03 (s, 3H), 8.16 (d, 1H), and 8.39 (br s, 1H).

### Example 6

### N-(3-Dimethylamino-propyl)-2-methoxy-4-(4-phenoxy-benzoylamino)-benzamide

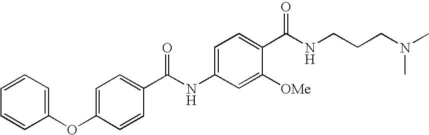

N-(N,N-dimethylaminopropyl)-4-amino-2-methoxybenzamide was prepared according to the experiment described in Ex 3. In a flask were placed PS-DCC (1.2 g, 1.35 mmol/g), dichloromethane (15 μL), 4-phenoxy benzoic acid (0.26 g, 1.2 mmol) and HOBt (0.18 g, 1.35 mmol) and the mixture was gently stirred for 10 minutes before N-(N,N-dimethylaminopropyl)-4-amino-2-methoxybenzamide (0.20 g, 0.80 mmol) was added. The reaction was stirred for three days when PS-trisamine (1.0 g, 3.38 mmol/g) was added. After 2 h the resins were filtered off and rinsed with dichloromethane (20 μL). The solvent was removed under vacuum giving the crude product. Chromatography (Silica, dichloromethane/methanol/ammonia, 200:10:1) yielded 8 mg (2%) of the title product. ¹H NMR (300 MHz, CDCl₃): δ 1.8 (dt, 2H), 2.28 (s, 3H), 2.42 (t, 2H), 3.53 (q, 2H), 4.02 (s, 3H), 6.91 (d, 1H) and 7.89 (d, 2H).

### Example 7

### N-(3-Dimethylamino-propyl)-2-methoxy-4-(3-phenoxy-benzoylamino)-benzamide

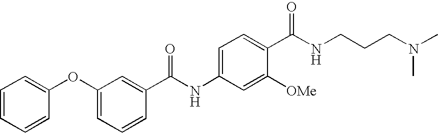

N-(N,N)-dimethylaminopropyl)-4-amino-2-methoxybenzamide was prepared according to the experiment described in Ex 3. In a flask were placed PS-DCC (1.2 g, 1.35 mmol/g), dichloromethane (15 μL), 3-phenoxybenzoic acid (0.26 g, 1.2 mmol) and HOBt (0.18 g, 1.35 mmol) and the mixture was gently stirred for 10 minutes before N-(N,N-dimethylaminopropyl)-4-amino-2-methoxybenzamide (0.20 g, 0.80 mmol) was added. The reaction was stirred for three days when PS-trisamine (1.0 g, 3.38 mmol/g) was added. After 2 h the resins were filtered off and rinsed with dichloromethane (20 μL). The solvent was removed under vacuum giving the crude product. Chromatography (Silica, dichloromethane/methanol/ammonia, 100:20:2) yielded 11 mg (3%) of the title product. ¹H NMR (300 MHz, CDCl₃): δ 1.80 (dt, 2H), 2.28 (s, 3H), 2.42 (t, 2H), 3.54 (m, 2H), 4.00 (s, 3H), 6.92 (d, 1H) and 7.99 (d, 1H).

### Example 8

### N-(2-Dimethylamino-ethyl)-2-methoxy-4-[3-(4-phenoxy-phenyl)-ureido]-benzamide

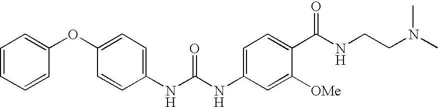

To a solution of Ex 1 (30 mg, 0.13 mmol) in dichloromethane (2 μL) under nitrogen atmosphere was 4-phenoxyphenylisocyanate (64 μl, 0.30 mmol) added. The reaction was stirred for 2 h at room temperature, whereupon PS-trisamine (100 mg, 4.2 mmol/g). The suspension was gentle stirred over night. Methanol (20 μL) was added to dissolve some precipitation before the resin was filtered off and rinsed with dichloromethane (10 μL). The solvents were removed in vacuo and the crude product was purified through chromatography (silica, dichloromethane/methanol/ammonia, 100:20:2) giving 24 mg (42%) of the title compound. ¹H NMR (300 MHz, CDCl₃): δ 2.53 (t, 2H), 3.54 (m, 2H), 3.90 (s, 3H), 8.52 (s, 1H), 8.67 (s, 1H).

### Example 9

### 2-Methoxy-4-[3-(4-phenoxy-phenyl)-ureido]-benzoic acid

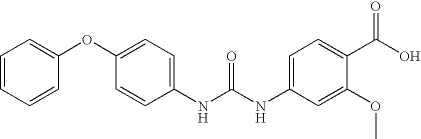

To a solution of 4-nitro-2-methoxybenzoic acid (5.0 g, mmol) in ethanol (100 μL) was added Pd/C (200 mg, 20% w/w). The reaction mixture was stirred at room temperature under a hydrogen atmosphere over night. The catalyst was filtered off through a pad of celite and the filtrate was concentrated in vacuo giving 4-amino-2-methoxybenzoic acid.

To a solution of 4-amino-2-methoxybenzoic acid (0.50 g, 3.0 mmol) in dichloromethane (10 μL) was added 4-phenoxyphenylisocyanate (0.65 μL, 3.6 mmol) under inert atmosphere. The reaction mixture was stirred for three days at room temperature and a precipitate was formed. Filtration gave 1.1 g (97%) of the title compound. ¹H NMR (300 MHz, CDCl₃): δ 3.79 (s, 3H), 6.92-7.02 (m, 5H), 7.09 (t, 1H), 7.32-7.42 (m, 3H), 7.48 (d, 2H), 7.66 (d, 1H), 8.79 (s, 1H), and 9.03 (s, 1H).

### Example 10

### N-(1-Ethyl-pyrrolidin-2-ylmethyl)-2-methoxy-4-[3-(4-phenoxy-phenyl)-ureido]-benzamide

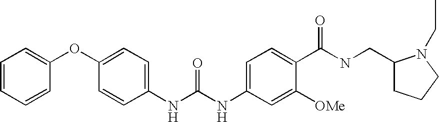

In a flask were placed Ex 9 (57 mg, 0.15 mmol), HOBt (23 mg, 0.17 mmol), PS-DCC (0.15 g, 1.35 mmol/g), and dichloromethane (2 μL). The mixture was stirred at room temperature for 30 minutes, whereupon 2-aminomethyl-ethylpyrrolidine (0.10 mmol) was added. The reaction mixture was stirred over night. PS-trisamine (140 mg, 0.50 mmol) was added and stirring was continued for a day more. The resin was filtered off and rinsed with dichloromethane (3×2 μL). The solvent was removed in vacuo giving 35 mg (71%) of the title product. ¹H NMR (300 MHz, CDCl₃): δ 1.14 (t, 3H), 3.88 (s, 3H), 6.69 (d, 1H), 8.63 (t, 1H), 8.82 (s, 1H), 9.12 (s, 1H).

### Example 11-18

According to the procedure outlined in example 10 were the following compounds prepared utilizing Ex 9 and the corresponding primary amines to the R-group;

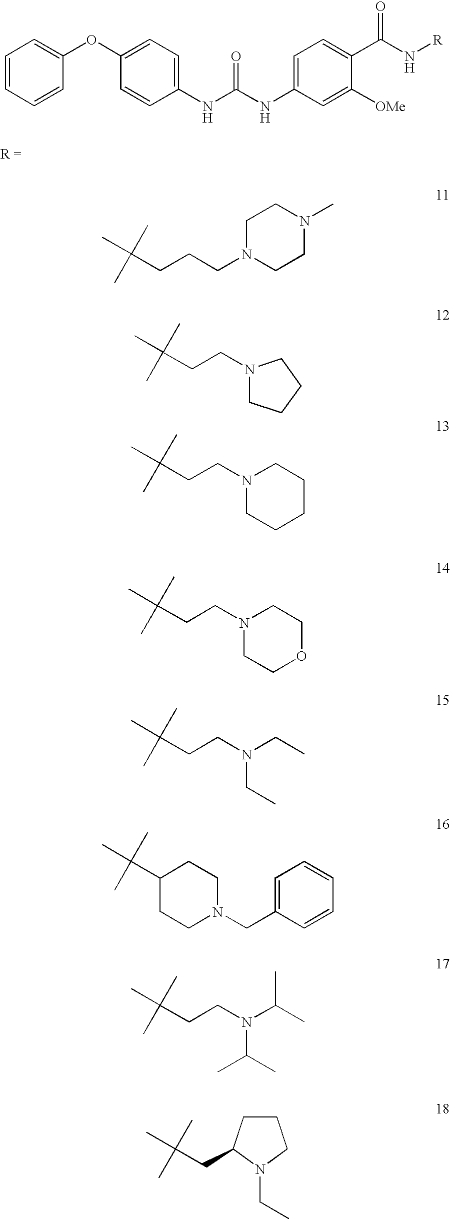

### Example 11

### 2-Methoxy-N-[3-(4-methyl-piperazin-1-yl)-propyl]-4-[3-(4-phenoxy-phenyl)-ureido]-benzamide

Ex 9 and 1-(3-aminopropyl)-4-methylpiperazine was coupled giving 43 mg (82%) of the title product. ¹H NMR (300 MHz, CDCl₃): δ 3.91 (s, 3H), 6.60 (d, 1H), 8.69 (s, 1H), 9.02 (s, 1H).

### Example 12

### 2-Methoxy-4-[3-(4-phenoxy-phenyl)-ureido]-N-(2-pyrrolidin-1-yl-ethyl)-benzamide

Ex 9 and N-(2-aminoethyl)pyrrolidine was coupled giving 30 mg (63%) of the title product. ¹H NMR (300 MHz, CDCl₃): δ 3.82 (s, 3H), 6.72 (d, 1H), 8.44 (t, 1H), 8.85 (s, 1H), 9.13 (s, 1H).

### Example 13

### 2-Methoxy-4-[3-(4-phenoxy-phenyl)-ureido]-N-(2-piperidin-1-yl-ethyl)-benzamide

Ex 9 and 1-(2-aminoethyl)piperidine was coupled giving 40 mg (81%) of the title product. ¹H NMR (300 MHz, CDCl₃): δ 3.91 (s, 3H), 6.64 (d, 1H), 7.06 (t, 1H), 7.95 (d, 1H), 8.58 (t, 1H), 8.76 (s, 1H), 9.03 (s, 1H).

### Example 14

### 2-Methoxy-N-(2-morpholin-4-yl-ethyl)-4-[3-(4-phenoxy-phenyl)-ureido]-benzamide

Ex 9 and 4-(2-aminoethyl)morpholine was coupled giving 18 mg (36%) of the title product. ¹H NMR (300 MHz, CDCl₃): δ 3.99 (s, 3H), 6.47 (d, 1H), 7.08 (t, 1H), 8.47 (s, 1H), 8.58 (t, 1H), 8.74 (s, 1H).

### Example 15

### N-(2-Diethylamino-ethyl)-2-methoxy-4-[3-(4-phenoxy-phenyl)-ureido]-benzamide

Ex 9 and N,N-diethyl-ethylendiamine was coupled giving 38 mg (78%) of the title product. ¹H NMR (300 MHz, CDCl₃): δ 1.09 (t, 6H), 2.70 (q, 4H), 3.86 (s, 3H), 6.73 (d, 1H), 7.05 (t, 1H), 8.55 (t, 1H), 8.89 (s, 1H), 9.19 (s, 1H).

### Example 16

### N-(1-Benzyl-piperidin-4-yl)-2-methoxy-4-[3-(4-phenoxy-phenyl)-ureido]-benzamide

Ex 9 and 4-amino-1-benzylpiperidine was coupled giving 39 mg (70%) of the title product. ¹H NMR (300 MHz, CDCl₃): δ 3.49 (s, 2H), 3.91 (s, 3H), 8.51 (s, 1H), 8.76 (s, 1H).

### Example 17

### N-(2-Diisopropylamino-ethyl)-2-methoxy-4-[3-(4-phenoxy-phenyl)-ureido]-benzamide

¹H NMR (300 MHz, CDCl₃): δ 3.75 (s, 3H), 8.82 (s, 1H), 9.09 (s, 1H).

### Example 18

### N-(1-Ethyl-pyrrolidin-2R-ylmethyl)-2-methoxy-4-[3-(4-phenoxy-phenyl)-ureido]-benzamide

Ex 9 and (R)-2-aminomethyl-ethylpyrrolidine was coupled giving 35 mg (71%) of the title product. ¹H NMR (300 MHz, CDCl₃): 31.14 (t, 3H), 3.88 (s, 3H), 6.69 (d, 1H), 8.63 (t, 1H), 8.82 (s, 1H), 9.12 (s, 1H).

The following examples were prepared from Ex 9 according to the same procedure as Ex 10-18

### Example 19

### N-(4-Benzyl-morpholin-2-ylmethyl)-2-methoxy-4-[3-(4-phenoxy-phenyl)-ureido]-benzamide

¹H NMR (300 MHz, CDCl₃): δ 3.91 (s, 3H), 6.49 (dd, 1H), 7.96 (d, 1H), 8.42 (t, 1H), 8.56 (s, 1H), 8.82 (s, 1H).

### Example 20

### N-(1-Benzyl-pyrrolidin-3-yl)-2-methoxy-4-[3-(4-phenoxy-phenyl)-ureido]-benzamide

¹H NMR (300 MHz, CDCl₃): δ 3.93 (s, 3H), 6.48 (dd, 1H), 8.40 (d, 1H), 8.60 (s, 1H), 8.87 (s, 1H).

### Example 21

### N-(2-Diethylamino-1-methyl-ethyl)-2-methoxy-4-[3-(4-phenoxy-phenyl)-ureido]-benzamide

¹H NMR (300 MHz, CDCl₃): δ 1.12 (t, 6H), 3.84 (s, 3H), 9.51 (s, 1H), 9.88 (s, 1H).

### Example 22

### N-[2-(4-Benzyl-piperazin-1-yl)-ethyl]-2-methoxy-4-[3-(4-phenoxy-phenyl)-ureido]-benzamide

¹H NMR (300 MHz, CDCl₃): 33.89 (s, 3H), 6.50 (dd, 1H), 8.65-8.70 (m, 2H), 8.56 (s, 1H).

### Example 23

### 2-Methoxy-N-(3-morpholin-4-yl-propyl)-4-[3-(4-phenoxy-phenyl)-ureido]-benzamide

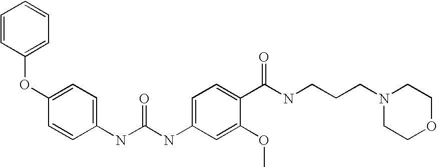

¹H NMR (300 MHz, CDCl₃): δ 1.82(m 2H), 2.42(m 6H), 3.46 (m 2H), 3.54 (m 4H), 3.68 (s 3H), 6.47 (dd 1H), 6.88-7.41 (m 9H), 7.90 (m 2H), 8.22 (m 1H), 8.58 (s 1H), 8.87 (s 1H).

### Example 24

### 2-Methoxy-N-[3-(2-methyl-piperidin-1-yl)-propyl]-4-[3-(4-phenoxy-phenyl)-ureido]-benzamide

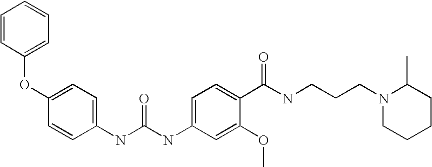

¹H NMR (300 MHz, CDCl₃): δ 1.27 (m 4H), 1.42-4.0(m 14H), 3.86 (s 3H), 6.91-7.83 (m 12H), 8.21 (m 1H), 9.23 (s 1H), 9.61 (s 1H).

### Example 25

### N-(3-Diethylamino-propyl)-2-methoxy-4-[3-(4-phenoxy-phenyl)-ureido]-benzamide

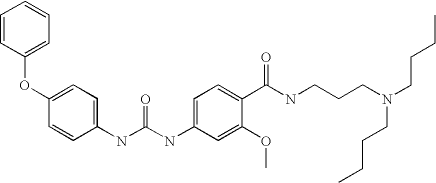

¹H NMR (300 MHz, CDCl₃): δ 0.88 (t 6H), 1.29 (m 4H), 1.59 (m 4H), 1.99 (m 2H), 2.82-2.95 (m 6H), 3.47 (m 2H), 3.87 (s 3H), 6.89-8.23 (m 13H), 9.17 (s H), 9.53 (s 1H).

### Example 26

### 2-Methoxy-N-[2-(1-methyl-pyrrolidin-2-yl)-ethyl]-4-[3-(4-phenoxy-phenyl)-ureido]-benzamide

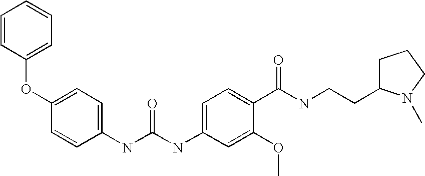

¹H NMR (300 MHz, CDCl₃): δ 1.26-3.60 (m 14H), 3.85 (s 3H), 6.81-8.15 (m 13H), 8.99 (s 1H), 9.45 (s 1H)

### Example 27

### N-(3-Dibutylamino-propyl)-2-methoxy-4-[3-(4-phenoxy-phenyl)-ureido]-benzamide

¹H NMR (300 MHz, CDCl₃): δ 0.88 (t, 6H), 3.87 (t, 3H), 8.21 (t, 1H), 9.17 (s, 1H), 9.53 (s, 1H).

### Example 28

### N-(4-Dimethylamino-phenyl)-2-methoxy-4-[3-(4-phenoxy-phenyl)-ureido]-benzamide

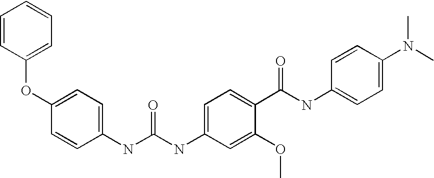

¹H NMR (300 MHz, CDCl₃): 32.85 (s 6H), 4.06 (s 3H), 6.62-7.42 (m 14H), 8.12 (m 3H), 9.00 (s 1H), 9.79 (s 1H).

### Example 29

### N-(3-Dimethylamino-phenyl)-2-methoxy-4-[3-(4-phenoxy-phenyl)-ureido]-benzamide

¹H NMR (300 MHz, CDCl₃): δ 2.85 (s, 6H), 4.08 (s, 3H), 9.90 (s, 1H).

### Example 30

### 2-Methoxy-4-methylamino-benzoic acid methyl ester

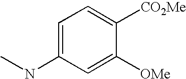

A solution of sodium methoxide (0.745 g, 13.8 mmol), paraformaldehyde (0.124 g, 4.14 mmol) and methyl 4-amino-2-methoxybenzoate (050 g, 2.76 mmol) in methanol (40 μL) was stirred overnight at 40° C. before sodium borohydride (0.229 g, 6.07 mmol) was added at room temperature. The resulting mixture was heated at 50° C. for 8 hours. Methanol was removed in vacuo. The residue was partitioned between saturated aqueous NaHCO3 and dichloromethane. The organic phase was separated and the aqueous phase was extracted with dichloromethane (3×20 mL). The combined organic phases were dried over MgSO₄, filtered and evaporated in vacuo to give a crude solid which was chromatographed over silica gel (CH₂Cl₂/MeOH/NH₃: 9514.5/0.5) to give the title compound as a white solid (0.278 g, 1.43 mmol, 52%). ¹H NMR (300 MHz, CDCl₃): δ 2.88 (s, 3H), 3.82 (s, 3H), 3.87 (s, 3H), 4.30 (bs, 1H), 6.07 (s, 1H), 6.14 (d, 1H), 7.76 (d, 1H)

### Example 31

### 2-Methoxy-4-[1-methyl-3-(4-phenoxy-phenyl)-ureido]-benzoic acid methyl ester

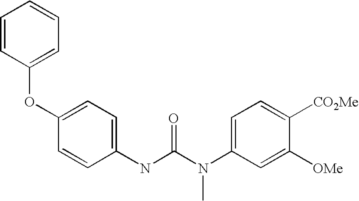

The title compound Ex 31 was obtained by carrying out the same procedure as in Example 8, using Ex 30 and commercially available 4-phenoxyphenylisocyanate. ¹H NMR (300 MHz, CDCl₃): δ 3.36 (s, 3H), 3.91 (s, 6H), 6.35 (s, 1H), 6.93-7.26 (m, 11H), 7.88 (d, 1H)

### Example 32

### 2-Methoxy-4-[1-methyl-3-(4-phenoxy-phenyl)-ureido]-benzoic acid

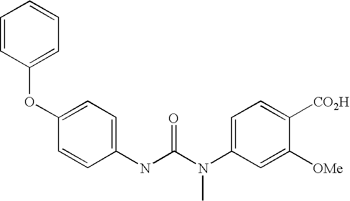

A solution of Ex 31 (0.38 g, 0.93 mmol) and lithium hydroxide (0.034 g, 1.4 mmol) in a THF/water mixture (2/1, 6 μL) was stirred at 30° C. for 3 days. After removal of the solvent in vacuo, the residue was diluted with water and washed with dichloromethane. The aqueous phase was then saturated with solid sodium chloride and acidified to pH=1 with a 6N aq. HCl solution. The aqueous phase was extracted with dichloromethane. The organic phases were combined, washed with brine, dried over MgSO₄ and concentrated in vacuo to give the title compound Ex 32 as a white solid (0.249 g, 0.63 mmol, 68%). ¹H NMR (300 MHz, CDCl₃): δ 3.40 (s, 3H), 4.09 (s, 3H), 6.52 (s, 1H), 6.93-7.33 (m, 11H), 8.20 (d, 1H)

### Example 33

### N-(2-Diethylamino-ethyl)-2-methoxy-4-[1-methyl-3-(4-phenoxy-phenyl)-ureido]-benzamide

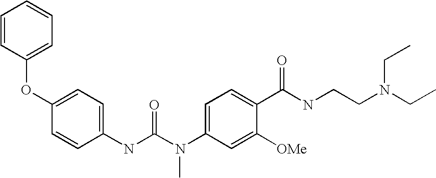

A solution of compound Ex 32 (0.02 g, 0.051 mmol), EDAC (0.0146 g, 0.076 mmol) and HOBt (0.0089 g, 0.066 mmol) in dichloromethane (3 μL) was stirred at RT for 5 minutes before N,N-diethylethylenediamine (0.0086 μL) was added. The resulting reaction mixture was stirred at RT overnight, washed with saturated aq. NaHCO3 solution (3×), brine, dried over MgSO4 and concentrated in vacuo. The crude was chromatographed over silica gel (CH₂Cl₂/MeOH/NH₃:90/9/1) to give the title compound as a colourless oil which crystallised upon standing (0.025 g, 0.051 mmol, 100%). ¹H NMR (300 MHz, CDCl₃): δ 1.06 (t, 6H), 2.58 (q, 4H), 2.66 (t, 2H), 3.36 (s, 3H), 3.54 (m, 2H), 3.97 (s, 3H), 6.36 (s, 1H), 6.91-7.32 (m, 11H), 8.29 (d, 1H), 8.35 (bs, 1H)

### Example 34

### N-(2-Dimethylamino-ethyl)-2,6-dimethoxy-3-nitro-benzamide

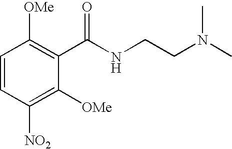

A flask was charged with 2,6-dimethoxy-3-nitrobenzoic acid (1 g, 4.4 mmol), 1-(3-dimethylaminopropyl)-3-ethyl-carbodiimide hydrochloride (1.27 g, 6.6 mmol), hydroxybenzotriazole (772 mg, 5.72 mmol) and N,N-dimethylethylene diamine (0.48 μL, 4.4 mmol). Dichloromethane (50 μL) was added and the suspension was stirred under air for 16 h. The now clear reaction mixture was washed consecutively with water (2×20 μL) and brine (1×20 μL). The organic solution was then briefly dried over sodium sulfate before being filtered and reduced in vacuo to give N-(N,N-dimethylaminoethylamine)-2,6-dimethoxy-3-nitrobenzamide. ¹H NMR (300 MHz, CDCl₃): δ 8.04-7.99 (2H, d), 6.77-6.72 (2H, d), 6.50-6.30 (1H, br s, NH), 3.97 (3H, s, MeO), 3.92 (3H, s, MeO), 3.60-3.45 (2H, m), 2.55-2.45 (2H, m), 2.25 (6H, s, Me₂N).

### Example 35

### 3-Amino-N-(2-dimethylamino-ethyl)-2,6-dimethoxy-benzamide

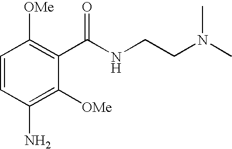

To a solution of N-(N,N-dimethylaminoethylamine)-2,6-dimethoxy-3-nitrobenzamide (1.31 g, 4.4 mmol) in dry methanol (50 mL) was added 10% palladium on carbon (50 mg). The reaction vessel was sealed and the atmosphere exchanged with nitrogen. The solution was then vigorously stirred and the atmosphere exchanged with hydrogen via a double balloon. Stirring continued for 16 h before the ballon was removed and the reaction mixture was filtered through a plug of celite (approx. 10 g). The residues were washed with excess methanol (approx. 100 μL) and the combined filtrates were reduced in vacuo returning a crude product which was chromatographed (Al₂O₃, dichloromethane/methanol/triethylamine, 90:9:1) to give N-(N,N-dimethylaminoethylamine)-3-amino-2,6-dimethoxybenzamide. ¹H NMR (300 MHz, CDCl₃): δ 7.99-7.91 (1H, m), 6.72-6.67 (1H, m), 3.88 (3H, s, MeO), 3.85 (3H, s, MeO), 3.72-3.67 (2H, m), 3.13-3.05 (2H, m), 2.72 (6H, s, Me₂N).

### Example 36

### N-(2-Dimethylamino-ethyl)-2,6-dimethoxy-3-(4-phenoxy-benzoylamino)-benzamide

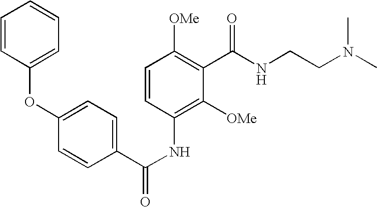

A flask was charged with N-(N,N-dimethylaminoethylamine)-3-amino-2,6-dimethoxybenzamide (8 mg, 32 μmol), hydroxybenzotriazole (5.6 mg, 46 μmol), N,N-dimethylaminopyridine (1 crystal) and 4-phenoxybenzoic acid (6.8 mg, 32 mmol).

Dichloromethane (10 μL) was added and the solution was stirred under air before PS-DCC (60 mg, approx. 64 μmol) was added. Stirring continued for 72 h before PS-trisamine (200 mg) was added and the resulting suspension stirred for 3 h. The resins were removed by filtration and further washed with dichloromethane (50 mL) and the combined organics were reduced in vacuo to give crude material which was chromatographed (Al₂O₃, dichloromethane/methanol/triethylamine, 90:9:1) to give the title product. ¹H NMR (300 MHz, CDCl₃): δ 8.45-8.38 (1H, d), 8.40-8.30 (1H, br s, NH), 7.60-7.30 (5H, m), 7.22-7.12 (2H, m), 7.08-7.00 (1H, d), 6.74-6.55 (1H, d), 6.52-6.48 (2H, m, Ar—H+NH), 3.89 (3H, MeO), 3.83 (3H, MeO), 3.58-3.52 (2H, m), 2.54-2.48 (2H, m), 2.26 (6H, s, Me₂N).

### Example 37

### N-(2-Dimethylamino-ethyl)-2,6-dimethoxy-3-(3-phenoxy-benzoylamino)-benzamide

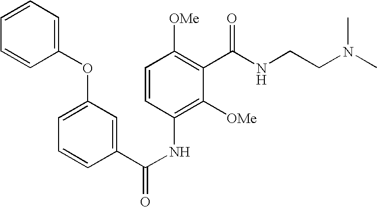

A flask was charged with N-(N,N-dimethylaminoethylamine)-3-amino-2,6-dimethoxybenzamide (80 mg, 0.32 mmol), hydroxybenzotriazole (43 mg, 0.32 mmol), N,N-dimethylaminopyridine (1 crystal) and 3-phenoxybenzoic acid (79 mg, 0.40 mmol). Dichloromethane (8 μL) was added and the solution was stirred under air before PS-DCC (350 mg, approx. 0.64 mmol) was added. Stirring continued for 72 h. Then PS-trisamine (100 mg) was added and stirred for 1 h before PS-iscocyanate (100 mg) was added and the resulting suspension stirred for a futher 1 h. The resins were removed by filtration and further washed with dichloromethane (50 μL) and the combined organics were reduced in vacuo to give crude material which was chromatographed (Al₂O₃, dichloromethane/methanol/triethylamine, 90:9:1) to give the title compound. ¹H NMR (300 MHz, CDCl₃): δ 8.42-8-38 (1H, d), 8.35-8.28 (1H, br s, NH), 7.48-7.35 (5H, m), 7.25-7.10 (2H, m), 7.08-7.10 (2H, d), 6.75-7.69 (1H, d), 6.68-6.48 (1H, br s, NH), 3.89 (3H, s, MeO), 3.83 (3H, s, MeO), 3.58-3.52 (2H, m), 2.53-2.49 (2H, m), 2.25 (6H, s, Me₂N).

### Example 38

### Biphenyl-4-carboxylic acid[3-(2-dimethylamino-ethylcarbamoyl)-2,4-dimethoxy-phenyl]-amide

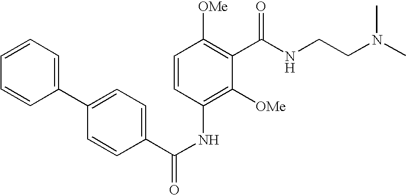

A flask was charged with N-(N,N-dimethylaminoethylamine)-3-amino-2,6-dimethoxybenzamide (80 mg, 0.32 mmol), hydroxybenzotriazole (43 mg, 0.32 mmol), N,N-dimethylaminopyridine (1 crystal) and biphenylacetic acid (79 mg, 0.40 mmol). Dichloromethane (8 μL) was added and the solution was stirred under air before PS-DCC (350 mg, approx. 0.64 mmol) was added. Stirring continued for 72 h. Then PS-trisamine (100 mg) was added and stirred for 1 h before PS-iscocyanate (100 mg) was added and the resulting suspension stirred for a futher 1 h. The resins were removed by filtration and further washed with dichloromethane (50 μL) and the combined organics were reduced in vacuo to give crude material which was chromatographed (Al₂O₃, dichloromethane/methanol/triethylamine, 90:9:1) to give the title compound. ¹H NMR (300 MHz, CDCl₃): δ 8.55-8.45 (1H, d), 8.45-8.35 (1H, br s, NH), 7.97-7.95 (2H, d), 7.80-7.70 (2H, d), 7.70-7.60 (2H, d), 7.60-7.40 (3H, m), 6.76-6.73 (1H, d), 6.60-6.50 (1H, br s, NH), 3.67 (3H, s, MeO), 3.86 (3H, s, MeO), 3.61-3.55 (2H, m), 256-2.52 (2H, m), 2.28 (6H, s, Me₂N); ¹³C NMR (75 MHz, CDCl₃) δ 165.4, 165.1, 153.6, 147.8, 140.3, 134.0, 129.4, 128.5, 127.9, 127.6, 125.5, 122.4, 120.4, 120.4, 107.3, 62.7, 58.0, 56.6, 46.3, 45.5, 37.7.

### Example 39

### 3-Bromo-5-[3-(4-bromo-phenoxy)-benzoylamino]-N-(2-dimethylamino-ethyl)-2,6-dimethoxy-benzamide

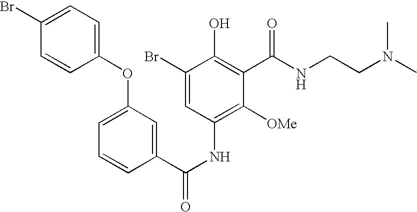

To a solution of Ex 37 (120 mg, 0.26 mmol) in dichloromethane (10 μL) with acetic acid (1 drop) was added bromine (27 μL, 0.52 mmol) dropwise. The brown solution was then stirred for 16 h before a saturated solution of sodium thiosulfate (10 μL) was added and shaken to remove excess bromine. The organic solution was further washed with water (10 μL) and brine (10 μL) before being dried over sodium sulphate, filtered and reduced in vacuo. The crude material was chromatographed (Al₂O₃, dichloromethane/methanol/triethylamine, 90:9:1) to give the title compound. ¹H NMR (300 MHz, CDCl₃): δ 9.10-9.00 (1H, br app s, NH), 8.88 (1H, s), 8.60-8.50 (1H, br s, NH), 7.64-7.60 (1H, dt), 7.57-7.56 (1H, t), 7.53-7.45 (3H, m), 7.21-7.18 (1H, dd), 6.98-6.93 (2H, d), 3.89 (3H, s, MeO), 3.58-3.53 (2H, q, CH₂NH), 2.59-2.57 (2H, t, CH₂N), 2.32 (6H, s, Me₂N).

### Example 40

### Biphenyl-4-carboxylic acid[5-bromo-3-(2-dimethylamino-ethylcarbamoyl)-2,4-dimethoxy-phenyl]-amide

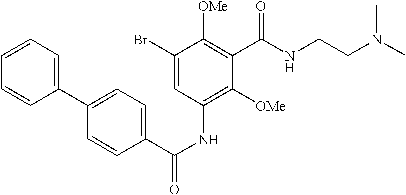

To a solution of Ex 38 (120 mg, 0.26 mmol) in dichloromethane (10 μL) with acetic acid (1 drop) was added bromine (27 μL, 0.52 mmol) dropwise. The brown solution was then stirred for 16 h before a saturated solution of sodium thiosulfate (10 μL) was added and shaken to remove excess bromine. The organic solution was further washed with water (10 μL) and brine (10 μL) before being dried over sodium sulphate, filtered and reduced in vacuo. The crude material was chromatographed (Al₂O₃, dichloromethane/methanol/triethylamine, 90:9:1) to give the title compound. ¹H NMR (300 MHz, CDCl₃): δ 8.82 (1H, s), 8.50-8.60 (1H, br s, NH), 7.98-7.93 (2H, d), 7.77-7.72 (2H, d), 7.67-7.62 (2H, m), 7.58-7.35 (3H, m), 6.92-6.80 (1H, br s, NH), 3.96 (3H, s, MeO), 3.89 (3H, s, MeO), 3.64-3.59 (2H, q, CH₂NH), 2.65-2.58 (2H, t, CH₂N), 2.32 (6H, s, Me₂N).

### Example 41

### Biphenyl-4-carboxylic acid[5-bromo-3-(2-dimethylamino-ethylcarbamoyl)-4-hydroxy-2-methoxy-phenyl]-amide

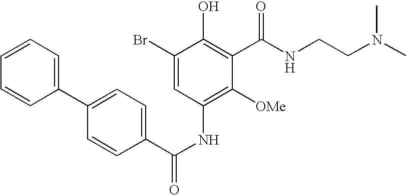

From the above reaction a second product was isolated and identified as Ex 41.

¹H NMR (300 MHz, CDCl₃): δ 9.07-9.04 (1H, br s, NH), 8.96 (1H, s), 8.75-8.65 (1H, br s, NH), 8.02-7.99 (2H, d), 7.76-7.73 (2H, d), 7.68-7.65 (2H, d), 7.60-7.35 (3H, m), 3.90 (3H, s, MeO), 3.59-3.54 (2H, q, CH₂NH), 2.58-2.54 (2H, t, CH₂N), 2.32 (6H, s, Me₂N).

### Example 42

### 1-Bromo-2,4-dimethoxy-3-methyl-benzene

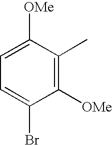

To a solution of 2,6-dimethoxytoluene (5 g, 33 mmol) in dichloromethane (100 μL) and acetic acid (1 drop) held at 0° C. was added bromine (1.67 μL, 33 mmol) dropwise. The pale brown solution was stirred for a further 5 h before being washed with a saturated solution of sodium thiosulfate (20 μL), sodium bicarbonate (20 μL), water (20 μL) and brine (20 μL). The organic solution was then dried over sodium thiosulfate, filtered and evaporated in vacuo to give the title compound. ¹H NMR (300 MHz, CDCl₃) δ 7.35-7.32 (1H, d), 6.56-6.53 (1H, d), 3.82 (3H, s, MeO), 3.81 (3H, s, MeO), 2.22 (3H, s, CH₃).

### Example 43

### 3,5-Dimethoxy-4-methyl-phenylamine

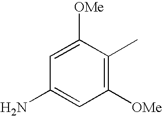

To a freshly prepared suspension of potassium amide (from potassium 12.87 g, 330 mmol) in liquid ammonia (300 μL) held at −78° C. was added example 9 (7.6 g, 33 mmol) dropwise over twenty minutes. The resulting suspension was stirred for a further 3 h and then excess potassium amide was quenched carefully with solid ammonium chloride (10 g) added portionwise over thirty minutes. Toluene (200 μL) was added and the liquid ammonia allowed to evaporate. The organic solution was then washed with water (3×100 μL) before being shaken with hydrochloric acid (6 N, 200 μL). The nascent precipitate was then collected by filtration and further washed with water (100 μL). The residue was stirred with sodium hydroxide (10 N, 100 μL) for 1 h to form the free aniline, which was collected by filtration. The residues were washed with water (3×20 μL) and dried in vacuo to give the title compound.

### Example 44

### N-(3,5-Dimethoxy-4-methyl-phenyl)-4-phenoxy-benzamide

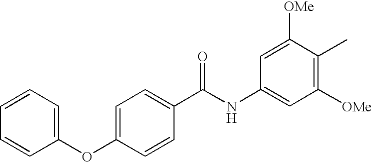

A flask was charged with Ex 43 (334 mg, 2 mmol), 4-phenoxybenzoic acid (471 mg, 2.2 mmol), 1-(3-dimethylaminopropyl)-3-ethyl-carbodiimide hydrochloride (573 mg, 3 mmol) and hydroxybenzotriazole (351 mg, 2.6 mmol). Dichloromethane (20 μL) was added and the suspension was stirred for 100 h. The now clear solution was washed with hydrochloric acid (1 N, 20 μL), sodium bicarbonate (20) and water (20 μL). The organic solution was dried over sodium sulphate, filtered and reduced in vacuo. The crude material was chromatographed (Al₂O₃, dichloromethane/methanol/triethylamine, 98:1:1) to give the title compound. ¹H NMR (300 MHz, CDCl₃): δ 7.88-7.85 (2H, d), 7.75 (1H, s), 7.50-7.40 (2H, m), 7.25-7.15 (1H, m), 7.12-7.05 (4H, m), 6.93 (2H, s), 5.95 (1H, s), 3.85 (6H, s, MeO), 2.09 (3H, s, CH₃).

### Example 45

### 2-Methoxy-4-[3-(4-trifluoromethyl-phenyl)-ureido]-benzoic acid

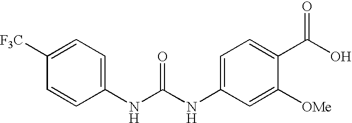

To a solution of 4-amino-2-methoxybenzoic acid (3.4 g, 20.3 mmol) in dry dichloromethane (300 mL) under inert atmosphere was 4-trifluoromethylphenyl isocyanate (5.0 g, 26.7 mmol) added drop wise. The reaction was stirred over night at room temperature and a precipitate was formed during the reaction. The precipitate was filtered and washed with dichloromethane and gave 5.7 g (79%) of the title product. ¹H NMR (300 MHz, dmso-d₆): δ 3.8 (s, 3H), 6.9 (dd, 1H), 7.4 (d, 1H), 7.4-7.7 (m, 5H), 9.2 (d, 2H), 12.2 (s, 1H). LCMS(an20n15); RT=8.306 min 352.9 m/z

### Example 46

### 4-[3-(4-Trifluoromethoxy-phenyl)-ureido]-benzoic acid

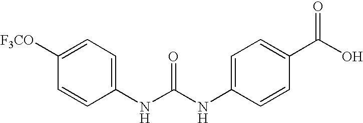

Using the same procedure as described above was the title product synthesised from 4-amino-benzoic acid (1.2 g, 7.6 mmol) and 4-trifluoromethoxyphenyl isocyanate (2.0 g, 9.8 mmol) giving 2.7 g (quant.) of the product.

LCMS(an20n15); RT=7.503 min, 338.8 m/z.

### Example 47

### N-(2-Diethylamino-ethyl)-4-[3-(4-trifluoromethoxy-phenyl)-ureido]-benzamide

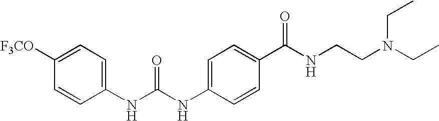

To a solution of procainamide (26 mg, 0.112 mmol) in dichloromethane (1.5 mL) under inert atmosphere were triethylamine (31 μL) and 4-trifluoromethoxyphenyl isocyanate (30 μL, 0.145 mmol) added. The reaction was stirred for three days. PS-Trisamine (0.16 g, 3.58 mmol/g, 0.56 mmol) was added and the reaction was stirrede for two more days. The resin was filtered off and the reaction mixture was concentrated in vacuo. The crude product was purified by acidic ion exchange chromatography (SCX-colon) giving 29 mg (59%) of the title product. LCMS (an20p10): RT=5.52 min, (M−1)=439.0 m/z.

### Example 48

### N-(2-Diethylamino-ethyl)-2-methoxy-4-[3-(4-trifluoromethoxy-phenyl)-ureido]-benzamide

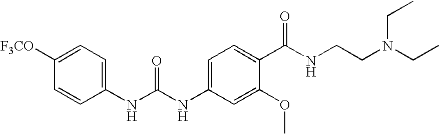

To a solution of 2-methoxy-4-[3-(4-trifluoromethoxy-phenyl)-ureido]-benzoic acid (example 153) (50 mg, 0.135 mmol) in dichloromethane (3.5 mL) and dimethylformamide (0.35 mL) was added to polystyrene-DCC (0.5 g, 1.27 mmol/g, 0.64 mmol). Thereafter were HOBT (40 mg, 0.30 mol) and N¹,N¹-diethyl-ethyldiamine (18 μL, 16.5 mg 0.14 mmol) added and the reaction was stirred over night. The resin was filtered off and rinsed with dichloromethane. The reaction mixture was concentrated in vacuo. The crude product was purified with acidic ion exchange chromatography (SCX-colon) giving 12 mg (18%) of the title product. LCMS (an20p10): RT=4.95 min, (M+1)=469.0 m/z.

### Example 49

### 4-Amino-N-[2-(4-benzyl-piperazin-1-yl)-ethyl]-2-methoxy-benzamide

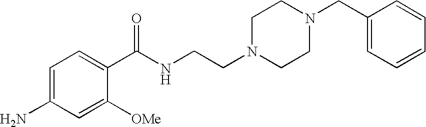

To a solution of 2-(4-Benzyl-piperazin-1-yl)-ethylamine (10 g, 45 mmol) in dry dichloromethane (500 mL) were EDC (11.3 g, 58 mmol), 2-methoxy-4-nitro-benzoic acid (11 g, 55 mmol) and HOBt (7.6 g, 56 mmol) added. The reaction mixture was left stirring at room temperature for four days. To the reaction was dichloromethane (3 L) added which was washed with Na2CO3 (sat.) (0.5 L). and the water phase was extracted with dichloromethane (3 L). The combined organic phases were dried (MgSO4), and concentrated in vacuo. The crude product (30 g) was chromatographed (silica, EtOAc/Heptane/triethylamine, 60:33:7) giving 17 g of 4-nitro-N-[2-(4-benzyl-piperazin-1-yl)-ethyl]-2-methoxy-benzamide (96%). The product (4.3 g, 10.7 mmol) was dissolved in methanol (430 mL) and 5% Pt/C (430 mg) was added under a nitrogen flow. The mixture was stirred in an H₂ atmosphere over night. The catalyst was filtered off using a pad of celite and the remaining solution was concentrated in vacuo giving 3.5 g (89%) of the title product. LCMS (an20p15); RT=2.73 min, (M+1)=369.

According to the procedure outlined in example 48 were the following compounds prepared utilizing Ex 138 and the corresponding primary amines to the R-group, if not noted otherwise;

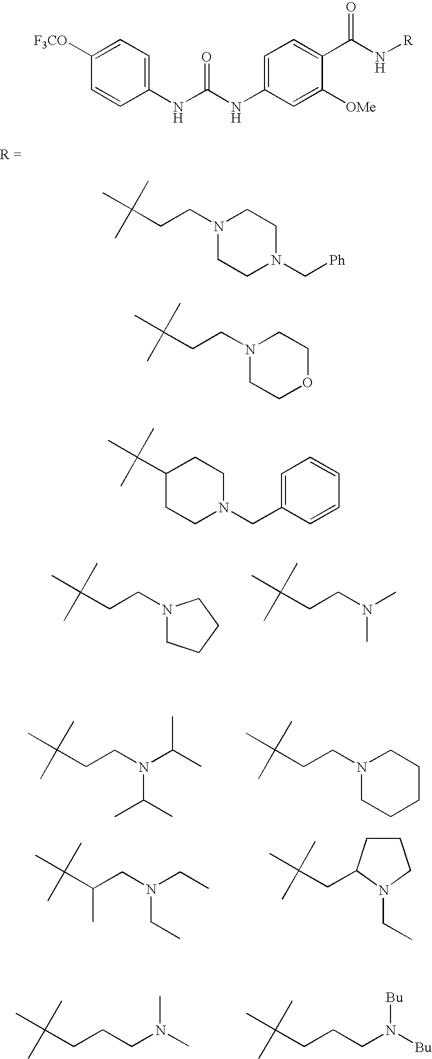

### Example 50

### N-[2-(4-Benzyl-piperazin-1-yl)-ethyl]-2-methoxy-4-[3-(4-trifluoromethoxy-phenyl)-ureido]-benzamide

To a solution of 4-Amino-N-[2-(4-benzyl-piperazin-1-yl)-ethyl]-2-methoxy-benzamide (example 49) (60 mg, 0.163 mmol) in dichloromethane (2 mL) was 4-trifluoromethoxyphenyl isocyanate (45 mg, 0.22 mmol) added and the reaction was stirred under inert atmosphere over weekend. PS-Trisamine (100 mg, 3.58 mol/g) was added and the reaction mixture was continuously stirred over night. The resin was filtered off and washed twice with dichloromethane. The solvent was removed in vacuo. The crude product was purified through acidic ion exchange chromatography (SCX-colon) and as eluent was dichloromethane followed with methanol used. From the methanol was isolated 27 mg of the title product. LCMS(an20p15); RT=6.61 min, (M+1)=572.1

### Example 51

### 2-Methoxy-N-(2-morpholin-4-yl-ethyl)-4-[3-(4-trifluoromethoxy-phenyl)-ureido]-benzamide

Ex 138 and 3-Morpholin-4-yl-ethylamine were coupled giving 11 mg (16%) of the title product. LCMS(an20p15); RT=5.99 min, (M+1)=483.0.

### Example 52

### N-(1-Benzyl-piperidin-4-yl)-2-methoxy-4-[3-(4-trifluoromethoxy-phenyl)-ureido]-benzamide

Ex 138 and 1-Benzyl-piperidin-4-ylamine were coupled giving 11 mg (46%) of the title product. LCMS(an20p15); RT=5.90 min, (M+1)=543.0.

### Example 53

### 2-Methoxy-N-(2-pyrrolidin-1-yl-ethyl)-4-[3-(4-trifluoromethoxy-phenyl)-ureido]-benzamide

Ex 138 and 2-Pyrrolidin-1-yl-ethylamine were coupled giving 12 mg (19%) of the title product. LCMS(an20p15); RT=8.22 min, (M+1)=467.0

### Example 54

### N-(2-Dimethylamino-ethyl)-2-methoxy-4-[3-(4-trifluoromethoxy-phenyl)-ureido]-benzamide

Ex 138 and N¹,N¹-Dimethyl-ethane-1,2-diamine were coupled giving 7.8 mg (13%) of the title product. LCMS(an20p15); RT=5.91 min, (M+1)=440.9

### Example 55

### N-(2-Diisopropylamino-ethyl)-2-methoxy-4-[3-(4-trifluoromethoxy-phenyl)-ureido]-benzamide

Ex 138 and N¹,N¹-Diisopropyl-ethane-1,2-diamine were coupled giving 17 mg (26%) of the title product. LCMS(an20p15); RT=7.92 min, (M+1)=497.0

### Example 56

### 2-Methoxy-N-(2-piperidin-1-yl-ethyl)-4-[3-(4-trifluoromethoxy-phenyl)-ureido]-benzamide

Ex 138 and 2-Piperidin-1-yl-ethylamine N¹,N¹-Diethyl-propane-1,2-diamine were coupled giving 12 mg (18%) of the title product. LCMS(an20p15); RT=8.53 min, (M+1)=481.0

### Example 57

### N-(2-Diethylamino-1-methyl-ethyl)-2-methoxy-4-[3-(4-trifluoromethoxy-phenyl)-ureido]-benzamide

Ex 138 and N¹,N¹-Diethyl-propane-1,2-diamine were coupled giving 9.2 mg (14%) of the title product. LCMS(an20p15); RT=6.17 min, (M+1)=483.0

### Example 58

### N-(1-Ethyl-pyrrolidin-2-ylmethyl)-2-methoxy-4-[3-(4-trifluoromethoxy-phenyl)-ureido]-benzamide

Ex 138 and C-(1-Ethyl-pyrrolidin-2-yl)-methylamine were coupled giving 12 mg (19%) of the title product. LCMS(an20p15); RT=7.52 min, (M+1)=481.0

### Example 59

### N-(3-Dimethylamino-propyl)-2-methoxy-4-[3-(4-trifluoromethoxy-phenyl)-ureido]-benzamide

Ex 138 and N¹,N¹-Dimethyl-propane-1,3-diamine were coupled giving 12 mg (20%) of the title product. LCMS(an20p15); RT=5.93 min, (M+1)=455.0

### Example 60

### N-(3-Dibutylamino-propyl)-2-methoxy-4-[3-(4-trifluoromethoxy-phenyl)-ureido]-benzamide

Ex 138 and N¹,N¹-Dibutyl-propane-1,3-diamine were coupled giving 14 mg (20%) of the title product. LCMS(an20p15); RT=7.25 min, (M+1)=539.1

### Example 61

### 4-Amino-2-methoxy-N-(3-morpholin-4-yl-propyl)-benzamide

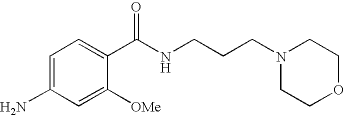

2-methoxy-4-nitro-benzoic acid (3.0 g, 15.5 mmol) was dissolved in THF(180 ml) and the mixture was heated to reflux (70° C.). Carbonyl diimidazol (3.7 g, 22.8 mmol) was added in 3 portions with 20 minutes intervals—with continued refluxing. After the last addition reaction is allowed to reflux for another 1 h. The reaction mixture was cooled to room temperature followed by addition of 3-morpholin-4-yl-propylamine (4.4 g, 30.4 mmol) and the reaction was left overnight. The solvent was removed in vacuo and to the crude product was added a mixture of 200 ml EtOAc and 200 ml of water. The organic phase is washed with 2*200 ml water and 1*200 ml of brine. The combined organic phases was dried over MgSO₄ and concentrated giving a clear oil. Crystallisation can be obtained by adding diethylether followed by evaporation. The product (7.6 g, 23 mmol) was dissolved in methanol (120 ml) and 10% Pd/C (40 mg) was added. A pressure of hydrogen atmosphere was applied and the reaction was left over night. Filtration through a plug of celite gave 7.36 g of the title product (94% over all yield).

¹H-NMR (300 MHz, CD₃Cl): δ 1H, 7.79 (s, 1H), 4.03 (s, 2H), 3.90 (s, 3H).

### Example 62

### 2-Methoxy-N-(3-morpholin-4-yl-propyl)-4-[3-(4-trifluoromethoxy-phenyl)-ureido]-benzamide

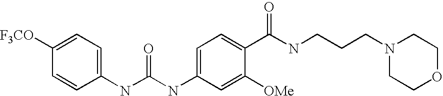

To a solution of 4-Amino-2-methoxy-N-(3-morpholin-4-yl-propyl)-benzamide (20 mg, 0.068 mmol) (example 61) in dichloromethane (1 mL) was 4-trifluoromethoxyphenyl isocyanate (19 □L, 0.136 mmol) added and the reaction was stirred under inert atmosphere for three days. The solvent was removed in vacuo. The crude product was chromatographed (silica, CH₂Cl₂/methanol, 92:8) giving 3.4 mg of the title product. ¹H NMR (300 MHz, CD₃Cl): δ 3.89 (s, 3H), 8.04 (br t, 1H), 9.09 (s, 1H), 9.12 (s, 1H). LCMS(an10p15): found (M+1)=497.

### Example 63

### 4-Amino-N-(2-diethylamino-ethyl)-2-methoxy-benzamide

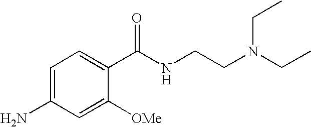

A solution of 2-Methoxy-4-nitrobenzoic acid (1.5 g, 7.6 mmol) in dry dichloromethane (15 mL) was placed on an ice bath whereupon oxalyl chloride (0.6 mL, 6.8 mmol) followed by N,N-dimethylformamide (2 μL) were added under inert atmosphere. The mixture was stirred for 30 min at 0° C. followed by 1 h in room temperature. Triethylamine (2.1 mL, 15 mmol) and N,N-diethylethylenediamine (1.1 mL, 7.6 mmol) were added and a precipitation was formed. The reaction mixture was stirred for 48 h. To the reaction mixture was added EtOAc (60 mL) and the organic layer was washed with Na₂CO₃ (sat.), dried (MgSO₄), and concentrated in vacuo. The reaction was giving 2.0 g of N-(2-diethylamino-ethyl)-2-methoxy-4-nitro-benzamide (99%). ¹H NMR (300 MHz, CD₃Cl): δ 1.06 (t, 6H), 4.07 (s, 3H).

The product (2.0 g, 6.8 mmol) was dissolved in ethanol (20 mL) and 10% Pd/C (50 mg) was added and thereafter was the flask evacuated and filled with nitrogen. The mixture was stirred in an H₂ atmosphere 48 h. The catalyst was filtered off using a pad of celite and the remaining solution was concentrated in vacuo. The crude product was chromatographed (silica, dichloromethane/ethanol/ammoniak, 100:15:1.5) giving 1.0 g (57%) of the title product. ¹H NMR (300 MHz, CD₃Cl): δ 1.05 (t, 6H), 2.53-2.65 (m, 6H).

### Example 64

### N-(2-Diethylamino-ethyl)-2-methoxy-4-[3-(4-trifluoromethyl-phenyl)-ureido]-benzamide

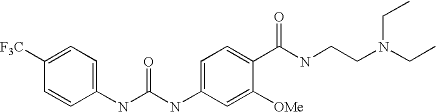

To a solution of N-(2-Diethylamino-ethyl)-2-methoxy-4-amino-benzamide (20 mg, 0.075 mmol) (example 74) in dichloromethane (1 mL) was trifluorophenylisocyanate (28 mg, 0.15 mmol) added and the reaction was stirred under inert atmosphere for four days. A white precipitate had been formed. The solvent was removed in vacuo. The crude product was chromatographed (silica, CH₂Cl₂/methanol/ammoniak, 200:10:1) giving 11 mg of the title product. ¹H NMR (300 MHz, CD₃Cl): δ 1.05 (t, 6H), 2.56 (q, 4H), 2.65 (t, 2H), 3.54 (q, 2H), 3.94 (s,3H), 6.48 (dd, 1H), 7.56 (q, 4H), 7.83 (d, 1H), 7.92 (d, 1H), 8.74 (t, 1H), 8.91 (s, 1H), 8.93 (s, 1H).

### Example 65

### 4-Amino-N-(1-benzyl-piperidin-4-yl)-2-methoxy-benzamide

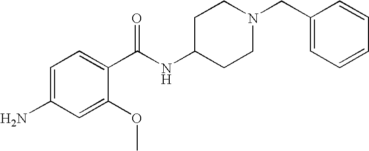

The title compound was prepared according to the example described in example 74 giving after the two reaction steps and the purification procedure 0.6 g (33%) of the product. ¹H NMR (300 MHz, CD₃Cl): δ 3.52 (s, 2H), 4.02-4.11 (m, 1H), 3.86 (s, 3H).

A general method for preparing unsymmetrical amines:

### Example 66

### N¹-methyl,N¹-ethyl-ethyldiamine hydro chloride

To a suspension of bromoethylphtalimide (8.9 g, 35 mmol) in dry xylene (18 mL) was N-ethylmethylamine (6.25 mL, 73 mmol) added and the reaction was stirred over night at 150° C. The reaction was allowed to reach room temperature before it was made basic with 2 M Na₂CO₃-solution (pH=9). Thereafter was the reaction extracted with EtOAc (3×70 mL) and the combined organic phases dried (MgSO₄) and evaporated giving a brownish oil. Water (2 mL) and 12 N HCl (12 mL) were added and the solution was heated for 6 h at 130° C. when a precipitation was formed. The precipitate was filtered off and washed with cold water and the water phase was evaporated giving 4.8 g (78%) of the title product.

According to the General Procedures Described hereby, the Following Compounds were Prepared:

Method A: To a solution of Ex. 45 (30 mg, 0.085 mmol) in dichloromethane (0.25 mL) and dimethylformamide (0.25 mL) was HOBT (12 mg, 0.085 mmol) added and the solution was cooled to 0° C. whereupon EDAC (16 mg, 0.085 mmol) was added. The reaction was left at 0° C. for 20 min before the amine (1-1.5 equiv.) and diisopropylethylamine (1-3 equiv.) were added and the stirring continued at room temperature for one day or more. EtOAc was added to the reaction mixture and the organic phase was washed with NaHCO₃ (sat). The aqueous phase was extracted with EtOAc and the combined organic phases was dried (MgSO₄) and concentrated giving the crude product.

Method B: To a solution of Ex. 45 (70 mg, 0.20 mmol) in dichloromethane (3.5 mL) and N,N-dimethylformamide (0.35 mL) were PS-DDC (0.5 g, 1.27 mmol/g), HOBT (40 mg, 0.29 mmol) and the amine 1 equiv.) added and the mixture was stirred over night. The reaction mixture was filtered off and washed with dichloromethane, and concentrated in vacuo. The crude product was purified by acidic ion exchange chromatography (SCX-colon) the product.

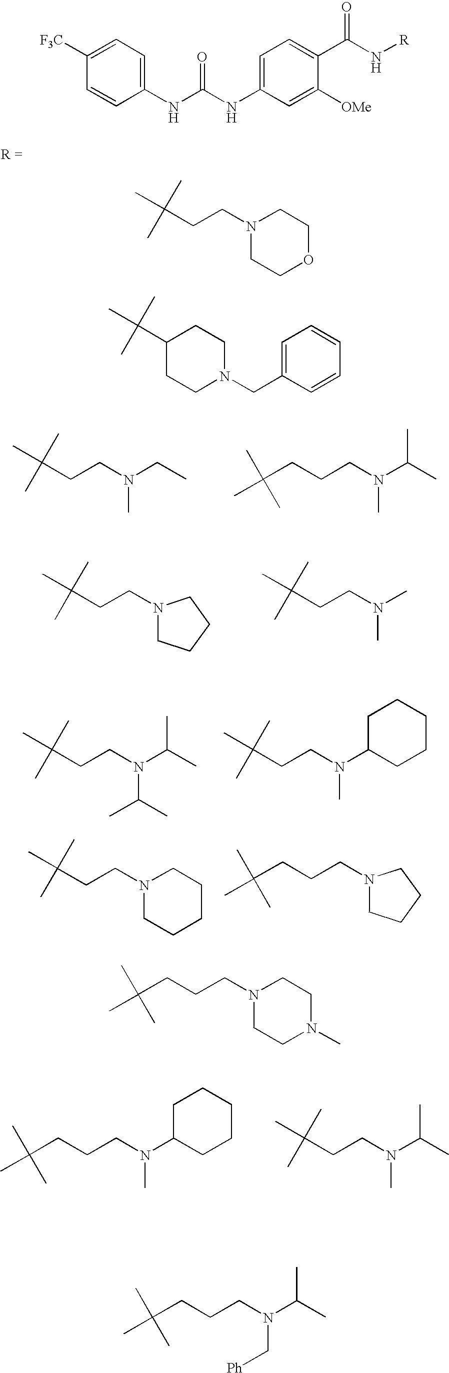

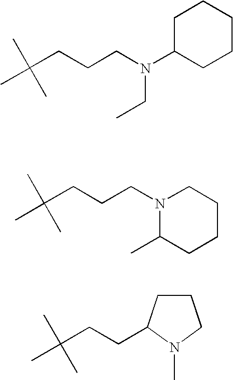

### Example 67

### 2-Methoxy-N-(2-morpholin-4-yl-ethyl)-4-[3-(4-trifluoromethyl-phenyl)-ureido]-benzamide

According to method B was the title compound synthesised giving 35 mg (53%) of the product. LCMS (an20p15): RT=5.72 min, (M+1)=467.

### Example 68

### N-(1-Benzyl-piperidin-4-yl)-2-methoxy-4-[3-(4-trifluoromethyl-phenyl)-ureido]-benzamide

According to method A was the title compound synthesised giving the product. LCMS (an20p15): RT=6.49 min (M+1)-527. ¹H NMR (300 MHz, CD₃Cl): δ 3.48 (s, 2H), 3.93 (s, 3H), 3.93-4.05 (m, 1H), 8.12 (s, 1H), 8.15 (s, 1H).

### Example 69

### N-[2-(Ethyl-methyl-amino)-ethyl]-2-methoxy-4-[3-(4-trifluoromethyl-phenyl)-ureido]-benzamide

According to method A was the title compound synthesised giving the product. LCMS (an20p15): RT=6.49 min, (M+1)=439. ¹H NMR (300 MHz, CD₃Cl): δ 1.08 (t, H), 2.27 (s, 3H), 3.95 (s, 3H), 9.01 (s, 1H), 9.03 (1H).

### Example 70

### N-[3-(Isopropyl-methyl-amino)-propyl]-2-methoxy-4-[3-(4-trifluoromethyl-phenyl)-ureido]-benzamide

According to method A was the title compound synthesised giving the product. ¹H NMR (300 MHz, CD₃Cl): δ 0.98 (d, 6H), 2.20 (s, 3H), 3.91 (s, 3H), 8.66 (s, 1H), 8.90 (s, 1H).

### Example 71

### 2-Methoxy-N-(2-pyrrolidin-1-yl-ethyl)-4-[3-(4-trifluoromethyl-phenyl)-ureido]-benzamide

According to method B was the title compound synthesised giving 29 mg (46%) of the product. LCMS (an20p15): RT=5.79 min, (M+1)=451.

### Example 72

### N-(2-Dimethylamino-ethyl)-2-methoxy-4-[3-(4-trifluoromethyl-phenyl)-ureido]-benzamide

According to method B was the title compound synthesised giving 13 mg (23%) of the product. LCMS (an20p15): RT=5.66 min, (M+1)=425.

### Example 73

### N-(2-Diisopropylamino-ethyl)-2-methoxy-4-[3-(4-trifluoromethyl-phenyl)-ureido]-benzamide

According to method B was the title compound synthesised giving 18 mg (27%) of the product. LCMS (an20p15): RT=6.32 min, (M+1)=481.0 m/z.

### Example 74

### N-[2-(Cyclohexyl-methyl-amino)-ethyl]-2-methoxy-4-[3-(4-trifluoromethyl-phenyl)-ureido]-benzamide

According to method A was the title compound synthesised giving the product. LCMS (an20p15): RT=6.65 min, (M+1)=493. ¹H NMR (300 MHz, CD₃Cl): δ 1.63 (d, 1H), 2.31 (s, 3H), 3.91 (s, 3H), 9.08 (s, 2H).

### Example 75

### 2-Methoxy-N-(2-piperidin-1-yl-ethyl)-4-[3-(4-trifluoromethyl-phenyl)-ureido]-benzamide

According to method B was the title compound synthesised giving 14 mg (22%) of the product. LCMS (an20p15): RT=5.99 min, (M+1)=465.

### Example 76

### 2-Methoxy-N-(3-pyrrolidin-1-yl-propyl)-4-[3-(4-trifluoromethyl-phenyl)-ureido]-benzamide

According to method A was the title compound synthesised giving the product. LCMS (an20p15): RT=5.93 min, (M+1)=465. ¹H NMR (300 MHz, CD₃Cl): δ 3.90 (s, 3H), 9.08 (s, 1H), 9.14 (s, 1H).

### Example 77

### 2-Methoxy-N-[3-(4-methyl-piperazin-1-yl)-propyl]-4-[3-(4-trifluoromethyl-phenyl)-ureido]-benzamide

According to method A was the title compound synthesised giving the product. LCMS (an20p15): RT=5.05 min, (M+1)=494. ¹H NMR (300 MHz, CD₃Cl): δ 2.28 (s, 3H), 3.94 (s, 3H), 9.03 (s, 1H), 9.07 (s, 1H).

### Example 78

### N-[3-(Cyclohexyl-methyl-amino)-propyl]-2-methoxy-4-[3-(4-trifluoromethyl-phenyl)-ureido]-benzamide

According to method A was the title compound synthesised giving the product. ¹H NMR (300 MHz, CD₃Cl): δ 1.61 (d, 1H), 2.26 (s, 3H), 3.90 (s, 3H), 9.07 (s, 1H), 9.13 (s, 1H).

### Example 79

### N-[2-(Isopropyl-methyl-amino)-ethyl]-2-methoxy-4-[3-(4-trifluoromethyl-phenyl)-ureido]-benzamide

According to method A was the title compound synthesised giving the product. LCMS (an20p15): RT=6.12 min, (M+1)=453. ¹H NMR (300 MHz, CD₃Cl): δ 1.05 (d, 6H), 2.27 (s, 3H), 2.89-2.98 (m, 1H), 3.92 (s, H), 9.07 (s, 1H).

### Example 80

### N-[3-(Benzyl-isopropyl-amino)-propyl]-2-methoxy-4-[3-(4-trifluoromethyl-phenyl)-ureido]-benzamide

According to method A was the title compound synthesised giving the product. LCMS (an20p15): RT=6.27 min, (M+1)=543. ¹H NMR (300 MHz, CD₃Cl): δ 1.01 (d, 6H), 3.55 (s, 2H), 3.82 (s, 3H), 9.09 (s, 1H), 9.15 (s, 1H).

### Example 81

### N-[3-(Cyclohexyl-ethyl-amino)-propyl]-2-methoxy-4-[3-(4-trifluoromethyl-phenyl)-ureido]-benzamide

According to method A was the title compound synthesised giving the product. LCMS (an20p15): RT=6.13 min, (M+1)=521. ¹H NMR (300 MHz, CD₃Cl): δ 1.00 (t, 3H), 1.61 (d, 1H), 3.89 (s, 3H), 9.10 (s, 1H), 9.16 (s, 1H).

### Example 82

### 2-Methoxy-N-[3-(2-methyl-piperidin-1-yl)-propyl]-4-[3-(4-trifluoromethyl-phenyl)-ureido]-benzamide

According to method A was the title compound synthesised giving the product. LCMS (an20p15): RT=6.26 min, (M+1)=493. ¹H NMR (300 MHz, CD₃Cl): δ 1.03 (d, 3H), 3.92 (s, 3H), 9.03 (s, 1H), 9.09 (s, 1H).

### Example 83

### 2-Methoxy-N-[2-(1-methyl-pyrrolidin-2-yl)-ethyl]-4-[3-(4-trifluoromethyl-phenyl)-ureido]-benzamide

According to method A was the title compound synthesised giving the product. LCMS (an20p15): RT=5.76 min, (M+1)=465. ¹H NMR (300 MHz, CD₃Cl): δ 2.30 (s, 3H), 3.94 (s, 3H), 9.00 (s, 1H), 9.02 (s, 1H).

### Example 84

### N-(2-Diethylamino-ethyl)-2-methoxy-4-[3-(3-trifluoromethoxy-phenyl)-ureido]-benzamide

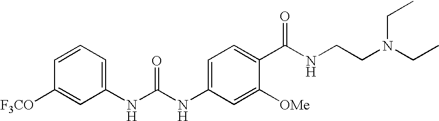

### Example 85

### N-(2-Diethylamino-1-methyl-ethyl)-2-methoxy-4-[3-(4-trifluoromethoxy-phenyl)-ureido]-benzamide

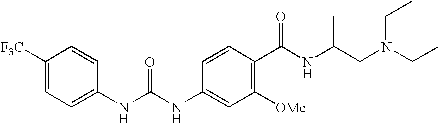

To a solution of 4-amino-N-(2-diethylamino-1-methyl-ethyl)-2-methoxy-benzamide (56 mg, 0.2 mmol) (synthesised using the same method as for example 74) in dry dichloromethane (5 mL) was 4-trifluorotosyl isocyanate (35 □L, 0.25 mmol) added and the reaction was stirred under inert atmosphere for four days. The solvent was removed in vacuo. The crude product was chromatographed (Al₂O₃, CH₂Cl₂/methanol/ammoniak, 10:0.25+0.5%) giving 27 mg of the title product (29%).

### Example 86

### 4-Amino-2-methoxy-N-(3-piperidin-1-yl-propyl)-benzamide

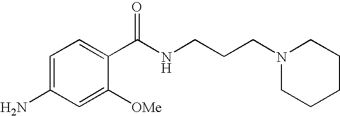

To a refluxing solution of 2-methoxy-4-nitro-benzoic acid (5.6 g, 29 mmol) in dry THF (mL) was carbonyldiimidazol (3×2.3 g, 42 mmol) added in three portions with 15 min in between. After 20 minutes continuous refluxing the reaction was cooled to room temperature and 3-amino-propyl-piperidine (4.5 g, 32 mmol) was added. The reaction mixture was left stirring over night. Water and EtOAc was added and the organic phase was separated, dried (Na₂SO₄), and concentrated in vacuo. The crude product was chromatographed (silica, CH₂Cl₂/methanol/ammoniak, 9:1+1%) giving 6.8 g of 2-methoxy-4-nitro-N-(3-piperidin-1-yl-propyl)benzamide (74%). The product was dissolved in ethanol (250 mL) and 10% Pd/C (200 mg) was added under a nitrogen flow. A balloon containing H₂ was collected to the flask and the reaction mixture was stirred for 2 h. The catalyst was filtered off using a pad of celite and the remaining solution was concentrated in vacuo giving 4.9 g (80%) of the title product.

### Example 87

### 2-Methoxy-N-(3-piperidin-1-yl-propyl)-4-[3-(4-trifluoromethoxy-phenyl)-ureido]-benzamide

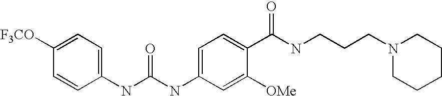

To a solution of example 97 (58 mg, 0.2 mmol) in dichloromethane was 4-trifluoromethoxyphenyl isocyanate (35 μL, 0.25 mmol) added and the reaction was stirred over night in inert atmosphere. The solvent was removed in vacuo. The crude product was chromatographed (Al₂O₃, CH₂Cl₂/methanol/ammoniak, 10:0.25+0.5%) giving 20 mg of the title product (20%).

According to the procedure described in example 98 (from anilines and isocyanates) or according to the general method described below (from anilines and carboxylic acid) were the following compounds prepared:

General Method for Preparing Ureas from Anilines and Carboxylic Acids:

To a solution of the carboxylic acid (0.25 mmol) in dry toluene (5 mL) under inert atmosphere were diphenylphosphoryl azide (54 μL, 0.25 mmol) and triethylamine (35 μL, 0.25 mmol) and the reaction mixture was heated to reflux for 1 h. 4-Amino-2-methoxy-N-(3-piperidin-1-yl-propyl)-benzamide, Example 97 (44 mg, 0.15 mmol) dissolved in hot toluene (2 mL) was added and the reaction mixture was left over night in room temperature. The solvent was removed in vacuo. The crude product was usually purified through chromatography (silica, CH₂Cl₂/methanol/ammoniak, 10:0.25+0.5%) giving the desired product.

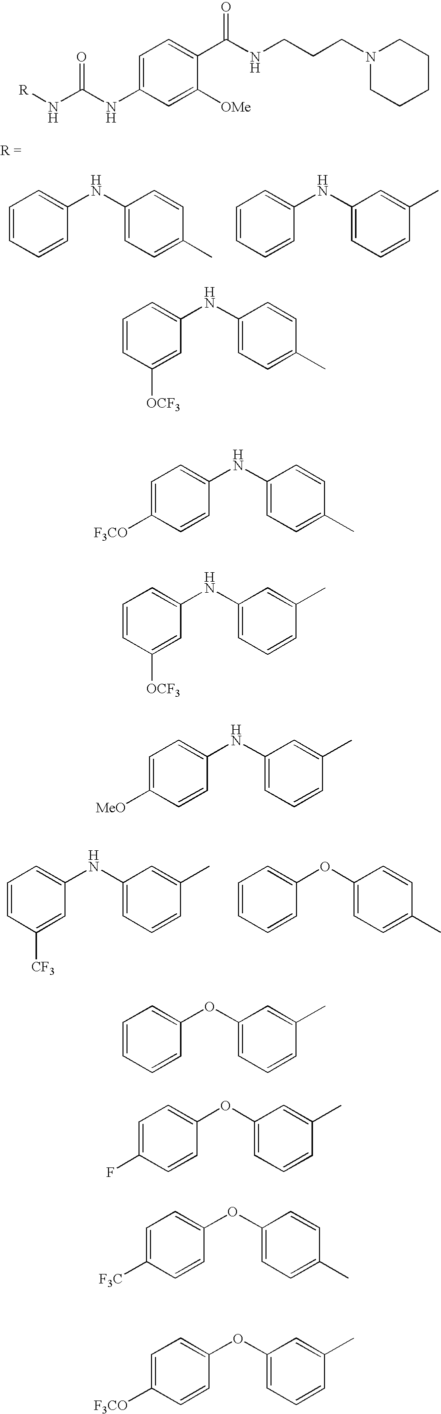

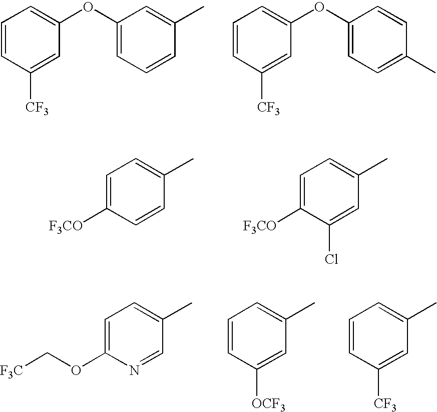

### Example 88

### 2-Methoxy-4-[3-(4-phenylamino-phenyl)-ureido]-N-(3-piperidin-1-yl-propyl)-benzamide)

Ex 86 and 4-Phenylamino-benzoic acid were coupled giving 41 mg (54%) of the title product. LCMS (an20p15): RT=6.05 min, (M+1)=502.

### Example 89

### 2-Methoxy-4-[3-(3-phenylamino-phenyl)-ureido]-N-(3-piperidin-1-yl-propyl)-benzamide

Ex 86 and 3-Phenylamino-benzoic acid were coupled giving 53 mg (71%) of the title product. LCMS (an20p15): RT=5.44 min, (M+1)=502.

### Example 90

### 2-Methoxy-N-(3-piperidin-1-yl-propyl)-4-{3-[4-(3-trifluoromethoxy-phenylamino)-phenyl]-ureido}-benzamide

Ex 86 and 4-(3-trifluoromethoxy-phenylamino)-benzoic acid were coupled giving 46 mg (52%) of the title product. LCMS (an20p15): RT=6.83 min, (M+1)=586.

### Example 91

### 2-Methoxy-N-(3-piperidin-1-yl-propyl)-4-{3-[4-(4-trifluoromethoxy-phenylamino)-phenyl]-ureido}-benzamide

Ex 86 and 4-(4-trifluoromethoxy-phenylamino)-benzoic acid were coupled giving 67 mg (76%) of the title product. LCMS (an20p15): RT=6.89 min, (M+1)=586.

### Example 92

### 2-Methoxy-N-(3-piperidin-1-yl-propyl)-4-{3-[3-(3-trifluoromethoxy-phenylamino)-phenyl]-ureido}-benzamide

Ex 86 and 3-(3-Trifluoromethoxy-phenylamino)-benzoic acid were coupled giving 50 mg (60%) of the title product. LCMS (an20p15): RT=6.24 min, (M+1)=586.

### Example 93

### 2-Methoxy-4-{3-[3-(4-methoxy-phenylamino)-phenyl]-ureido}-N-(3-piperidin-1-yl-propyl)-benzamide

Ex 86 and 4-(4-Methoxy-phenylamino)-benzoic acid were coupled giving 20 mg (25%) of the title product. LCMS (an20p10): RT=5.77 min, (M+1)=532.

### Example 94

### 2-Methoxy-N-(3-piperidin-1-yl-propyl)-4-{3-[3-(4-trifluoromethyl-phenylamino)-phenyl]-ureido}-benzamide

Ex 86 and 3-(3-Trifluoromethyl-phenylamino)-benzoic acid were coupled giving 30 mg (35%) of the title product. LCMS (an20p10): RT=6.19 min, (M+1)=570.

### Example 95

### 2-Methoxy-4-[3-(4-phenoxy-phenyl)-ureido]-N-(3-piperidin-1-yl-propyl)-benzamide

Ex 86 and 4-phenoxy-phenyl isocyanate were coupled giving 22.6 mg (23%) of the title product. LCMS (an20p10): RT=5.84 min, (M+1)=503.

### Example 96

### 2-Methoxy-4-[3-(3-phenoxy-phenyl)-ureido]-N-(3-piperidin-1-yl-propyl)-benzamide

Ex 86 and 3-phenoxyphenyl isocyanate were coupled giving 69 mg (68%) of the title product. LCMS (an20p15): RT=6.55 min, (M+1)=503.

### Example 97

### 4-{3-[3-(4-Fluoro-phenoxy)-phenyl]-ureido}-2-methoxy-N-(3-piperidin-1-yl-propyl)-benzamide

Ex 86 and 4-(4′-fluorophenoxy)benzoic acid were coupled giving the title product. LCMS (an20p10): RT=6.06 min, (M+1)=521.

### Example 98

### 2-Methoxy-N-(3-piperidin-1-yl-propyl)-4-{3-[4-(4-trifluoromethyl-phenoxy)-phenyl]-ureido}-benzamide

Ex 86 (86 mg, 0.30 mmol) and 4-(4′-trifluoromethylphenoxy)benzoic acid (160 mg, 0.57 mmol) were coupled giving 47 mg (27%) of the title product. LCMS (an20p15): RT=6.84 min, (M+1)=571. ¹H NMR (300 MHz, CD₃Cl): δ 1.86 (t, 2H), 3.90 (s, 3H), 6.70 (d, 1H), 8.21 (t, 1H), 9.13 (s, 1H), 9.33 (s, 1H).

### Example 99

### 2-Methoxy-N-(3-piperidin-1-yl-propyl)-4-{3-[4-(4-trifuoromethoxy-phenoxy)-phenyl]-ureido}-benzamide

Ex 86 and 4-(4-trifluoromethoxy-phenoxy)-benzoic acid were coupled giving 62 mg (71%) of the title product. LCMS (an20p15): RT=7.17 min, (M+1)=587.

### Example 100

### 2-Methoxy-N-(3-piperidin-1-yl-propyl)-4-{3-[3-(3-trifluoromethyl-phenoxy)-phenyl]-ureido}-benzamide

Ex 86 and 3-(3-trifluoromethyl-phenoxy)-benzoic acid were coupled giving 24 mg (28%) of the title product. LCMS (an20p10): RT=6.41 min, (M+1)=571.

### Example 101

### 2-Methoxy-N-(3-piperidin-1-yl-propyl)-4-{3-[4-(3-trifluoromethoxy-phenoxy)-phenyl]-ureido}-benzamide

Ex 86 and 4-(3-Methoxy-phenoxy)-benzoic acid were coupled giving 10 mg (11%) of the title product. LCMS (an20p15): RT=7.25 min, (M+1)=587.

### Example 102

### 2-Methoxy-N-(3-piperidin-1-yl-propyl)-4-[3-(4-trifluoromethyl-phenyl)-ureido]-benzamide

Ex 86 and 4-trifluoromethylphenyl isocyanate were coupled giving 15.5 mg (16%) of the title product. LCMS (an20p15): RT=6.16 min, (M+1)=479.

### Example 103

### 4-[3-(3-Chloro-4-trifluoromethoxy-phenyl)-ureido]-2-methoxy-N-(3-piperidin-1-yl-propyl)-benzamide

Ex 86 (0.14 g, 0.47 mmol) and 3-chloro-4-trifluoromethoxybenzoic acid (0.20 g, 0.83 mmol) were coupled giving 38 mg (%) of the title product. LCMS (an20p15): RT=6.54 min, (M+1)=529.

### Example 104

### 2-Methoxy-N-(3-piperidin-1-yl-propyl)-4-{3-[6-(2,2,2-trifluoro-ethoxy)-pyridin-3-yl]-ureido}-benzamide

Ex 86 and 6-(2,2,2-Trifluoro-ethoxy)-nicotinic acid were coupled giving 15 mg (20%) of the title product. LCMS (an20p15): RT=5.83 min, (M+1)=510.

### Example 105

### 2-Methoxy-N-(3-piperidin-1-yl-propyl)-4-[3-(3-trifluoromethoxy-phenyl)-ureido]-benzamide

Ex 86 and 3-trifluoromethoxybenzoic acid were coupled giving 13.3 mg (18%) of the title product. LCMS (an20p15): RT=7.55 min, (M+1)=495.

### Example 106

### 2-Methoxy-N-(3-piperidin-1-yl-propyl)-4-[3-(3-trifluoromethyl-phenyl)-ureido]-benzamide

Ex 86 and 3-trifluorotosyl isocyanate were coupled giving 36.7 mg (38%) of the title product. LCMS (an20p15): RT=6.19 min, (M+1)=479.

According to the procedure described hereby the following compounds were prepared: General procedure:

2-(3,5-Dimethoxy-4-formyl-phenoxy)ethyl polystyrene resin (200 mg, loading indicated by supplier 0.78 mmol/g, 0.16 mmol) was placed in a 12 mL fritted Teflon reactor fixed on an orbital shaker. A solution of amine (0.48 mmol, 3 eq) in NMP (1 mL), a solution of NaCNBH₃ (30 mg, 3 eq) in NMP (1 mL), AcOH (100 μL), and water (10 μL) was added to the resin. The mixture was shaken at room temperature over night. The resin was washed according to the general washing procedure described below. General washing procedure: NMP (2 mL), DCM (2 mL), MeOH (2 mL), DCM (2 mL), and NMP (2 mL).

A solution of 2-methoxy-4-nitrobenzoic acid (99 mg, 3 eq) and 1-hydroxybenzotriazole (68 mg, 3 eq) in NMP/DCM (1 mL/1 mL) was added. Diisopropylcarbodiimide (80 μL, 3 eq) and diisopropylethylamine (30 μL, 1 eq) was added. The mixture was shaken at room temperature for 4 h, and hereafter the resin was washed according to the standard procedure. A solution of SnCl₂.H₂O (180 mg, 5 eq) in NMP (2 mL) was added and diisopropylethylamine (30 μL, 1 eq) was added. The mixture was shaken over night at room temperature, and hereafter the resin was washed with NMP (2 mL), DCM (2 mL), MeOH (2 mL), 2×DCM (2 mL). The resin was treated with a solution of 3-phenoxyphenyl isocyanate (65 μL, 3 eq) in dry DCM (2 mL) and shaken at room temperature for 3 h. The resin was washed with NMP (2 mL), DCM (2 mL), MeOH (2 mL), 2×DCM (2 mL), and the treatment with 3-phenoxyphenyl isocyanate (65 μL, 3 eq) in dry DCM (2 mL) was repeated. Finally the resin was washed with NMP (2 mL), 5% diisopropylamine in NMP (2 mL), NMP (2 mL), DCM (2 mL), 5% AcOH in DCM (2 mL), DCM (2 mL), MeOH (2 mL), 3×DCM (2 mL). The resin was treated with TFA/DCM/TES (60:35:5, v/v, 2 mL) for 2 h at room temperature and hereafter washed with DCM (1 mL) to cleave the product from the resin. The samples were evaporated, redissolved in water/acetonitrile (2:8, v/v, 1 mL) and purified by preparative LC-MS. The compounds were eluted over 20 min with 20-95% acetonitrile in water (both solvents contained 0.01% TFA or 0.01% formic acid).

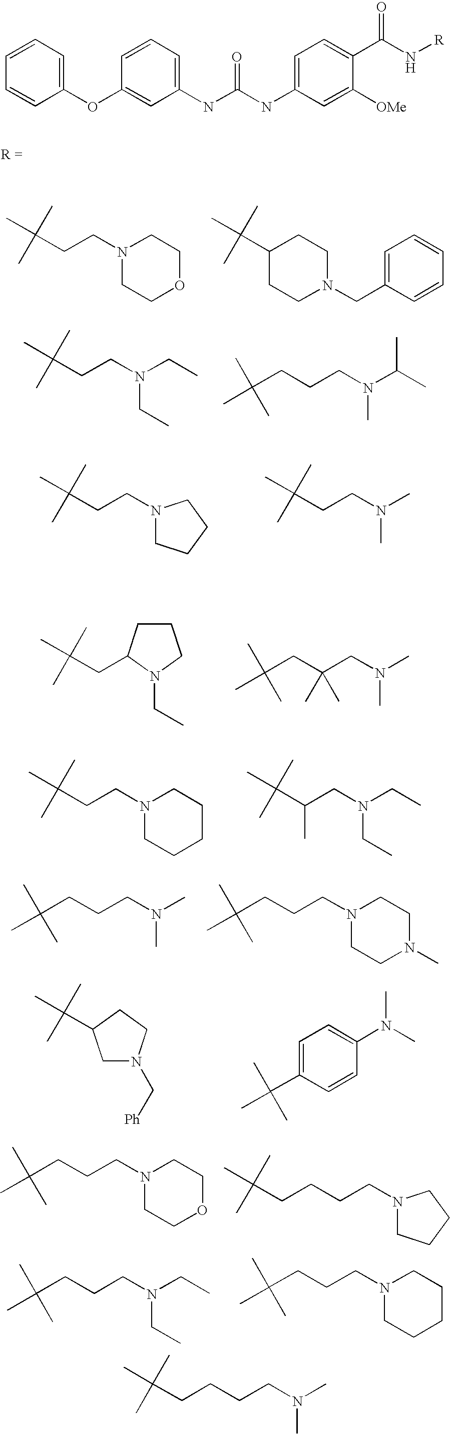

### Example 107

### 2-Methoxy-N-(2-morpholin-4-yl-ethyl)-4-[3-(3-phenoxy-phenyl)-ureido]-benzamide

From the reaction was 91 mg of the crude product isolated giving after purification 13 mg (13%) of the title product. ¹H-NMR (CDCl₃): δ 10.98 (s, 1H, NH⁺), 8.49 (s, 1H), 8.23 (s, 1H), 8.08 (s, 1H), 7.72 (d, 1H, J=7.7 Hz), 7.26-6.59 (m, 11H), 3.91 (d, 2H, J=12 Hz), 3.72 (s, 3H, OCH₃), 3.68 (s, 4H), 3.54 (d, 2H, J=12 Hz), 3.23 (s, 2H), 2.88 (br t, 2H). LCMS(an10p15): RT=8.39 min, (M+1)=491.

### Example 108

### N-(1-Benzyl-piperidin-4-yl)-2-methoxy-4-[3-(3-phenoxy-phenyl)-ureido]-benzamide

From the reaction was 161 mg of the crude product isolated giving after purification 37 mg (34%) of the title product. ¹H-NMR (CDCl₃): δ 10.04 (s, 1H, NH⁺), 8.41 (s, 1H), 8.20 (s, 1H), 7.83 (d, 1H, J=6.8 Hz), 7.66 (d, 1H, J=7.7 Hz), 7.32-6.52 (m, 16H), 3.97 (s, 3H), 3.67 (s, 3H, OCH₃), 3.35 (s, 2H), 2.64 (s, 2H), 2.01 (s, 2H), 1.81 (br s, 2H). LCMS(an10p15): RT=9.05 min, (M+1)=551.

### Example 109

### N-(2-Diethylamino-ethyl)-2-methoxy-4-[3-(3-phenoxy-phenyl)-ureido]-benzamide

From the reaction was 97 mg of the crude product isolated giving after purification 6.6 mg (9%) of the title product. ¹H-NMR (CDCl₃): δ 9.43 (s, 1H), 9.20 (s, 1H), 8.45 (m, 2H), 7.78 (d, J=8.5 Hz, 1H), 7.47 (s, 1H), 7.39-6.39 (m, 9H), 3.84 (s, 3H, OCH₃), 3.78 (br s, 2H), 3.21 (m, 2H), 3.14 (m, 4H), 1.32 (t, J=7.4 Hz, 6H). LCMS(an10p15): RT=5.79 min, (M+1)=477, RT=7.19 min, (M+1)=186, 20% (aniline from isocyanate)

### Example 110

### N-[3-(Isopropyl-methyl-amino)-propyl]-2-methoxy-4-[3-(3-phenoxy-phenyl)-ureido]-benzamide

To a solution of 2-methoxy-4-[3-(3-phenoxy-phenyl)-ureido]-benzoic acid (80 mg, 0.2 mmol), prepared using the same procedure as in example 45, in dichloromethane (20 mL) were HOBT (38 mg, 0.27 mmol), EDAC (61 mg, 0.32 mmol), N¹-isopropyl-N¹-methyl-propyldiamine hydrochloride (52 mg, 0.25 mmol) and diisopropylethylamine (66 mg, 88 μL, 0.5 mmol) added and the stirring continued at room temperature over night. EtOAc was added to the reaction mixture and the organic phase was washed with NaHCO3 (sat). The aqueous phase was extracted with EtOAc and the combined organic phases was dried (MgSO₄) and concentrated giving the crude product. The crude product was chromatographed (silica, CH₂Cl₂/methanol, 85:15) followed by a SCX-colon and preparative LCMS giving 2.2 mg of the title product. ¹H NMR (300 MHz, CD₃Cl): δ 3.85 (s, 3H), 8.19 (t, 1H), 9.34 (s, 1H), 9.59 (s, 1H). LCMS (an20p15): (M+1)=491.

### Example 111

### 2-Methoxy-4-[3-(3-phenoxy-phenyl)-ureido]-N-(2-pyrrolidin-1-yl-ethyl)-benzamide

From the reaction was 111 mg of the crude product isolated giving after purification 18 mg (20%) of the title product. ¹H-NMR (CDCl₃): δ 10.71 (s, 1H), 8.74 (s, 1H), 8.49 (s, 1H), 8.29 (s, 1H), 7.65 (s, 1H), 7.34-6.95 (m, 11H), 3.65 (s, 3H), 3.62 (s, 4H), 3.18 (s, 2H), 2.75 (s, 2H), 1.92 (s, 4H). LCMS(an10p15): RT=8.51 min, (M+1)=475.

### Example 112

### N-(2-Dimethylamino-ethyl)-2-methoxy-4-[3-(3-phenoxy-phenyl)-uraido]-benzamide

From the reaction was 113 mg of the crude product isolated giving after purification 21 mg (23%) of the title product. ¹H-NMR (CDCl₃): δ 10.68 (s, 1H, NH⁺), 8.70 (s, 1H), 8.42 (br s, 1H), 8.31 (s, 1H), 7.67 (d, 1H, J=7.5 Hz), 7.31-6.52 (m, 11H), 3.69 (s, 3H, OCH₃), 3.59 (s, 2H), 3.31 (s, 2H), 2.76 (s, 6H). LC-MS(an20p15): RT=8.30 min, (M+1)=449.

### Example 113

### N-(1-Ethyl-pyrrolidin-2-ylmethyl)-2-methoxy-4-[3-(3-phenoxy-phenyl)-ureido]-benzamide

From the reaction was 124 mg of the crude product isolated giving after purification 10 mg (13%) of the title product. ¹H-NMR (CDCl₃): δ 9.43 (s, 1H), 9.20 (s, 1H), 8.60 (t, 1H), 7.77 (d, J=8.7 Hz, 1H), 7.47 (s, 1H), 7.35-6.85 (m, 9H), 6.59 (d, 1H), 3.83 (s, 3H, OCH₃), 3.59 (m, 4H), 3.21 (m, 1H), 2.81 (m, 2H), 2.13 (m, 1H), 1.96 (m, 2H), 1.82 (m, 1H), 1.26 (t, J=7.2 Hz, 3H). LCMS(an10p15): RT=5.90 min, (M+1)=489.

### Example 114

### N-(3-Dimethylamino-2,2-dimethyl-propyl)-2-methoxy-4-[3-(3-phenoxy-phenyl)-ureido]-benzamide

To a solution of 4-amino-N-(3-dimethylamino-2,2-dimethyl-propyl)-2-methoxy-benzamide (25 mg, 0.09 mmol), prepared according to the procedure for example 97, in dichloromethane (3 mL) was 3-phenoxyphenyl isocyanate (37 mg, 32 μL, 0.18 mmol) added and the reaction was stirred under inert atmosphere over night. The solvent was removed in vacuo. The crude product was chromatographed (silica, CH₂Cl₂/methanol, 92:8) giving 37 mg of the title product. ¹H NMR (300 MHz, CD₃Cl): δ 3.92 (s, 3H), 8.82 (t, 1H), 8.74 (s, 1H), 8.94 (s, 1H).

### Example 115

### 2-Methoxy-4-[3-(3-phenoxy-phenyl)-ureido]-N-(2-piperidin-1-yl-ethyl)-benzamide

From the reaction was 90 mg of the crude product isolated giving after purification 18 mg (18%) of the title product. ¹H-NMR (CDCl₃): δ 10.72 (s, 1H, NH⁺), 9.09 (s, 1H), 8.80 (s, 1H), 8.33 (s, 1H), 7.67 (s, 1H), 7.27-6.76 (m, 10H), 6.52 (d, 1H, J=7.2 Hz), 3.69 (s, 3H, OCH₃), 3.58 (s, 2H), 3.41 (s, 2H), 3.04 (s, 2H), 2.58 (s, 2H), 1.73 (s, 4H), 1.27 (s, 2H). LCMS(an10p15): RT=8.76 min, (M+1)=489.

### Example 116

### N-(2-Diethylamino-1-methyl-ethyl)-2-methoxy-4-[3-(3-phenoxy-phenyl)-ureido]-benzamide

From the reaction was 118 mg of the crude product isolated giving after purification 17 mg (17%) of the title product. ¹H-NMR (CDCl₃): δ 10.18 (s, 1H, NH⁺), 9.04 (s, 1H), 8.72 (s, 1H), 8.27 (s, 1H), 7.69 (br d, 1H), 7.32-6.50 (m, 11H), 3.74 (s, 3H, OCH₃), 3.36 (s, 2H), 2.99 (brs, 4H), 2.90 (s, 3H), 1.88 (s, 1H), 1.16 (t, 6H, J=7.1H Hz). LCMS(an10p15): RT=8.65 min, (M+1)=491.

### Example 117

### N-(3-Dimethylamino-propyl)-2-methoxy-4-[3-(3-phenoxy-phenyl)-ureido]-benzamide

From the reaction was 117 mg of the crude product isolated giving after purification 15 mg (16%) of the title product. ¹H-NMR (CDCl₃): δ 10.86 (s, 1H, NH⁺), 8.99 (s, 1H), 8.69 (s, 1H), 8.16 (s, 1H), 7.65 (d, 1H, J=7.5 Hz), 7.31-6.54 (m, 11H), 3.73 (s, 3H, OCH₃), 3.35 (s, 2H), 2.91 (s, 2H), 2.67 (s, 6H), 1.88 (s, 2H). LCMS(an10p15): RT=8.31 min, (M+1)=463.

### Example 118

### 2-Methoxy-N-[3-(4-methyl-piperazin-1-yl)-propyl]-4-[3-(3-phenoxy-phenyl)-ureido]-benzamide

From the reaction was 113 mg of the crude product isolated giving after purification 8.0 mg (7%) of the title product. ¹H-NMR (CDCl₃): δ 9.92 (s, 1H), 9.84 (s, 1H,), 8.81 (t, 1H, J=205.7 Hz, NHCO), 8.40 (d, 1H, J=8.5 Hz), 8.08-7.61 (m, 11H, m), 7.27 (d, 1H, J=7.5 Hz), 4.51 (s, 6H, OCH₃+CH₃), 3.97 (br m, 6H), 3.63 (s, 2H), 3.43 (s, 4H), 3.31 (s, 2H). LCMS(an10p15): RT=7.56 min, (M+1)=518.

### Example 119

### N-(1-Benzyl-pyrrolidin-3-yl)-2-methoxy-4-[3-(3-phenoxy-phenyl)-ureido]-benzamide

From the reaction was 167 mg of the crude product isolated giving after purification 23 mg (22%) of the title product. ¹H-NMR (CDCl₃): δ 11.61 (s, 1H, NH⁺), 8.81 (s, 1H), 8.57 (s, 1H), 8.34 (s, 1H), 7.62 (br d, 1H), 7.25-6.49 (m, 16H), 4.55 (s, 1H), 4.05 (s, 3H, OCH₃), 3.06 (d, 4H), 3.36 (s, 1H), 3.13 (s, 1H), 2.79 (s, 1H), 2.37 (s, 1H). LCMS(an10p15): RT=9.30 min, (M+1)=537.

### Example 120

### N-(4-Dimethylamino-phenyl)-2-methoxy-4-[3-(3-phenoxy-phenyl)-ureido]-benzamide

From the reaction was 122 mg of the crude product isolated giving after purification 25 mg (25%) of the title product. ¹H-NMR (CDCl₃): δ 9.14 (s, 1H, NH⁺), 8.53 (s, 1H), 8.21 (s, 1H), 7.73 (d, 1H, H=9.0 Hz), 7.51 (d, 1H, J=8.7 Hz), 7.36-6.54 (m, 15H), 3.79 (s, 3H, OCH₃), 3.03 (s, 6H). LCMS(an10p15): RT=8.83 min, M+1=497.

### Example 121

### 2-Methoxy-N-(3-morpholin-4-yl-propyl)-4-[3-(3-phenoxy-phenyl)-ureido]-benzamide

The title product was prepared according to the procedure described in Ex 62, giving after purification an isolated yield of 16.7 mg. ¹H-NMR (dmso-d₆): δ 3.89 (s, 3H), 8.04 (s, 1H), 9.09 (s, 1H), 9.12 (s, 1H). LCMS(an10p15): (M+1)=497.

### Example 122

### 2-Methoxy-4-[3-(3-phenoxy-phenyl)-ureido]-N-(4-pyrrolidin-1-yl-butyl)-benzamide

From the reaction was 93 mg of the crude product isolated giving after purification 18 mg (20%) of the title product. ¹H-NMR (CDCl₃): δ 9.33 (s, 1H), 9.13 (s, 1H), 8.65 (s, 1H), 7.94 (t, J=5.9 Hz, 1H), 7.85 (d, J=8.7 Hz, 1H), 7.57 (s, 1H), 7.34-6.61 (m, 9H), 3.86 (s, 3H, OCH₃), 3.36 (m, 2H), 3.13 (brs, 4H), 2.99 (m, 2H), 1.98 (brs, 4H), 1.73 (m, 2H), 1.56 (m, 2H). LCMS(an20p10): RT=5.76 min, (M+1)=503.

### Example 123

### N-(3-Diethylamino-propyl)-2-methoxy-4-[3-(3-phenoxy-phenyl)-ureido]-benzamide

From the reaction was 94 mg of the crude product isolated giving after purification 18 mg (20%) of the title product. ¹H-NMR (CDCl₃): δ 9.65 (s, 1H), 9.39 (s, 1H), 8.21 (t, 1H), 7.81 (d, 1H), 7.50 (s, 1H), 7.22-6.85 (m, 9H), 6.58 (d, 1H), 3.83 (s, 3H, OCH₃), 3.45 (br q, 2H), 3.05-2.91 (br m, 6H), 1.95 (br t, 2H), 1.23 (t, 6H, J=7.2 Hz). LCMS(an10p15): RT=8.62 min, (M+1)=491.

### Example 124

### N-(4-Dimethylamino-butyl)-2-methoxy-4-[3-(3-phenoxy-phenyl)-ureido]-benzamide

From the reaction was 99 mg of the crude product isolated giving after purification 8.4 mg (20%) of the title product. ¹H-NMR (CDCl₃): δ 9.13 (s, 1H), 8.88 (s, 1H), 7.98 (t, J=5.7 Hz, 1H), 7.80 (d, J=8.7 Hz, 1H), 7.28 (s, 1H), 7.32-6.60 (m, 10H), 3.83 (s, 3H, OCH₃), 3.35 (m, 2H), 2.93 (m, 2H), 2.66 (s, 6H), 2.02 (m, 2H), 1.56 (m, 2H). LCMS(an20p10): RT=5.57 min, (M+1)=477.

### Example 125

### N-(1-Benzyl-piperidin-4-yl)-2-methoxy-4-methylamino-benzamide

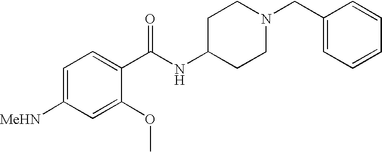

4-Amino-N-(1-benzyl-piperidin-4-yl)-2-methoxy-benzamide (synthesised according to the same procedure as example 97) was dissolved in methanol and sodium methoxide (5.7 equiv.) and paraformaldehyde (1.5 equiv.) were added. The reaction was stirred over night under inert atmosphere at 40° C. The mixture was cooled to room temperature whereupon sodium borohydride (2.4 equiv.) was added slowly and the reaction was continuously stirred over night at 50° C. The solvent was removed in vacuo. The residue was dissolved in NaHCO3-solution (150 mL), extracted with tert-butylmethylether (3×100 mL). The combined organic phases was dried (Na2SO4) and concentrated. The crude product was chromatographed (silica, dichloromethane/methanol/ammonia, 100:10:1) giving the title product (78%). ¹H NMR (300 MHz, CD₃Cl): δ 2.89 (d, 3H, —NHMe).

### Example 126

### 4-[3-(4-Benzyl-phenyl)-1-methyl-ureido]-N-(1-benzyl-piperidin-4-yl)-2-methoxy-benzamide

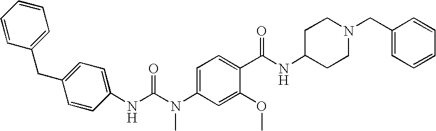

To a solution Ex 140 (20 mg, 0.057 mmol) in dichloromethane (0.5 mL) was 4-benzylphenyl isocyanate (24 mg, 0.11 mml) added and the flask was flushed with nitrogen. The reaction mixture was stirred for four days when PS-trisamine (3.56 mmol/g, 100 mg) was added. After two days was the resin filtered off and rinsed with dichloromethane. The reaction mixture was concentrated in vacuo. The crude product was purified with acidic ion exchange chromatography (SCX-colon) giving 28 mg (87%) of the title product. LCMS (an20p15): RT=6.75 min, (M+1)=563.

### Example 127

### N-(1-Benzyl-piperidin-4-yl)-4-[3-(9H-fluoren-2-yl)-1-methyl-ureido]-2-methoxy-benzamide

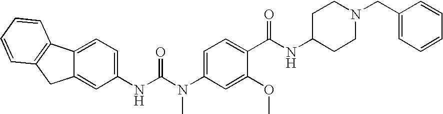

To a solution Ex 125 (20 mg, 0.057 mmol) in dichloromethane (0.5 mL) was 9H-fluoren-2-yl isocyanate (24 mg, 0.11 mml) added and the flask was flushed with nitrogen. The reaction mixture was stirred for four days when PS-trisamine (3.56 mmol/g, 100 mg) was added. After two days was the resin filtered off and rinsed with dichloromethane. The reaction mixture was concentrated in vacuo. The crude product was purified with acidic ion exchange chromatography (SCX-colon) giving 27 mg (85%) of the title product. LCMS (an20p15): RT=6.62 min, (M+1)=561.

### Example 128

### 2-Methoxy-4-{3-[5-(2-methyl-thiazol-4-yl)-thiophen-2-yl]-ureido}-N-(3-morpholin-4-yl-propyl)-benzamide

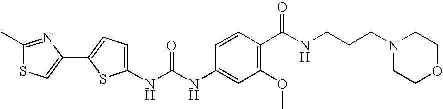

To a solution of 5-(2-methyl-1,3-thiazol-4-yl)thiophen-2-caboxylic acid (102 mg, 0.45 mmol) in toluene (5 mL) were dihenylphosphorylazid (79 μL, 0.37 mmol) and triethylamine (42 □L) added and therafter was the reaction mixture heated to reflux. After 3 h was Ex. 61 (67 mg, 0.23 mmol) dissolved in hot toluene (2 mL) added. The reaction was allowed cooled a bit before dichloromethane (2 mL) was added and thereafter was the reaction left over night. The solvent was removed in vacuo. The crude product was purified with acidic ion exchange chromatography (SCX-colon) followed by one more chromatography (silica, dichloromethane/methanol/ammoniak, 9:1+1%) giving 71 mg (61%) of the title product. ¹H NMR (300 MHz, dmso-d₆): δ 2.53 (s, 3H), 3.82 (s, 3H), 6.98 (dd, 1H), 8.07 (t, 1H), 9.08 (s, 1H), 9.83 (s, 1H).

### Example 129

### 4-[3-(4-Benzyloxy-phenyl)-1-methyl-ureido]-N-(1-benzyl-piperidin-4-yl)-2-methoxy-benzamide

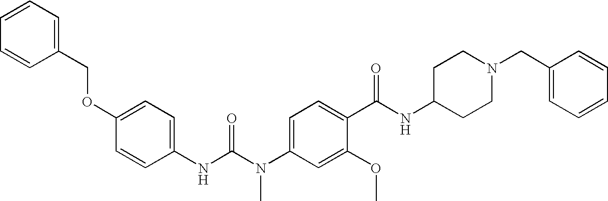

To a solution Ex 125 (20 mg, 0.057 mmol) in dichloromethane (0.5 mL) was 4-benzyloxy-phenyl isocyanate (24 mg, 0.11 mml) added and the flask was flushed with nitrogen. The reaction mixture was stirred for four days when PS-trisamine (3.56 mmol/g, 100 mg) was added. After two days was the resin filtered off and rinsed with dichloromethane. The reaction mixture was concentrated in vacuo. The crude product was purified with acidic ion exchange chromatography (SCX-colon) giving 28 mg (85%) of the title product. LCMS (an20p15): RT=5.73 min, (M+1)=579.

### Example 130

### 4-[3-(9H-Fluoren-2-yl)-ureido]-2-methoxy-N-(3-morpholin-4-yl-propyl)-benzamide)

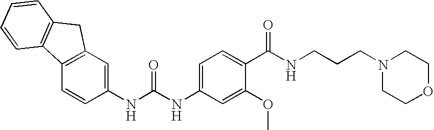

To a solution Ex 61 (20 mg, 0.068 mmol) in dichloromethane (0.5 mL) was 9H-fluoren-2-yl isocyanate (28 mg, 0.14 mml) added and the flask was flushed with nitrogen. The reaction mixture was stirred for four days when PS-tosyl chloride (1.0 equiv.) was added. After 12 h was the resin filtered off and rinsed with dichloromethane. The reaction mixture was concentrated in vacuo. The crude product was purified with chromatography (dichloromethane/methanol, 92:8) giving 8.5 mg (25%) of the title product. ¹H NMR (300 MHz, dmso-d₆): δ 3.40 (s, 3H), 8.05 (t, 1H), 8.87 (s, 1H), 9.02 (s, 1H). LCMS (an20p15): (M+1)=501 m/z.

### Example 131

### 2-Methoxy-4-[1-methyl-3-(4-phenoxy-phenyl)-ureido]-N-(3-morpholin-4-yl-propyl)-benzamide

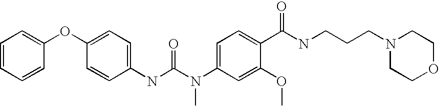

### Example 132

### 4-[3-(3-Chloro-phenyl)-ureido]-N-(2-diethylamino-ethyl)-2-methoxy-benzamide

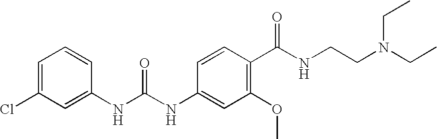

To a solution of 4-amino-N-(2-diethylamino-ethyl)-2-methoxy-benzamide (30 mg, 0.11 mmol) in dry dichloromethane (1.5 mL) was 3-chlorophenyl isocyanate (28 μL, 0.22 mmol) added and the reaction was stirred three days under inert atmosphere. PS-Trisamine (100 mg, 3.58 mmol/g) was added and after gentle stirring for 2 h, and addition of methanol (2 mL), was the resin removed by filtration. The resin was washed with dichloromethane (2 mL). The solvents were removed in vacuo and the crude product was purified through chromatography (silica, CH₂Cl₂/methanol/ammoniak, 101:10:1) giving the desired product. ¹H-NMR (dmso-d₆): δ 0.99 (t, 4H), 2.20 (s, 2H), 3.90 (s, 3H), 8.98 (s, 1H), 9.07 (s, 1H). Mass analysis; found (M+1)=419.

### Example 133

### N-(2-Diethylamino-ethyl)-2-ethoxy-4-[3-(4-phenoxy-phenyl)-ureido]-benzamide

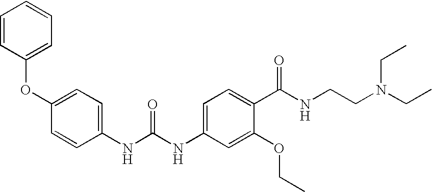

A solution of methyl-4-acetamido-2-ethoxybenzoate (1 g, 4.2 mmol) and lithium hydroxide (0.5 g, 21 mmol) in a THF/water mixture (50 ml/25 ml) was heated to 70° C. for 18 h. After cooling, solvent was removed in vacuo to give a white semi-solid (0.788 g, 4.2 mmol, 100%). ¹H NMR (300 MHz, D₂O): δ 1.2 (t, 3H), 3.95 (q, 2H), 6.25 (d, 1H), 6.35 (s, 1H), 7.2 (d, 1H).

To a cooled (0° C.) solution of 4-amino-2-ethoxybenzoic acid, lithium salt (0.78 g, 4.17 mmol) in a dioxane/water mixture (50 ml/25 ml) was added BOC₂O (0.92 g, 4.17 mmol). After stirring for 10 minutes at 0° C., the reaction mixture was stirred at RT for 4 h. The mixture was then cooled to 0° C. and further BOC₂O (1.84 g, 8.34 mmol) was added. After stirring for an additional 10 minutes at 0° C., the reaction mixture was stirred at RT for 2 days. Dioxane was removed in vacuo. The aqueous phase was diluted with water and washed with dichloromethane (3×). The aqueous phase was then saturated with NaCl, acidified with a 1N aq. HCl solution and quickly extracted with dichloromethane (3×). The organic phases were combined, washed with brine, dried over MgSO₄ and concentrated in vacuo to give a white solid as 4-Amino-2-ethoxy-benzoic acid (0.84 g, 2.96 mmol, 71%). ¹H NMR (300 MHz, CDCl₃): δ 1.53 (s, 9H), 1.55 (t, 3H), 4.35 (q, 2H), 6.69 (d, 1H), 6.75 (bs, 1H), 7.76 (s, 1H), 8.06 (d, 1H), 10.8 (bs, 1H)

To a solution of 4-Amino-2-ethoxy-benzoic acid (0.1 g, 0.35 mmol) in dichloromethane (20 ml) were added EDAC (0102 g, 0.53 mmol) and HOBt (0.062 g, 0.46 mmol). After stirring for 5 minutes, N,N-diethyl-ethylene diamine (60 μl, 0.43 mmol) was added and the reaction mixture was stirred at RT overnight. The mixture was washed with sat. aq. NaHCO₃ (3×), brine (2×), dried over MgSO₄ and concentrated in vacuo to give a colourless oil (0.135 g, 0.35 mmol, 100%). The oil was stirred overnight at RT in a TFA/dichloromethane mixture (3 ml/3 ml). Solvent was removed in vacuo. The residue was diluted with water and washed with dichloromethane (3×). The aqueous phase was saturated with NaCl and solid K₂CO₃ was added up to pH=12. The aqueous phase was extracted with dichloromethane (3×), the organic phases were combined, washed with brine, dried over MgSO₄ and concentrated in vacuo to give a pale-brown oil as 4-Amino-N-(2-diethylamino-ethyl)-2-ethoxy-benzamide (0.086 g, 0.31 mmol, 90%). ¹H NMR (300 MHz, CDCl₃): 31.04 (bt, 6H), 1.5 (t, 3H), 2.62 (bm,6H), 3.5 (bm, 2H), 3.92 (bs, 2H), 4.12 (q, 2H), 6.18 (s, 1H), 6.32 (d, 1H), 8.03 (d, 1H), 8.2 (bs, 1H)

A solution of 4-Amino-N-(2-diethylaminoethyl)-2-ethoxy-benzamide (0.08 g, 0.286 mmol) and 4-phenoxyphenyl isocyanate (77.6 μl, 0.429 mmol) in dichloromethane (5 ml) was stirred at RT overnight under an argon atmosphere. PS-trisamine (0.286 mmol) was added and the reaction mixture was stirred for a further 18 h00. Methanol (1 ml) was added to dissolve the precipitate. The resin was filtered off and the filtrate was concentrated to give a semi-solid which was triturated with methanol. The solid was filtered, washed with methanol and dried in vacuo to give a white powder (0.08 g, 0.163 mmol, 57%). ¹H NMR (300 MHz, DMSO): δ 0.97 (t, 6H), 1.45 (t, 3H), 2.53 (m, 6H), 3.35 (q, 2H), 4.18 (q, 2H), 6.95-7.5 (m, 11H), 7.85 (d, 1H), 8.2 (bm, 1H), 8.76 (s, 1H), 8.94 (s, 1H)

### Example 134

### N-(3-Dibutylamino-propyl)-2-methoxy-4-[1-methyl-3-(4-phenoxy-phenyl)-ureido]-benzamide

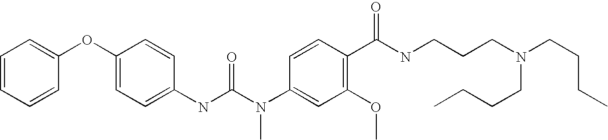

¹H-NMR (CDCl₃): δ 0.90 (m, 6H), 1.33 (m, 8H), 1.80 (m, 2H), 2.47 (m, 6H), 3.37 (s, 3H), 3.53 (q, 2H), 3.98 (s, 3H), 6.40 (s, 1H), 6.95-7.06 (m, 7H), 7.32 (m, 4H), 7.98 (bs, 1H), 8.23 (d, 1H)

### Example 135

### 4-{3-[4-(4-Fluoro-phenoxy)-phenyl]-ureido}-2-methoxy-N-(3-morpholin-4-yl-propyl)-benzamide

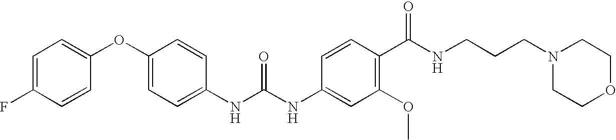

¹H-NMR (CDCl₃): δ 1.80(m 2H), 2.42(m 6H), 3.51 (m 2H), 3.66(m 4H), 3.93(s 3H), 6.45(m 1H), 6.91-7.04(m 6H), 7.28(s 1H), 7.39(m 1H), 7.90(m 2H), 8.20(m 1H), 8.48(s 1H), 8.77(s 1H).

### Example 136

### 2-Methoxy-N-(3-morpholin-4-yl-propyl)-4-(3-[4-(pyridin-2-yloxy)-phenyl]-ureido)-benzamide

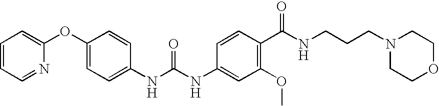

¹H-NMR (CDCl₃): δ 1.82 (m, 2H), 2.41 (m, 6H), 3.54-3.67 (m, 6H), 3.95 (s, 3H), 6.47 (dd, 1H), 6.87 (d, 1H), 7.0 (m, 1H), 7.09 (m, 2H), 7.47 (m, 2H), 7.68 (m, 1H), 7.92 (m, 2H), 8.18 (m, 2H), 8.53 (s, 1H), 8.75 (s, 1H)

### Example 137

### N-(1-Benzyl-piperidin-4-yl)-4-(3-indan-5-yl-ureido)-2-methoxy-benzamide

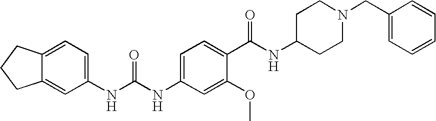

### Example 138

### 2-Methoxy-4-[3-(4-trifluoromethoxy-phenyl)-ureido]-benzoic acid

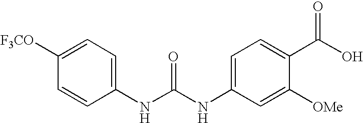

Using the same procedure as described in Ex 45 was the title product synthesised from 4-amino-2-methoxy-benzoic acid and 4-trifluoromethoxyphenyl isocyanate giving the title product.

### Example 139

### 2-Methoxy-N-(3-morpholin-4-yl-propyl)-4-{3-[4-(4-trifluoromethyl-phenylamino)-phenyl]-ureido}-benzamide

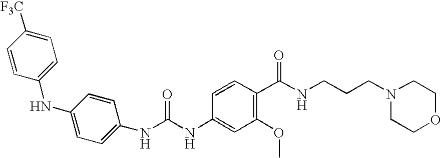

Following the same general procedure as described in Ex 88 was Ex 61 (57 mg, 0.20 mmol) and 4-(4-Trifluoromethyl-phenylamino)-benzoic acid (0.10 g, 0.36 mmol) giving 52 mg (45%) of the title product. LCMS (an20p15): (M+1)=572 m/z.

### Example 140

### 4-[3-(4-Bromo-phenyl)-ureido]-N-(2-diethylamino-ethyl)-2-methoxy-benzamide

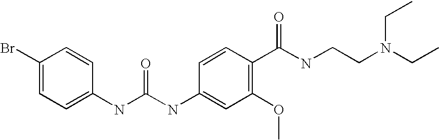

Using the same procedure as described in Ex 64 was the title product synthesised from Ex 63 and 4-Bromophenyl isocyanate.

NMR(DMSO-d₆): δ 0.99 (t, 6H), 3.29 (m, 2H), 3.89 (s, 3H), 8.25 (t, 1H), 8.91(s, 1H), 9.02 (s, 1H)

LC-MS (an20p10); Rt=5.33 min. (M+1)=464.9 m/z

### Example 141

### 4-[3-(3-Chloro-4-fluoro-phenyl)-ureido]-N-(2-diethylamino-ethyl)-2-methoxy-benzamide

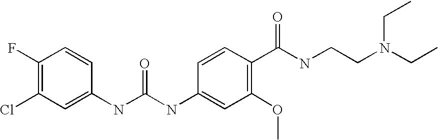

Using the same procedure as described in Ex 64 was the title product synthesised from Ex 63 and 3-Chloro-4-fluoro-phenyl isocyanate.

NMR(DMSO-d₆): δ 0.99 (t, 6H), 3.90 (s, 3H), 8.25 (t, 1H), 8.97 (s, 1H), 9.08 (s, 1H)

### Example 142

### 4-[3-(3,4-Dichloro-phenyl)-ureido]-N-(2-diethylamino-ethyl)-2-methoxy-benzamide

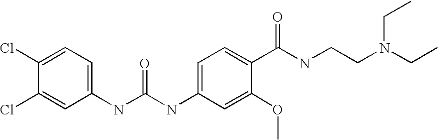

Using the same procedure as described in Ex 64 was the title product synthesised from Ex 63 and 3,4-Dichloro-phenyl isocyanate.

LCMS(an20p10); Rt=5.63 min. (M+1)=453 m/z

¹H NMR (DMSO-de): δ 10.36 (s, 1H), 10.32 (s, 1H), 8.26 (s, 1H), 7.93 (s, 1H), 7.80 (d, 1H), 7.52-7.44 (m, 3H), 7.04 (d, 1H), 3.89 (s, 3H), 3.40 (m, 2H), 2.65 (m, 6H), 1.03 (t, 6H)

### Example 143

### 4-[3-(3,5-Dichloro-phenyl)-ureido]-N-(2-diethylamino-ethyl)-2-methoxy-benzamide

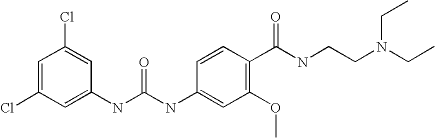

Using the same procedure as described in Ex 64 was the title product synthesised from Ex 63 and 3,5-Dichloro-phenyl isocyanate.

NMR(CDCl₃): δ 1.43 (t, 6H), 3.86 (m, 2H), 3.88 (s, 3H), 8.44 (m, 1H), 9.44 (s, 1H), 9.50 (s, 1H)

LC-MS(an20p10): Rt=5.75 min. (M+1)=454.9 m/z

### Example 144

### 4-[3-(4-Cyano-phenyl)-ureido]-N-(2-diethylamino-ethyl)-2-methoxy-benzamide

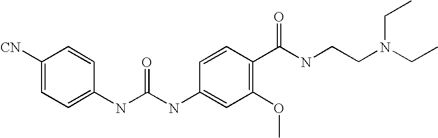

Using the same procedure as described in Ex 64 was the title product synthesised from

Ex 63 and 4-Cyano-phenyl isocyanate.

NMR(DMSO-d₆): δ 0.99 (t, 6H), 3.90 (s, 3H), 8.25 (t, 1H), 9.17 (s, 1H), 9.29 (s, 1H)

### Example 145

### 4-[3-(3-Chloro-4-methoxy-phenyl)-ureido]-N-(2-diethylamino-ethyl)-2-methoxy-benzamide

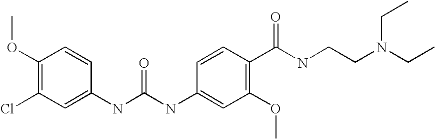

Using the same procedure as described in Ex 64 was the title product synthesised from Ex 63 and 3-Chloro-4-methoxy-phenyl isocyanate.

NMR(CDCl3): δ 1.31 (t, 6H), 3.73 (q, 2H), 3.85 (s, 3H), 8.39 (t, 1H), 9.22 (s, 1H), 9.46 (s, 1H)

LC-MS(an20p10): Rt=4.92 min. (M+1)=450.0 m/z

### Example 146

### N-(2-Diethylamino-ethyl)-2-methoxy-4-[2-(4-trifluoromethoxy-phenyl)-acetylamino]-benzamide

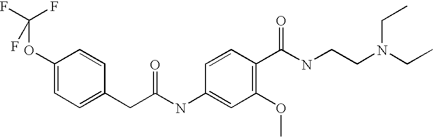

Using the same procedure as described in Ex 5 was the title product synthesised from Ex 63 and 4-(trifluoromethoxy)phenylacetic acid.

NMR(CDCl3): δ 1.03 (t, 6H), 3.78 (s, 2H), 3.91 (s, 3H), 8.47 (t, 1H), 8.62 (s, 1H)

### Example 147

### 4-[3-(2-Bromo-4-trifluoromethoxy-phenyl)-ureido]-N-(2-diethylamino-ethyl)-2-methoxy-benzamide

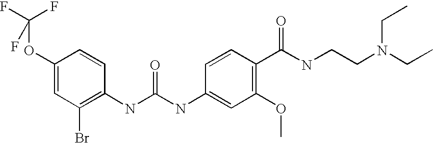

Using the same procedure as described in Ex 33 was the title product synthesised from Ex 170 and N*1*,N*1*-Diethyl-ethane-1,2-diamine.

NMR(CDCl₃): δ 1.03 (t, 6H), 3.91 (s, 3H), 8.33 (s, 1H), 9.83 (s, 1H)

LC-MS(an20p10): Rt=6.10 min. (M+1)=548.0 m/z

### Example 148

### N-(2-Diethylamino-ethyl)-2-methoxy-4-[3-(4-trifluoromethylsulfanyl-phenyl)-ureido]-benzamide

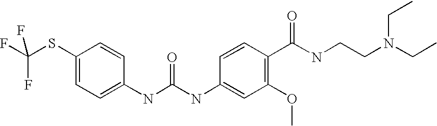

Using the same procedure as described in Ex 64 was the title product synthesised from Ex 63 and 4-trifluoromethylsulfanyl-phenyl isocyanate.

NMR(DMSO-d₆): δ 0.99 (t, 6H), 3.90 (s, 3H), 8.25 (t, 1H), 9.12 (s, 1H), 9.16 (s, 1H)

### Example 149

### N-(2-Diethylamino-ethyl)-2-methoxy-4-[3-(4-methylsulfanyl-phenyl)-ureido]-benzamide

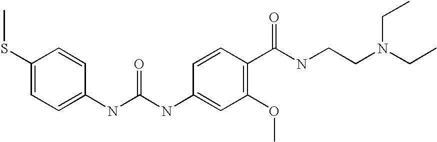

Using the same procedure as described in Ex 64 was the title product synthesised from Ex 63 and 4-methylsulfanyl-phenyl isocyanate.

NMR(DMSO-d₆): δ 0.99 (t, 6H), 3.89 (s, 3H), 8.25 (t, 1H), 8.79 (s, 1H), 8.99 (s, 1H)

### Example 150

### N-(2-Diethylamino-ethyl)-2-methoxy-4-[3-(3-methylsulfanyl-phenyl)-ureido]-benzamide

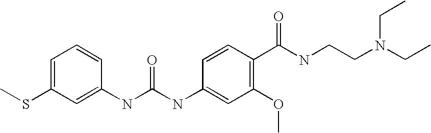

Using the same procedure as described in Ex 64 was the title product synthesised from Ex 63 and 3-methylsulfanyl-phenyl isocyanate.

NMR(CDCl₃): δ 1.26 (t, 6H), 2.45 (s, 3H), 3.68 (m, 2H), 3.84 (s, 3H), 8.38 (t, 1H), 9.35 (s, 1H), 9.57 (s, 1H)

LC-MS(an20p10): Rt=5.05 min. (M+1)=431.0 m/z

### Example 151

### 4-[3-(4-Chloro-3-trifluoromethyl-phenyl)-ureido]-N-(2-diethylamino-ethyl)-2-methoxy-benzamide

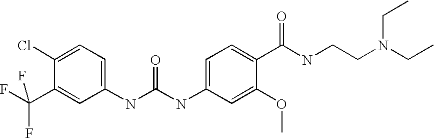

Using the same procedure as described in Ex 64 was the title product synthesised from Ex 63 and 4-Chloro-3-trifluoromethyl-phenyl isocyanate.

LCMS(an20p10); Rt=5.83 min. (M+1)=487 m/z

¹H-NMR (DMSO-d₆): δ 9.85 (s, 1H), 9.74 (s, 1H), 9.29 (br s, 1H), 8.39 (t, 1H), 8.15 (s, 1H), 7.81-7.52 (m, 4H), 7.05 (d, 1H), 3.90 (s, 3H), 3.64 (m, 2H), 3.23 (m, 6H), 1.22 (m, 6H).

### Example 152

### N-[3-(Cyclohexyl-ethyl-amino)-propyl]-2-methoxy-4-[3-(4-trifluoromethoxy-phenyl)-ureido]-benzamide

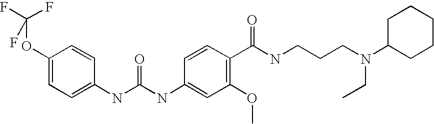

Using the same procedure as described in Ex 48 was the title product synthesised from Ex 138 and N*1*-Cyclohexyl-N*1*-ethyl-propane-1,3-diamine (synthesized as described in Ex 66 using Bromopropyl phtalimide and Cyclohexyl-ethyl-amine)

NMR(CDCl3): 0.95 (t,3H), 3.87 (s,3H), 8.05 (s, 1H), 9.17 (m, 2H)

### Example 153

### 2-Methoxy-4-[3-(3-phenoxy-phenyl)-ureido]-benzoic acid

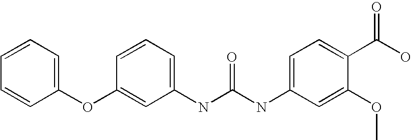

Using the same procedure as described in Ex 9 was the title product synthesised from 4-amino-2-methoxy benzoic acid and 3-Phenoxyphenylisocyanate.

¹H-NMR (DMSO-d₆): δ 12.07 (br s, 1H), 9.02 (s, 1H), 8.93 (s, 1H), 7.66 (d, 1H), 7.40 (m, 3H), 7.29 (m, 2H), 7.15 (m, 2H), 7.03 (m, 2H), 6.95 (dd, 1H), 6.64 (dd, 1H), 3.79 (s, 3H)

LC-MS(an20n15): tr=8.8 min. (M−1)=377.0 m/z

### Example 154

### N-[3-(Benzyl-Isopropyl-amino)-propyl]-2-methoxy-4-[3-(3-phenoxy-phenyl)-ureido]-benzamide

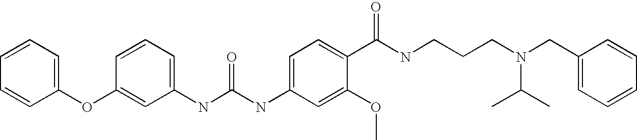

Using the same procedure as described in Ex 10 was the title product synthesised from Ex 138 and N*1*-Benzyl-N*1*-isopropyl-propane-1,3-diamine (synthesized as described in example 77 using Bromopropyl phtalimide and Benzyl-isopropyl-amine)

¹H-NMR (CDCl₃): δ 9.88 (s, 1H), 9.66 (s, 1H), 8.63 (s, 1H), 8.15 (t, 1H), 7.81 (d, 1H), 7.50 (d, 1H), 7.45 (t, 1H), 7.39 (m, 2H), 7.29 (m, 6H), 7.05 (m, 2H), 6.97 (m, 2H), 6.61 (m, 1H), 3.94 (s, 2H), 3.80 (s, 3H), 3.37 (m, 3H), 2.87 (t, 2H), 1.91 (t, 2H), 1.26 (d, 6H)

LC-MS(an20p15): tr=6.3 min. (M+1)=567.1 m/z

### Example 155

### (2-{2-Methoxy-4-[3-(4-trifluoromethoxy-phenyl)-ureido]-benzoylamino}-ethyl)-methyl-carbamic acid tert-butyl ester

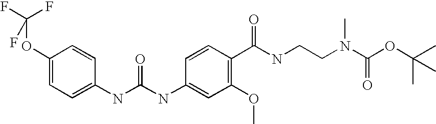

Using the same procedure as described in Ex 48 was the title product synthesised from Ex 138 and commercially available (2-Amino-ethyl)-methyl-carbamic acid tert-butyl ester NMR(CDCl3): δ 1.41 (s, 9H), 2.90 (s, 3H), 3.96 (s, 3H), 8.55 (s, 1H), 8.61 (1H)

LC-MS(an20p10): Rt=8.81 min. (M+1)=527.1 m/z

### Example 156

### 2-Methoxy-N-(2-methylamino-ethyl)-4-[3-(4-trifluoromethoxy-phenyl)-ureido]-benzamide

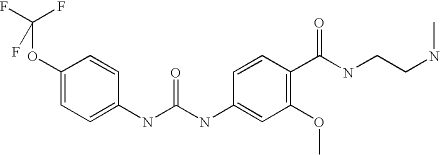

Ex 155 (0.7 g, 1.33 mmol) was suspended in EtOAc and cooled to 0° C. before anhydrous hydrogenchloride was bubbled through the solution for 10 min. Stirring was continued for 1 h before the volatiles were removed in vacuo. The residue was partitioned between Sat, NaHCO₃-solution (100 ml) and DCM (100 ml). The aqueous phase was extracted with DCM (3×70 ml). The combined organic extracts were dried over MgSO₄. Solvent was removed in vacuo to give the title compound Ex 156 (0.52 g, 1.22 mmol, 92%).

NMR(CDCl3): δ 2.46 (s, 3H), 3.92 (s, 3H), 8.49 (t, 1H), 8.82(s, 1H), 8.83 (s, 1H)

### Example 157

### N-[2-(Benzo[1,3]dioxol-5-ylmethyl-methyl-amino)-ethyl]-2-methoxy-4-[3-(4-trifluoromethoxy-phenyl)-ureido]-benzamide

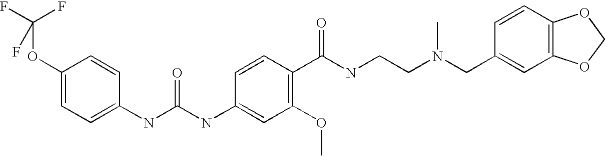

To a solution of piperonylalcohol (36 mg, 0.235 mmol) in THF (30 ml) were successively added triphenylphosphine (92 mg, 0.352 mmol), Ex 156 (100 mg, 0.235 mmol) and DIAD (68 μl, 0.352 mmol). The mixture was stirred overnight at room temperature under N₂. The reaction mixture was partitioned between sat. NaHCO₃-solution (100 ml) and EtOAc (100 ml). The aqueous phase was extracted with EtOAc (3×70 ml). The combined organic extracts were dried over MgSO₄ and concentrated in vacuo. The crude was purified over silica gel chromatography (eluted with DCM/MeOH/NH₃ (100:10:1)) to give the title compound Ex 157 (8.8 mg, 0.016 mmol, 6.8%)

NMR(CDCl₃): δ 2.23 (s,3H), 3.44 (s, 2H), 3.95 (s, 3H), 5.89 (s, 2H), 8.78 (s, 1H), 8.90 (s, 1H)

LCMS(an20p10): Rt=6.10 min. (M+1)=561.1 m/z

### Example 158

### Isobutyric acid 3-isobutyrylamino-benzyl ester

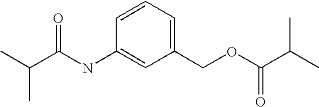

A solution of 3-aminobenzyl alcohol (1 g, 8.32 mmol), isobutyric anhydride (2.69 ml, 16.4 mmol) and DMAP (0.05 g, 0.4 mmol) in dry dichloromethane (30 ml) was stirred overnight at room temperature. Solvent was removed in vacuo to give a residue which was partitionned between ethyl acetate and 1N aq. HCl solution. The organic phase was washed with 1N aq. HCl (2×), sat.aq. NaHCO3, brine and dried over MgSO4 to give the title compound Ex 158 as a pale brown oil (2.19 g, 8.32 mmol, 100%).

NMR(CDCl3): δ 1.20 (d, 6H), 1.25 (d, 6H), 2.55 (m, 2H), 5.09 (s, 2H), 7.09 (d, 1H), 7.28-7.56 (m, 4H)

### Example 159

### N-(3-Bromomethyl-phenyl)-isobutyramide

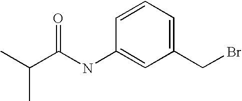

To a solution of Ex 158 (2.19 g, 8.32 mmol) in dry dichloromethane (25 ml) was added a 30% solution of HBr in acetic acid (85 ml, excess). The reaction mixture was stirred for 3 days at room temperature. The mixture was then poured onto ice (400 g) and extracted with dichloromethane. The organic phase was washed with water (1×), sat. aq. NaHCO3, brine, dried over MgSO4 and concentrated in vacuo to give the title compound Ex 159 as a white solid (1.9 g, 7.4 mmol, 84%).

NMR(CDCl3): δ 1.25 (d, 6H), 2.51 (m, 1H), 4.44 (s, 1H), 7.11 (d, 1H), 7.25-7.43 (m, 3H), 7.66 (s, 1H).

### Example 160

### N-{2-[(3-Isobutyrylamino-benzyl)-methyl-amino]-ethyl}-2-methoxy-4-[3-(4-trifluoromethoxy-phenyl)-ureido]-benzamide

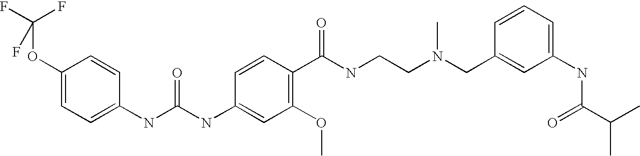

To a solution of Ex 156 (50 mg, 0.117 mmol) and potassium carbonate (excess) in DMF (7 ml) was added Ex 159 (30 mg, 0.117 mmol). The mixture was refluxed at 70° C. overnight under N₂. The reaction mixture was partitioned between a 2 M NaHSO₃-solution (20 ml) and EtOAc (20 ml). The aqueous phase was extracted with EtOAc (2×20 ml). The combined organic extracts were dried over MgSO₄ and concentrated in vacuo. The crude was purified over silica gel chromatography (eluted with DCM/MeOH/NH₃ (100:10:1)) to give the title compound Ex 160 (17.1 mg, 0.028 mmol, 24%)

NMR(CDCl3): δ 1.20 (d, 6H), 2.23 (s, 3H), 3.86 (s, 3H), 8.53 (t, 1H), 8.67(s, 1H), 8.76 (s, 1H)

LC-MS(an20p10): Rt=6.16 min. (M+1)=602.2 m/z

### Example 161

### 4-Ethylamino-2-methoxy-benzoic acid methyl ester

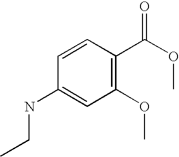

Using the same procedure as described in Ex 30 was the title product synthesised from acetyldehyde and methyl 4-amino-2-methoxybenzoate

¹H-NMR (CDCl₃): δ 7.73 (d, 1H), 6.13 (dd, 1H), 6.06 (d, 1H), 4.25 (br s, 1H), 3.83 (s, 3H), 3.80 (s,3H), 3.18 (q, 2H), 1.24 (t, 3H)

### Example 162

### 4-[1-Ethyl-3-(4-trifluoromethoxy-phenyl)-ureido]-2-methoxy-benzoic acid methyl ester

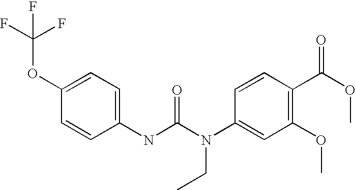

Using the same procedure as described in example 31 was the title product synthesised from Ex 161 and 4-trifluoromethoxyphenylisocyanate

¹H-NMR (CDCl₃): δ 7.82 (d, 1H), 7.35 (m, 2H), 7.06 (d, 2H), 6.87 (m, 2H), 6.54 (s, 1H), 3.86 (s, 3H), 3.84 (s, 3H), 3.77 (q, 2H), 1.15 (t, 3H)

### Example 163

### 4-[1-Ethyl-3-(4-trifluoromethoxy-phenyl)-ureido]-2-methoxy-benzoic acid

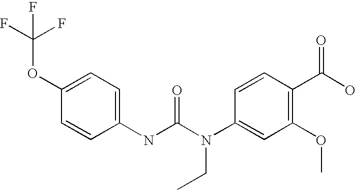

Using the same procedure as described in example 32 was the title product synthesised from Ex 162

¹H-NMR (CDCl₃): δ 10.40 (br s, 1H), 8.18 (d, 1H), 7.42 (m, 2H), 7.13 (m, 3H), 7.00 (d, 1H), 6.58 (s, 1H), 4.10 (s, 3H) 3.86 (q, 2H), 1.23 (t, 3H)

### Example 164

### N-(2-Diethylamino-ethyl)-4-[1-ethyl-3-(4-trifluoromethoxy-phenyl)-ureido]-2-methoxy-benzamide

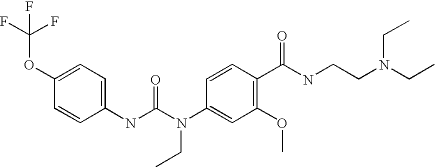

Using the same procedure as described in Ex 33 was the title product synthesised from Ex 163 and N*1*,N*1*-Diethyl-ethane-1,2-diamine

¹H-NMR (CDCl₃): δ 8.48 (br s, 1H), 8.30 (d, 1H), 7.34 (m, 2H), 7.11 (d, 2H), 7.03 (dd, 1H), 6.90 (d, 1H), 6.26 (s, 1H), 4.00 (s, 3H), 3.83 (q, 2H), 3.64 (m, 2H), 2.74 (m, 6H), 1.18 (m, 9H)

LC-MS(an20p15): tr=4.2 min. (M+1)=497.0 m/z

### Example 165

### Ethyl-(4-trifluoromethoxy-phenyl)-amine

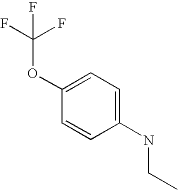

Using the same procedure as described in Ex 30 was the title product synthesised from acetyldehyde and 4-trifluoromethoxyaniline

¹H-NMR (CDCl₃): δ 7.08 (m, 2H), 6.58 (m, 2H), 3.62 (br s, 1H), 3.16 (q, 2H), 1.29 (t, 3H)

### Example 166

### 4-[3-Ethyl-3-(4-trifluoromethoxy-phenyl)-ureido]-2-methoxy-benzoic acid methyl ester

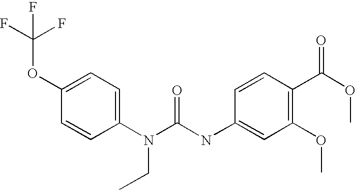

Using the same procedure as described in Ex 169 was the title product synthesised from Ex 165 and methyl 4-amino-2-methoxybenzoate

¹H-NMR (CDCl₃): δ 7.69 (d, 1H), 7.52 (d, 1H), 7.33 (s, 4H), 6.49 (dd, 1H), 6.32 (s, 1H), 3.87 (s, 3H), 3.80 (s, 3H), 3.77 (q, 2H), 1.17 (t, 3H)

### Example 167

### 4-[3-Ethyl-3-(4-trifluoromethoxy-phenyl)-ureido]-2-methoxy-benzoic acid

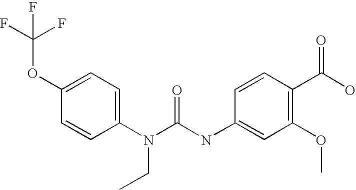

Using the same procedure as described in Ex 32 was the title product synthesised from Ex 166

¹H-NMR (CDCl₃): δ 10.44 (br s, 1H), 7.90 (d, 1H), 7.80 (d, 1H), 7.35 (s, 4H), 6.50 (m, 2H), 4.05 (s, 3H), 3.78 (q, 2H), 1.19 (t, 3H)

### Example 168

### N-(2-Diethylamino-ethyl)-4-[3-ethyl-3-(4-trifluoromethoxy-phenyl)-ureido]-2-methoxy-benzamide

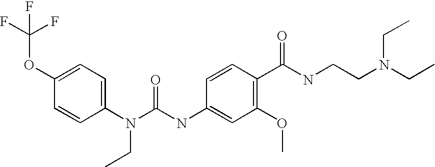

Using the same procedure as described in Ex 33 was the title product synthesised from Ex 167 and N*1*,N*1*-Diethyl-ethane-1,2-diamine

¹H-NMR (CDCl₃): δ 8.43 (br t, 1H), 8.02 (d, 1H), 7.72 (d, 1H), 7.37 (s, 4H), 6.41 (dd, 1H), 6.22 (s, 1H), 4.00 (s, 3H), 3.80 (q, 2H), 3.62 (m, 2H), 2.72 (m, 6H), 1.17 (m, 9H)

### Example 169

### 4-[3-(2-Bromo-4-trifluoromethoxy-phenyl)-ureido]-2-methoxy-benzoic acid methyl ester

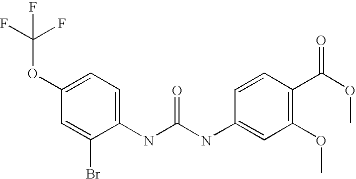

To a cooled (0° C.) solution of phosgene (20% solution in toluene, 2.76 ml, 5.52 mmol) in dry dichloromethane (75 ml) was added, under an argon atmosphere, methyl 4-amino-2-methoxybenzoate (1 g, 5.52 mmol) in one portion, followed by a dropwise addition of diisopropylethylamine (1.92 ml, 11.04 mmol). The mixture was stirred for 15 minutes at 0° C. prior to the addition of 2-bromo-4(trifluoromethoxy)aniline (0.83 ml, 5.52 mmol). The reaction mixture was stirred at 0° C. for a further 2 hours and then was allowed to stir at room temperature overnight. The organic phase was washed with 1N aq. HCl (2×), sat. aq. NaHCO₃, dried over MgSO₄ and concentrated in vacuo to give a solid residue which was recrystallized in hot acetonitrile. The fine crystalline solid was filtered off, washed with cold acetonitrile and dried in vacuo to give the title compound Ex 169 as a pale-orange solid (1.64 g, 3.54 mmol, 64%).

¹H-NMR (DMSO-d₆): δ 3.74 (s, 3H), 3.80 (s, 3H), 7.02 (d, 1H), 7.38 (s, 1H), 7.42 (d, 1H), 7.69 (d, 1H), 7.74 (s, 1H), 8.17 (d, 1H), 8.36 (s, 1H), 9.85 (s, 1H)

### Example 170

### 4-[3-(2-Bromo-4-trifluoromethoxy-phenyl)-ureido]-2-methoxy-benzoic acid, sodium salt

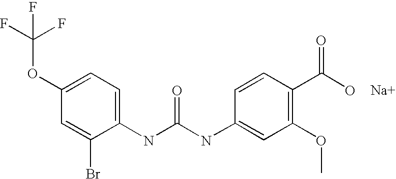

A solution of Ex 169 (1.38 g, 2.98 mmol) and LiOH.H₂O (0.25 g, 5.96 mmol) in a THF/water mixture (40 ml/13 ml) was stirred at 40° C. for 24 hours. THF was removed in vacuo. The aqueous phase was left overnight at room temperature. A white solid crystallized out. The solid was filtered off, washed with several portions of cold water and dried in vacuo to give the title compound Ex 170 as a white solid (1.2 g, 2.54 mmol, 86%).

¹H-NMR (DMSO-do): δ 3.7 (s, 3H), 6.96 (d, 1H), 7.31 (s, 1H), 7.38 (d, 1H), 7.49 (d, 1H), 7.69 (s, 1H), 8.05 (d, 1H), 9.30 (bs, 1H),10.57 (bs, 1H)

### Example 171

### Methyl-(4-phenoxy-phenyl)-amine

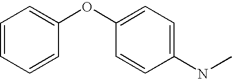

Using the same procedure as described in Ex 30 was the title product synthesised from 4-phenoxyaniline

¹H-NMR (CDCl₃): δ 2.86 (s, 3H), 6.63 (d, 2H), 6.93-7.32 (m, 8H)

### Example 172

### 2-Methoxy-4-[3-methyl-3-(4-phenoxy-phenyl)-ureido]-benzoic acid methyl ester

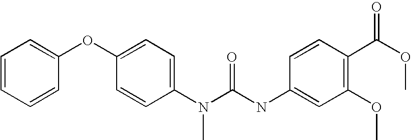

Using the same procedure as described in Ex 169 was the title product synthesised from Ex 171 and methyl 4-amino-2-methoxybenzoate

¹H-NMR (CDCl₃): δ 3.32 (s, 3H), 3.84 (s, 3H), 3.91 (s, 3H), 6.44 (m, 2H), 7.07-7.73 (m, 11H)

### Example 173

### 2-Methoxy-4-[3-methyl-3-(4-phenoxy-phenyl)-ureido]-benzoic acid

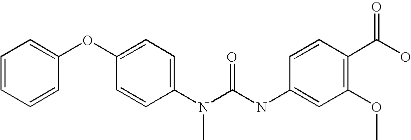

Using the same procedure as described in Ex 32 was the title product synthesised from Ex 172

¹H-NMR (CDCl₃): δ 3.35 (s, 3H), 4.11 (s, 3H), 6.42 (dd, 1H), 6.54 (s, 1H), 7.09-7.46 (m, 9H), 7.98 (m, 2H)

### Example 174

### N-(2-Diethylamino-ethyl)-2-methoxy-4-[3-methyl-3-(4-phenoxy-phenyl)-ureido]-benzamide

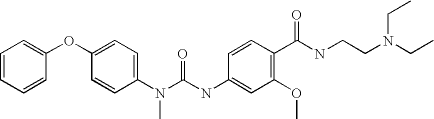

Using the same procedure as described in Ex 33 was the title product synthesised from Ex 173 and N*1*,N*1*-Diethyl-ethane-1,2-diamine

NMR(CDCl3): δ 1.11 (s, 6H), 2.67 (m, 6H), 3.35 (s, 3H), 3.40 (s, 3H), 8.06 (d, 1H), 8.10 (d, 1H)

### Example 175

### 2-Methoxy-4-[3-methyl-3-(4-phenoxy-phenyl)-ureido]-N-(3-piperidin-1-yl-propyl)-benzamide

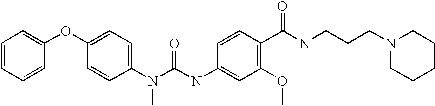

Using the same procedure as described in Ex 33 was the title product synthesised from Ex 173 and 3-Piperidin-1-yl-propylamine

NMR(CDCl3): δ 2.45 (m, 6H), 3.34 (s,3H), 3.40 (s, 3H), 7.94 (t, 1H), 8.00 (d, 1H)

### Example 176

### (2-Benzyloxy-ethyl)-(4-phenoxy-phenyl)-amine

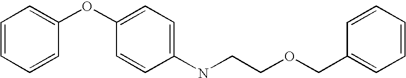

A mixture of 4-phenoxyaniline (1 g, 5.40 mmol) and benzaloxyacetaldehyde (0.76 ml, 5.40 mmol) in methanol (6 ml) was stirred at 0° C. for 30 minutes prior to the dropwise addition of sodium cyanoborohydride (0.339 g, 5.40 mmol). The reaction mixture ws stirred overnight at room temperature. Solvent was removed in vacuo. The residue was partitioned between dichloromethane and brine. The aqueous phase was extracted with dichloromethane. The combined organic phases was dried over MgSO₄ and concentrated in vacuo. The crude was purified over silicagel chromatography (EtOAC/Heptane: 1/9 to 1/1 in 20 min.) to give the title compound Ex 176 (0.74 g, 2.32 mmol, 43%).

NMR(CDCl₃): δ 3.34 (t, 2H), 3.74 (t, 3H), 4.59 (s, 2H), 6.66 (d, 2H), 6.91-7.38 (m, 13H)

### Example 177

### 4-[3-(2-Benzyloxy-ethyl)-3-(4-phenoxy-phenyl)-ureido]-2-methoxy-benzoic acid methyl ester

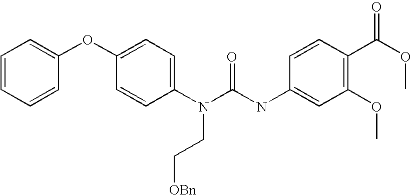

Using the same procedure as described in Ex 169 was the title product synthesised from Ex 176 and methyl 4-amino-2-methoxybenzoate

NMR(CDCl3): δ 3.74 (t, 2H), 4.11 (s, 3H), 4.13 (s, 3H), 3.97 (t, 2H), 4.58 (s, 2H), 6.37 (d, 1H), 7.03-7.43 (m, 15H), 7.54 (s, 1H), 7.7 (d, 1H)

### Example 178

### 4-[3-(2-Hydroxy-ethyl)-3-(4-phenoxy-phenyl)-ureido]-2-methoxy-benzoic acid methyl ester

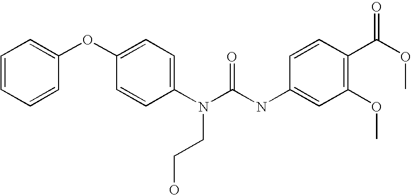

To a solution of Ex 177 (830 mg, 1.57 mmol) in methanol (80 ml) was added 10% Pd(OH)₂/C (10% w/w, 83 mg). The reaction mixture was stirred for 5 hours at 30° C. under a hydrogen atmosphere. The catalyst was filtered off and the filtrate was concentrated in vacuo to give the title compound Ex 178 as a colourless oil (643 mg, 1.47 mmol, 93%)

¹H-NMR (CDCl₃): δ 3.84 (m+s, 5H), 3.9 (m+s, 5H), 6.45 (m, 1H), 7.09-7.45 (m, 9H), 7.51 (s, 1H), 7.74 (d, 1H)

### Example 179

### 2-Methoxy-4-[2-oxo-3-(4-phenoxy-phenyl)-imidazolidin-1-yl]-benzoic acid methyl ester

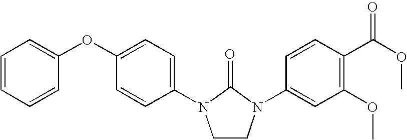

To a cooled (0° C.) solution of Ex 178 (640 mg, 1.47 mmol) in dry dichloromethane (15 ml) were successively added, under an argon atmosphere, methanesulfonyl chloride (0.11 ml, 1.47 mmol) and diisopropylethylamine (0.26 ml, 1.47 mmol). The reaction mixture was stirred for 2 hours at 0° C. and then was allowed to stir at room temperature overnight. Solvent was removed in vacuo. The crude was chromatographed over silica gel to give 4-[3-(2-Methanesulfonyloxy-ethyl)-3-(4-phenoxy-phenyl)-ureido]-2-methoxy-benzoic acid methyl ester (200 mg, 0.39 mmol) which was dissolved in dry acetonitrile (10 ml). Triethylamine (0.54 ml, 3.90 mmol) was added and the reaction mixture was stirred at 70° C. for 2 hours. Solvent was removed in vacuo. The residue was purified over silica gel chromatography (EtOAc/Heptane: 1/9 to 4/1 in 30 min.) to give the title compound Ex 179 (100 mg, 0.24 mmol, 16%).

¹H-NMR (CDCl₃): δ 3.87 (s, 3H), 3.9 (s, 3H), 4.09 (t, 2H), 4.53 (t, 2H), 6.69 (s, 1H), 6.75 (d, 1H), 7.01-7.72 (m, 9H), 7.81 (d, 1H).

LC-MS(an20p15): Rt=7.34 min. (M+1)=419 m/z

### Example 180

### 2-Methoxy-4-[2-oxo-3-(4-phenoxy-phenyl)-imidazolidin-1-yl]-benzoic acid

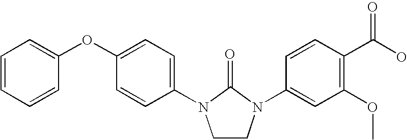

Using the same procedure as described in Ex 32 was the title product synthesised from Ex 179

¹H-NMR (CDCl₃): δ 3.72 (t, 2H), 4.06 (t, 2H), 4.11 (s, 3H), 6.42 (d, 1H), 6.48 (s, 1H), 7.1-7.47 (m, 8H)-7.92 (s, 1H), 8.0 (d, 1H)

### Example 181

### N-(2-Diethylamino-ethyl)-2-methoxy-4-[2-oxo-3-(4-phenoxy-phenyl)-imidazolidin-1-yl]-benzamide

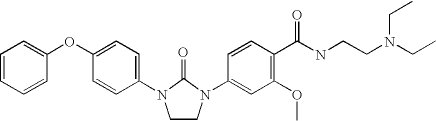

Using the same procedure as described in Ex 33 was the title product synthesised from Ex 180 and N*1*,N*1*-Diethyl-ethane-1,2-diamine

¹H-NMR (CDCl₃): δ 1.28 (br t, 6H), 3.0 (m, 6H), 3.8 (m, 2H), 3.97 (s, 3H), 4.05 (t, 2H), 4.5 (t, 2H), 6.68 (s, 1H), 6.8 (d, 1H), 6.95-7.35 (m, 7H), 7.68 (d, 2H), 8.05 (d, 1H), 8.5 (brs, 1H)

LC-MS(an20p10): Rt=4.75 min. (M+1)=503 m/z

### Example 182

### 4-Amino-5-fluoro-2-methoxy-benzonitrile

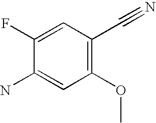

To a cooled (0° C.) solution of methanol (5.2 ml, 130.0 mmol) in anhydrous THF (30 ml) was added, under an argon atmosphere, a 1M solution of tert BuOK in THF (25.9 ml, 25.9 mmol). After stirring for 5 minutes at room temperature, 4-amino-2,5-difluoro-benzonitrile (2 g, 13.0 mmol) was added to the solution in one portion. The reaction mixture was then heated to 70° C. and stirred for 2 h 30 minutes. After cooling, diethyl ether was added. The organic phase was washed with sat. aq. NaHCO₃, brine, dried over MgSO₄ and concentrated in vacuo. The crude was chromatographed over silica gel (EtOAc/Heptane: 1/9 to 1/1) to give the title compound Ex 182 as a pale-yellow solid (1.58 g, 9.51 mmol, 73%).

¹H-NMR (CDCl₃): δ 3.84 (s, 3H), 4.26 (brs, 2H), 6.27 (d, 1H), 7.12 (d, 1H)

### Example 183

### 4-Amino-5-fluoro-2-methoxy-benzoic acid methyl ester

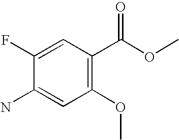

To a saturated solution of gas hydrogen chloride in methanol (20 ml) and water (0.04 ml) was added Ex 182 (290 mg, 1.74 mmol). The reaction mixture was stirred overnight at 40° C. and then at 70° C. for 5 hours. Solvent was removed in vacuo. The residue was partitioned between sat.aq. NaHCO₃ and dichloromethane. The aqueous phase was extracted with dichloromethane (2×). The organic phases were combined, washed with brine, dried over MgSO₄ and concentrated in vacuo The crude was purified over silica gel chromatography (EtOAc/Heptane: 1/9 to 1/1) to give the title compound Ex 183 as a white solid (60 mg, 0.30 mmol, 17%).

¹H-NMR (CDCl₃): δ 3.83 (s, 3H), 3.84 (s, 3H), 6.3 (d, 1H), 7.56 (d, 1H)

### Example 184

### 5-Fluoro-2-methoxy-4-[3-(4-phenoxy-phenyl)-ureido]-benzoic acid methyl ester

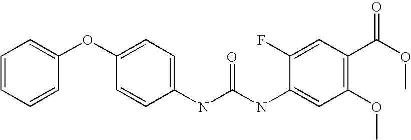

Using the same procedure as described in example 190 was the title product synthesised from Ex 183 and 4-phenoxyaniline

¹H-NMR (DMSO-d₆): δ 3.76 (s, 3H), 3.79 (s, 3H), 6.95-7.57 (m, 10H), 8.15 (d, 1H), 8.85 (s, 1H), 9.15 (s, 1H)

### Example 185

### 5-Fluoro-2-methoxy-4-[3-(4-phenoxy-phenyl)-ureido]-benzoic acid

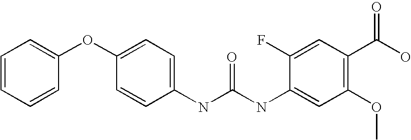

Using the same procedure as described in example 32 was the title product synthesised from Ex 184. Ex 185 was used without characterization in Ex 186.

### Example 186

### 5-Fluoro-2-methoxy-4-[3-(4-phenoxy-phenyl)-ureido]-N-(3-piperidin-1-yl-propyl)-benzamide

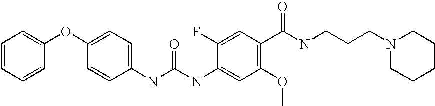

Using the same procedure as described in Ex 33 was the title product synthesised from Ex 185 and 3-Piperidin-1-yl-propylamine

¹H-NMR (CDCl₃): δ 1.4-1.9 (m, 8H), 2.4 (m, 6H), 3.5 (m, 2H), 3.95 (s, 3H), 6.98-7.45 (m, 8H), 7.86 (d, 1H), 8.28 (m, 2H), 8.54 (s, 1H), 9.02 (s, 1H)

LC-MS(an20p15): Rt=6.71 (M+1)=521 m/z

### Example 187

### In Vitro Tests of Compounds According to the Invention

The following results were obtained

Receptor Binding Data

| | | Receptor | |
|---|---|---|---|
| | | binding | IP3 |
| Compound | Example | IC₅₀ μM | IC₅₀ μM |
| 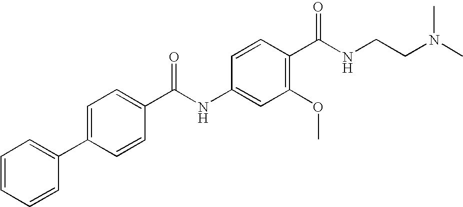 | Ex 2 | 1.48 | |
| 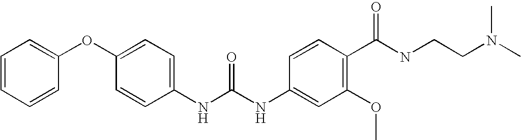 | Ex 8 | 0.38 | 2.3 |
| 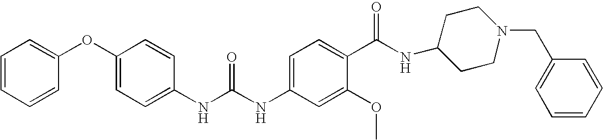 | Ex 16 | 0.22 | 1.8 |
| 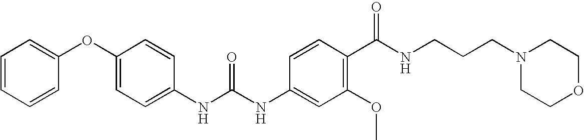 | Ex 23 | 0.21 | 0.77 |
| 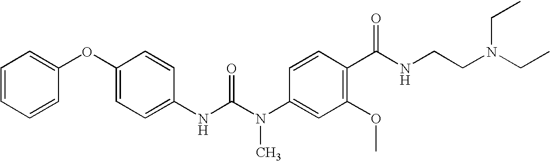 | Ex 33 | 0.048 | 0.29 |
| 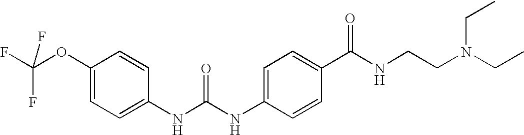 | Ex 47 | 0.07 | 0.29 |
| 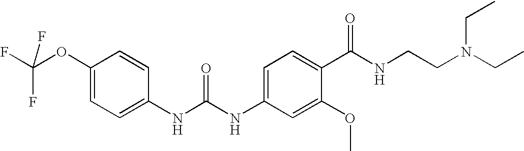 | Ex 48 | 0.096 | 0.19 |
| 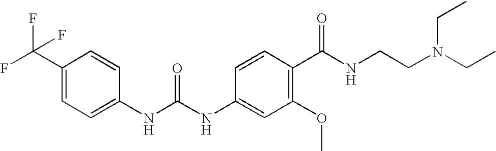 | Ex 67 | 0.027 (SPA) | 0.22 |
| 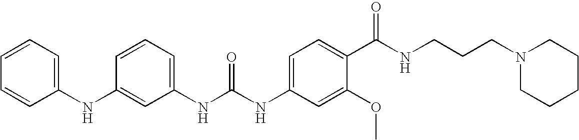 | Ex 89 | 0.012 | 0.022 |
| 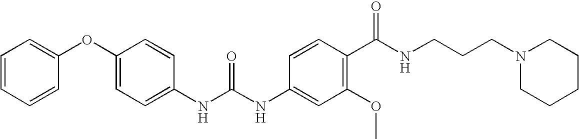 | Ex 95 | 0.044 | 0.24 |
| 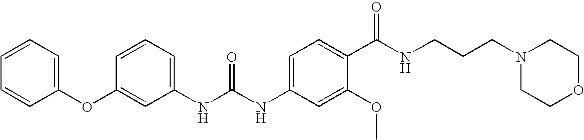 | Ex 121 | 0.074 | 0.11 |
| 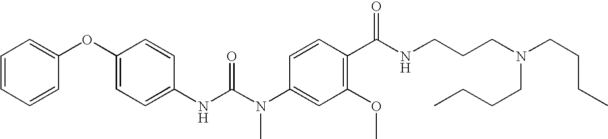 | Ex 134 | 0.069 | 0.67 |
| 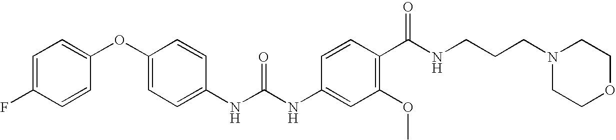 | Ex 135 | 0.45 | 1.6 |
| 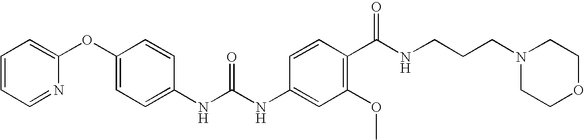 | Ex 136 | 4.45 | |
| 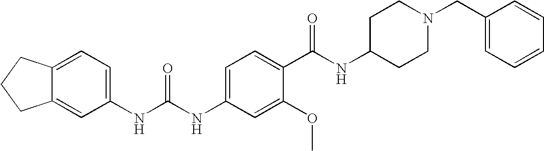 | Ex 137 | 0.30 | |
| 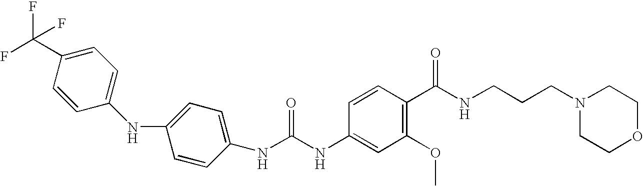 | Ex 139 | 1.41 | |
| 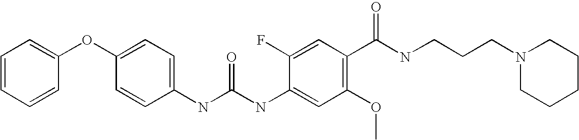 | Ex 186 | 1 | |

### Example 188

In Vivo Tests of Compounds According to the Invention

The following results were obtained on reduction in food intake.

[image]

The following compounds are prepared as described in previous examples.

The following compounds are prepared as described in previous examples.
4-[3-(3-Chloro-phenyl)-ureido]-N-(2-dimethylamino-ethyl)-2-methoxy-benzamide
N-(2-Dimethylamino-ethyl)-2-methoxy-4-(3-phenyl-ureido)-benzamide
N-(2-Diethylamino-ethyl)-2-methoxy-4-(3-phenyl-ureido)-benzamide
N-{3-[4-(4-Acetylamino-phenyl)-piperidin-1-yl]-propyl}-2-methoxy-4-(3-phenyl-ureido)-benzamide
N-{3-[4-(4-Acetylamino-phenyl)-piperidin-1-yl]-propyl}-4-[3-(4-chloro-phenyl)-ureido]-2-methoxy-benzamide
N-{3-[4-(4-Acetylamino-phenyl)-piperidin-1-yl]-propyl}-2-methoxy-4-[3-(4-methoxy-phenyl)-ureido]-benzamide
N-{3-[4-(3-Acetylamino-phenyl)-piperidin-1-yl]-propyl}-2-methoxy-4-(3-phenyl-ureido)-benzamide
2-Methoxy-N-(3-morpholin-4-yl-propyl)-4-(3-phenyl-ureido)-benzamide
2-Methoxy-N-(3-morpholin-4-yl-propyl)-4-(3-phenyl-1-methyl-ureido)-benzamide
4-[3-(4-Chloro-phenyl)-ureido]-2-methoxy-N-(3-morpholin-4-yl-propyl)-benzamide
4-[3-(4-Chloro-phenyl)-1-methyl-ureido]-2-methoxy-N-(3-morpholin-4-yl-propyl)-benzamide
2-Methoxy-4-[3-(4-methoxy-phenyl)-ureido]-N-(3-morpholin-4-yl-propyl)-benzamide
2-Methoxy-4-[3-(4-methoxy-phenyl)-1-methyl-ureido]-N-(3-morpholin-yl-propyl)-benzamide
4-[3-(3-Chloro-phenyl)-ureido]-2-methoxy-N-(3-morpholin-4-yl-propyl)-benzamide
4-[3-(3-Chloro-phenyl)-1-methyl-ureido]-2-methoxy-N-(3-morpholin-4-yl-propyl)-benzamide
4-[3-(3-Iodo-phenyl)-ureido]-2-methoxy-N-(3-morpholinyl-propyl)-benzamide
4-[3-(3-Iodo-phenyl)-1-methyl-ureido]-2-methoxy-N-(3-morpholin-4-yl-propyl)-benzamide
N-(1-Benzyl-piperidin-4-yl)-4-[3-(4-chloro-phenyl)-ureido]-2-methoxy-benzamide
N-(1-Benzyl-piperidinyl)-4-[3-(4-chloro-phenyl)-1-methyl-ureido]-2-methoxy-benzamide
N-(1-Benzyl-piperidin-4-yl)-2-methoxy-4-[3-(4-methoxy-phenyl)-1-methyl-ureido]-benzamide
N-(1-Benzyl-piperidin-4-yl)-2-methoxy-4-[3-(4-methoxy-phenyl)-ureido]-benzamide
N-(1-Benzyl-piperidin-4-yl)-4-[3-(3-chloro-phenyl)-ureido]-2-methoxy-benzamide
N-(1-Benzyl-piperidin-4-yl)-4-[3-(3-chloro-phenyl)-1-methyl-ureido]-2-methoxy-benzamide
N-(1-Benzyl-piperidin-4-yl)-4-[3-(3-Iodo-phenyl)-ureido]-2-methoxy-benzamide
N-(1-Benzyl-piperidin-4-yl)-4-[3-(3-iodo-phenyl)-1-methyl-ureido]-2-methoxy-benzamide
N-(1-Benzyl-piperidin-4-yl)-4-(3-phenyl-ureido)-2-methoxy-benzamide
N-(1-Benzyl-piperidin-4-yl)-4-(3-phenyl-1-methyl-ureido)-2-methoxy-benzamide
N-[2-(4-Benzyl-piperazin-1-yl)-ethyl]-4-[3-(3-iodo-phenyl)-1-methyl-ureido]-2-methoxy-benzamide
N-[2-(4-Benzyl-piperazin-1-yl)-ethyl]-4-[3-(3-iodo-phenyl)-ureido]-2-methoxy-benzamide
N-[2-(4-Benzyl-piperazin-1-yl)-ethyl]-4-[3-(3-chloro-phenyl)-ureido]-2-methoxy-benzamide
N-[2-(4-Benzyl-piperazin-1-yl)-ethyl]-4-[3-(3-chloro-phenyl)-1-methyl-ureido]-2-methoxy-benzamide
N-[2-(4-Benzyl-piperazin-1-yl)-ethyl]-2-methoxy-4-[3-(4-methoxy-phenyl)-1-methyl-ureido]-benzamide
N-[2-(4-Benzyl-piperazin-1-yl)-ethyl]-2-methoxy-4-[3-(4-methoxy-phenyl)-ureido]-benzamide
N-[2-(4-Benzyl-piperazin-1-yl)-ethyl]-4-[3-(4-chloro-phenyl)-ureido]-2-methoxy-benzamide
N-[2-(4-Benzyl-piperazin-1-yl)-ethyl]-4-[3-(4-chloro-phenyl)-1-methyl-ureido]-2-methoxy-benzamide
N-[2-(4-Benzyl-piperazin-1-yl)-ethyl]-2-methoxy-4-(1-methyl-3-phenyl-ureido)-benzamide
N-[2-(4-Benzyl-piperazin-1-yl)-ethyl]-2-methoxy-4-(3-phenyl-ureido)-benzamide

1. A compound with the following structure Formula I) 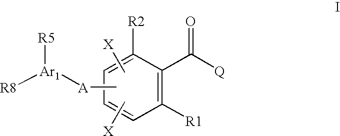
wherein -A- is a linker, which is selected from the group consisting of 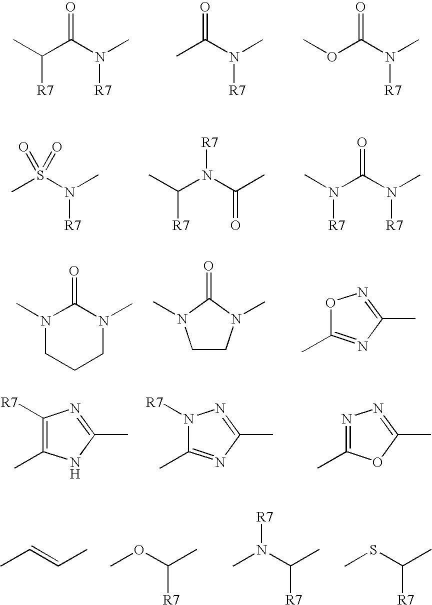
and, wherein the linker -A- may be attached via either of the two free bonds to the Ar₁ group;
and R7 is the same or different and is hydrogen or a straight or branched C₁-C₄ alkyl or alkenyl group;
Ar₁ is an aryl or heteroaryl group;
R1 is a lower alkoxy group alkyl-O— with one to four carbon atoms,
R2 is an R1 group or hydrogen, an OH or an NH₂ group,
Q is selected from the group consisting of 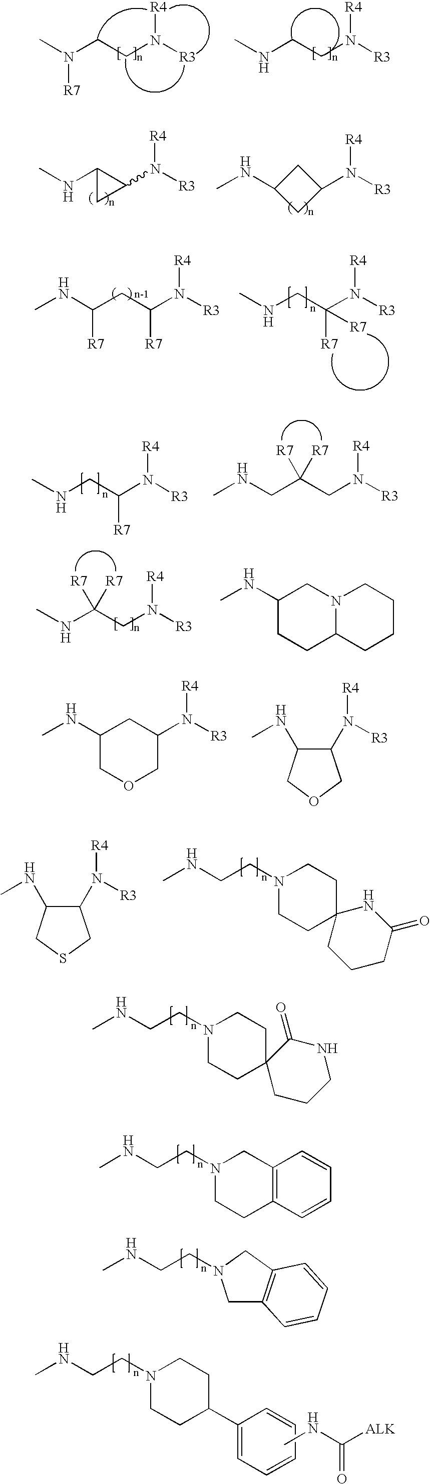 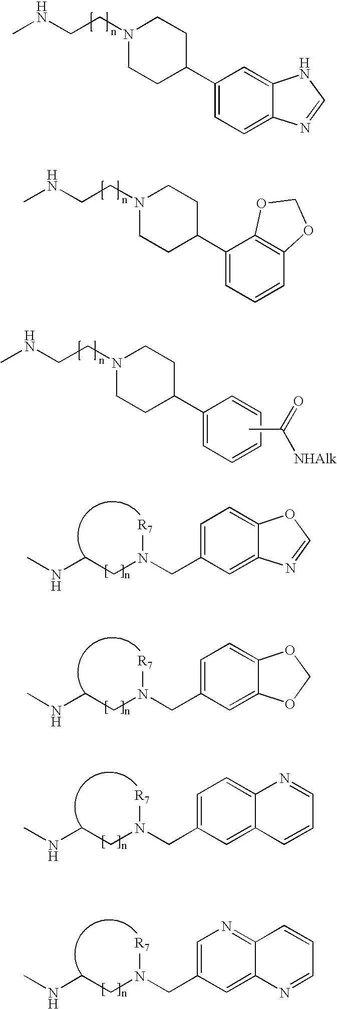
R3 and R4 are the same or different selected from straight or branched alkyl, alkenyl or alkynyl groups with 1-8 carbon atoms; cycloalkyl groups with 3-7 carbon atoms; alkylcycloalkyl with 4-9 carbon atoms; alkylaryl groups alkylheterocyclyl groups the aryl, heterocyclyl and heteroaryl groups may be substituted;
Alk is the same or a different alkyl, alkenyl or alkynyl group;
R3 and R4 may optionally be linked to each other, when possible, as indicated in Formula I; and oxygen or nitrogen atoms may be inserted in the chain or ring in a chemically stable position;
R5 is selected from hydrogen, halogen, alkoxy (AlkO—), hydroxy, alkylamino (AlkNH—), dialkylamino (Alk₂N—), hydroxylalkyl, carboxamido (—CONH₂, —CONHAlk, —CONAlk₂), acylamido (—NHCO-Alk), acyl (—CO-Alk), —CHO, nitrile, alkyl, alkenyl, alkynyl, —SCH₃, partially or fully fluorinated alkyl, alkoxy or thioalkoxy groups;
more than one R5 group, same or different, may be present on Ar₁; when more than one 5 R5 or when one R5 and one R8 group are present they could be connected to each other, directly or with a suitable connecting moiety, to form rings;
each X being the same or different H, F, CI, Br, I, —SCH₃, —CF₃, —OCF₃, —SCF₃, OCH₃, or lower alkyl or alkenyl group;
n is 1, 2 or 3,
R8 is halogen, alkyl, alkenyl, or alkynyl, cycloalkyl with 3-7 carbons, aryl, hetetoaryl, heterocyclyl, alkylcycloalkyl, alkylaryl, alkylheterocyclyl, alkylheteroaryl, arylalkoxy, aryloxy groups, alkoxy, dialkylamino, —CONHAlk, —CONHAr —CONAlk₂, —NHCO-Alk —NHCO—Ar, —CO-Alk, —CO—Ar, —SCH₃, partially or fully fluorinated alkyl, alkoxy or or R8 has the structure 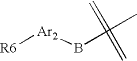
in which B is a single bond or a connecting moiety selected from the group consisting of: 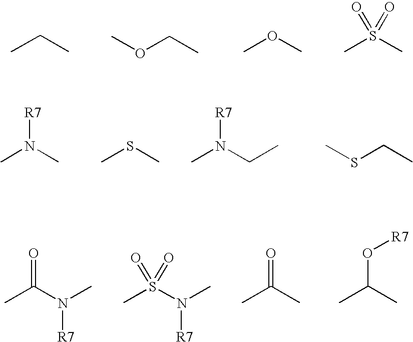
which may be attached via either of the two free bonds to the Arₜ group;
Ar₂ is an aryl or heteroalkyl group;
R6 is selected from hydrogen, halogen, alkoxy (AlkO—), hydroxy, alkylamino (AlkNH—), dialkylamino (Alk₂N—), hydroxylalkyl, carboxamido (—CONH₂, —CONHAlk, —CONAlk₂), acylamido (—NHCO-Alk), acyl (—CO-Alk), —CHO, nitrile, alkyl, alkenyl, alkynyl, —SCH₃, partially or fully fluorinated alkyl, alkoxy or thioalkoxy;
more than one R6 group, same or different, may be present on Ar₂; when more than one R6 group is present they could be connected to each other to form rings.

2. A compound according to claim 1, wherein Q is

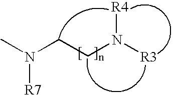

3. A compound cording to claim 1 or 2, wherein R8 is

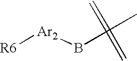

4. A compound according to claim 1, wherein R8 is selected from halogen, alkyl, alkenyl, alkynyl, cycloalkyl groups with 3-7 carbons, aryl (Ar), heteroaryl, heterocyclyl, alkylcycloalkyl, alkylaryl, alkylheterocyclyl, alkylheteroaryl, arylalkoxy, aryloxy (ArO—), alkoxy (AlkO—), dialkylamino (Alk₂N—), —CONHAlk, —CONHAr, —CONAlk₂, —NHCO-Alk, —NHCO—Ar, —CO-Alk —CO—Ar, —CF₃, —OCF₃, —SCF₃, Or SCH₃.

5. A compound according to claim 1 wherein A is selected from the group consisting of: 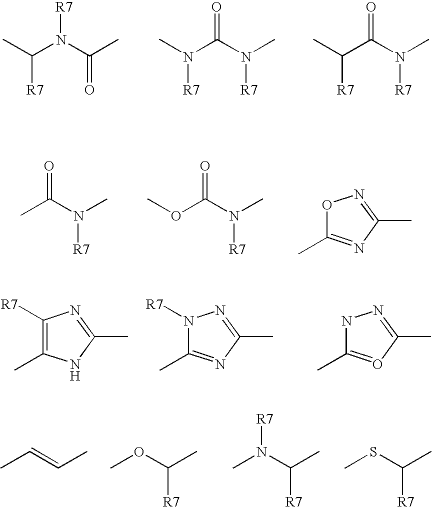
wherein R7 is as defined in claim 1.

6. A compound according to claim 1 wherein A is selected from the group consisting of: 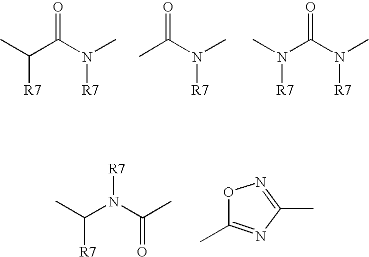
wherein R7 is as defined in claim 1.

7. A compound according to claim 1 wherein B is a single bond or selected from the group consisting of: 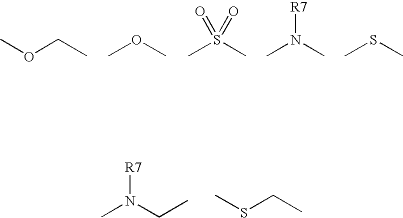
wherein R7 is as defined in claim 1.

8. A compound according to claim 7, wherein B is selected from the group consisting of 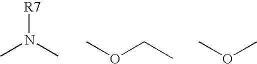
wherein R7 is as defined in claim 1.

9. A compound according to with the following structure 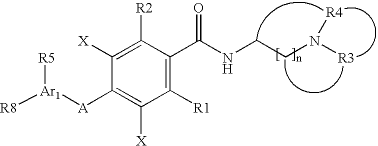
wherein Ar₁, Ar₂, A, B, RI, R2, R3, R4, R5, R6, R7, R8, X and n are defined as in claim 1.

10. A compound according to claim 9, wherein R8 is

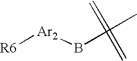

11. A compound according to wherein the —B— moiety is not placed ortho to the -A- linker.

12. A compound according to, wherein Ar₁ and Ar₂ are the same or different aryl or heteroaryl groups.

13. A compound according to wherein R2 is hydrogen.

14. A compound according to claim 1, wherein R2 is hydrogen and X is H, F, Cl, Br, I, CF₃, OCF₃, SCF₃, SCH₃ or lower alkyl or alkenyl group.

15. A compound according to claim 1, wherein R2 is H and X is H or F.

16. A compound according to claim 1, wherein R5 and R6 may be the same or different selected from hydrogen, halogen, alkoxy (AlkO—), alkylamino (AlkNH—), dialkylamino (Alk₂N—), carboxamido (—CONH₂, —CONHAlk, CONAlk₂), acylamido (—NHCO-Alk), nitrile, lower alkyl groups, —CF₃, —OCF₃, —SCF₃, —SCH₃.

17. A compound according to claim 1 in amorphous or crystalline form.

18. A compound according to claim 1 in racemic or enantiomeric 5 form.

19. A compound according to claim 1 in the form of a physiologically acceptable salt, complex, solvate or prodrug thereof.

20-35. (canceled)

36. A cosmetic method for reducing overweight and/or for treating of and/or preventing overweight, bulimia, bulimia nervosa, obesity and/or complications thereto, the method comprising administering to an animal in need thereof, an effective amount of a compound according to claim 1.

37. A method for the treatment and/or prophylaxis of diseases caused by a melanin-concentrating hormone, the method comprising administering to a mammal in need thereof an effective amount of a compound according to claim 1.

38. A method for the treatment and/or prophylaxis of diseases caused by feeding disorders, the method comprising administering to a mammal in need thereof an effective amount of a compound according to claim 1.

39. A method for modifying the feeding behaviour of a mammal, the method comprising administering to a mammal in need thereof an effective amount of a compound according to claim 1.

40. A method for the reduction of body mass, the method comprising administering to a mammal in need thereof an effective amount of a compound according to claim 1.

41. A method for the treatment and/or prophylaxis of Syndrome X (metabolic syndrome) or any combination of obesity, insulin resistance, dyslipidemia, impaired glucose tolerance and hypertension, the method comprising administering to a mammal in need thereof an effective amount of a compound according to claim 1.

42. A method for the treatment and/or prophylaxis of Type II diabetes or Non Insulin Dependent Diabetes Mellitus (NIDDM), the method comprising administering to a mammal in need thereof an effective amount of a compound according to claim 1.

43. A method for the treatment and/or prophylaxis of bulimia, bulimia nervosa and/or obesity, the method comprising administering to a mammal in need thereof an effective amount of a compound according to claim 1.

44. A method for the treatment and/or prophylaxis of depression and/or anxiety, the method comprising administering to a mammal in need thereof an effective amount of a compound according to claim 1.

45. A pharmaceutical composition comprising a compound according to claim 1 or a physiologically acceptable salt thereof together with one or more physiologically acceptable excipients.

46. A pharmaceutical composition according to claim 45, wherein the compound is present in the form of a physiologically acceptable salt an organic acid.

47. A composition according to claim 45 for enteral and/or parenteral use.

48. A pharmaceutical composition according to claim 45 for oral, buccal, rectal, nasal, topical, vaginal or ocular use.

49. A pharmaceutical composition according to claim 45 in the form of a solid, semi-solid or fluid composition.

50. A pharmaceutical composition according to claim 49 in solid form, wherein the composition is in the form of one or more tablets.

51. A pharmaceutical composition according to claim 49 in semi-solid form, wherein the composition is in the form of a chewing gum, an ointment, a cream, a liniment, a paste, a gel or a hydrogel.

52. A pharmaceutical composition according to claim 49 in fluid form, wherein the composition is in the form of a solution, an emulsion, a suspension, a dispersion, a liposomal composition, a spray, a mixture, or a syrup.

53. A pharmaceutical composition according to claim 46 comprising a therapeutically effective amount of a compound according to claims.

54. A pharmaceutical composition according to claim 53, wherein the amount is from about 0.001 mg to about 1 g.

55. (canceled)
